(19) United States
(12) Patent Application Publication
(10) Pub. No.: US 2026/0265715 A1
(43) Pub. Date: Sep. 10, 2026

(54) CHEMICALLY MODIFIED SINGLE STRANDED DNA IMPROVES GENE EDITING EFFICIENCY

(71) Applicant: Research Institute at Nationwide Children's Hospital, Columbus, OH (US)

(72) Inventor: Sriram Vaidyanathan, Columbus, OH (US)

(21) Appl. No.: 19/167,318

(22) PCT Filed: Apr. 11, 2024
(86) PCT No.: PCT/US2024/024134

§ 371 Date: Sep. 19, 2025

Related U.S. Application Data
(60) Provisional application No. 63/459,965, filed on Apr. 17, 2023.

(51) Int. Cl. C12N 9/22; A61K 35/00; A61K 48/00; C12N 15/10; C12N 15/113; C12N 15/52; C12N 15/90

(52) U.S. Cl. CPC C12N 9/222; A61K 35/00; A61K 48/005; C12N 15/102; C12N 15/113; C12N 15/52; C12N 15/90; C12N 2310/20; C12N 2310/33

(57) ABSTRACT

This disclosure provides gene-editing systems and compositions that comprise modified single stranded homology directed repair (HDR) template polynucleotides that improve gene editing. Also provided are methods for using the disclosed gene-editing systems and compositions.

### CROSS-REFERENCE TO RELATED PATENT APPLICATION

This application claims the benefit under 35 U.S.C. § 119 (e) of U.S. Provisional Application No. 63/459,965, filed Apr. 17, 2023, the entire contents of which are incorporated herein by reference.

### STATEMENT AS TO FEDERALLY SPONSORED RESEARCH

This invention was made with government support under Grant No. HL 151900 awarded by the National Institutes of Health. The government has certain rights in the invention.

### BACKGROUND

The present disclosure relates generally to the field of genetic editing. Engineered cell therapies such as chimeric antigen receptor T-cells (CAR-T cells) and gene corrected hematopoietic stem and progenitor cells (HSPCs) have revolutionized the treatment of genetic diseases and cancers. The number of cell therapies is predicted to grow significantly and generate revenues of ~$24 billion dollars by 2030. Currently, marketed products are manufactured using retroviruses which randomly integrate their cargo in the genome. This poses a risk for oncogenic transformation and prevents the treatment of diseases where precise regulation of the gene product is necessary. In addition, current CAR-T cells show heterogenous expression of the cargo resulting in cell exhaustion and sub-optimal efficacy. Non-integrating viruses such as adeno-associated viruses (AAV) cannot be used to engineer rapidly dividing cells such as HSPCs and T-cells since they are diluted out during cell division. Thus, improved methods to engineer HSPCs, T-cells and other tissue resident stem cells are needed to produce more potent and safer cell therapies.

Clustered Regularly Interspaced Short Palindromic Repeat (CRISPR)/Cas (CRISPR/Cas), Zinc Zinger Nuclease (ZNF) and TALEN are examples of gene-editing systems that can induce a double stranded break (DSB) in targeted genomic loci. The DSB is repaired by the error prone non-homologous end joining (NHEJ) or the homology dependent repair (HDR) (also known as homologous recombination (HR)) pathways. HDR can be manipulated to introduce exogenous sequences if a template DNA (HDR template) with homology arms (HA) that resemble the DSB site is delivered to the nucleus in cells. HDR templates packaged in AAV is the current gold standard for high efficiency HDR (30-70% modified stem cells). This method using Cas9 and AAV was used to develop an autologous gene corrected HSPC therapy targeting sickle cell disease. Unfortunately, engraftment of this product prolonged low blood cell counts in the first patient suggesting a problem with engraftment. A competing product made only using Cas9 showed successful engraftment suggesting AAV as the cause for failed engraftment. Indeed, studies have shown that AAV-based HDR templates activate cellular defenses that limit the regenerative potential of the corrected stem cells. Thus, alternative methods to deliver the HDR template are needed to produce safer and more effective stem cell and immunotherapies.

ssDNA can be used as a non-AAV-based HDR template. However, ssDNA templates result in much lower efficiencies in gene editing as compared to AAV-delivered templates. The efficacy of ssDNA is likely reduced by the activity of endogenous exonucleases and endonucleases present within every cell. Therefore, improvements to the efficiency of ssDNA HDR templates are needed to improve precise gene editing technologies.

### SUMMARY OF THE DISCLOSURE

The present disclosure shows, for the first time, that modified internal bases improve the protection of HDR templates (e.g., an ssDNA, an RNA or an RNA/DNA hybrid HDR template) from nuclease-mediated degradation.

An aspect of the disclosure is directed to a gene-editing system comprising, or consisting essentially of, or consisting of: components of a gene editing system selected from a CRISPR-Cas system, a TALEN system and a Zinc Finger Nuclease (ZFN) system; and a single stranded homology directed repair (HDR) template polynucleotide, wherein the first three nucleotides at the 5′ end and the last three nucleotides at the 3′ end of the HDR repair template polynucleotide comprise a chemically modified nucleotide; and wherein the nucleotides between the 5′end and the 3′ end of the HDR repair template polynucleotide comprises a chemically modified nucleotide every 4, 5, 6, 7, 8, 9 or 10 nucleotides along at least 95%, at least 96%, at least 97%, at least 98%, at least 99% or more of the length of the HDR template polynucleotide. In some embodiments, the least 95%, at least 96%, at least 97%, at least 98%, at least 99% or more of the length of the HDR template polynucleotide comprises the entire length of the HDR template polynucleotide, including the first three nucleotides at the 5′ end and the last three nucleotides at the 3′ end of the HDR repair template polynucleotide. In some embodiments, the least 95%, at least 96%, at least 97%, at least 98%, at least 99% or more of the length of the HDR template polynucleotide excludes the first three nucleotides at the 5′ end and the last three nucleotides at the 3′ end of the HDR repair template polynucleotide. In one aspect, one or more of the components of the system are detectably labeled.

One aspect of the disclosure is directed to a gene-editing system comprising, or consisting essentially of, or consisting of: components of a gene editing system selected from a CRISPR-Cas system, a TALEN system and a Zinc Finger Nuclease (ZFN) system; and a single stranded homology directed repair (HDR) template polynucleotide, wherein the HDR repair template polynucleotide comprise a chemically modified nucleotide every 4, 5, 6, 7 or 8 nucleotides along at least 95% (at least 95%, at least 96%, at least 97%, at least 98%, at least 99% or more) of the length of the HDR template polynucleotide. In some embodiments, the at least 95% excludes the three terminal nucleotides at the 5′ and 3′ ends of the HDR template. In some embodiments, the at least 95% (the at least 95%, at least 96%, at least 97%, at least 98%, at least 99% or more) includes the entire length of the HDR template including the three terminal nucleotides at the 5′ and 3′ ends of the HDR template.

One embodiment of the disclosure relates to a gene-editing system comprising, or consisting essentially of, or consisting of: components of a gene editing system selected from a CRISPR-Cas system, a TALEN system and a Zinc Finger Nuclease (ZFN) system; and a single stranded homology directed repair (HDR) template polynucleotide, wherein the first three nucleotides at the 5′ end and the last three nucleotides at the 3′ end of the HDR repair template polynucleotide comprise a chemically modified nucleotide; and wherein the nucleotides between the 5′end and the 3′ end of the HDR repair template polynucleotide comprise a chemically modified nucleotide every 4, 5, 6, 7 or 8 nucleotides along at least 95% of the length of the HDR template polynucleotide. In some embodiments, the at least 95% includes the three modified nucleotides at the 5′ and 3′ ends of the HDR template.

In some embodiments, the chemically modified nucleotide comprises a modification has been shown to make ssDNA resistant to nuclease activity. In some embodiments, the chemically modified nucleotide comprises a phosphorothioate (PS) modification, a 2′-O-methyl modification, methoxypropyl phosphonate (MOP), a constrained ethyl (cEt) modification, a locked nucleic acid (LNA) modification, a 2′ fluoro modification, a 1-Boranotriphosphate modification, methoxypropyl phosphonate (MOP) modification, phosphorodiamidate morpholino oligomer (PMO) modification, or a combination thereof.

In some embodiments, the gene-editing system comprises a CRISPR enzyme or a polynucleotide encoding a CRISPR enzyme, a guide RNA (gRNA) sequence that hybridizes to a target sequence, a tracr mate sequence, a tracr sequence, and optionally wherein the CRISPR enzyme is a type II CRISPR-Cas enzyme, and optionally wherein the type II CRISPR-Cas enzyme is a Cas9 enzyme, and optionally wherein the Cas9 enzyme is an SpCas9 enzyme or an SaCas9 enzyme.

In some embodiments, the CRISPR enzyme is a high-fidelity enzyme. In some embodiments, the high-fidelity enzyme is a Cas9 enzyme having an R691A mutation.

In some embodiments, the gene-editing system comprises a nickase. In some embodiments, the gene-editing system comprises a Cas9 enzyme having one or more mutations at positions corresponding to D10, E762, H840, N854, N863 or D986 of SEQ ID NO: 1. In some embodiments, the Cas9 comprises a mutation selected from D10A, E762A, H840A, N854A, N863A or D986A, or wherein the one or more mutations is in a RuvC1 domain of the Cas9 (corresponding to amino acids 1 to 60 of SEQ ID NO: 1).

In some embodiments, the gene-editing system comprises a TALEN system comprising, or consisting essentially of, or consisting of, from the N-terminus to the C-terminus, a first spacer sequence, a TALE DNA binding domain, a second spacer sequence, and a FokI nuclease catalytic domain fused to the C-terminus, wherein the TALE DNA binding domain comprises TAL repeats, wherein the TALE DNA binding domain is specific for binding to target sequence.

In some embodiments, the gene-editing system comprises a ZFN system comprising, or consisting essentially of, or consisting of at least one zinc finger nuclease, the zinc finger nuclease comprising a zinc finger protein comprising 4, 5 or 6 fingers designated F1 to F4, F1 to F5 or F1 to F6, each finger comprising a recognition helix region that recognizes a target sequence.

Another aspect of the disclosure is directed to a non-naturally occurring or engineered composition comprising, or consisting essentially of, or consisting of: (i) a polynucleotide sequence encoding a CRISPR enzyme, (ii) a CRISPR-Cas system chimeric RNA (chiRNA) polynucleotide sequence comprising: (a) a guide sequence capable of hybridizing to a target sequence, (b) a tracr mate sequence, and (c) a tracr sequence, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence; and (ii) a single stranded homology directed repair (HDR) template polynucleotide that comprises a chemically modified nucleotide every 4, 5, 6, 7 or 8 nucleotides along at least 85% of the template polynucleotide.

Another aspect of the disclosure is directed to a non-naturally occurring or engineered composition comprising, or consisting essentially of, or consisting of: (i) a polynucleotide sequence encoding a CRISPR enzyme, (ii) a CRISPR-Cas system chimeric RNA (chiRNA) polynucleotide sequence comprising: (a) a guide sequence capable of hybridizing to a target sequence, (b) a tracr mate sequence, and (c) a tracr sequence, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence; and (ii) a single stranded homology directed repair (HDR) template polynucleotide that comprises a chemically modified nucleotide every 4, 5, 6, 7, 8, 9 or 10 nucleotides along at least 85% of the template polynucleotide.

Another aspect of the disclosure is directed to a method for modifying an organism comprising, or consisting essentially of, or consisting of delivering the gene-editing system or the composition of the instant disclosure to the organism, optionally wherein the organism is a non-human organism.

Another aspect of the disclosure is directed to a method for modifying the genome of a cell comprising, or consisting essentially of, or consisting of delivering the gene-editing system of or the composition of the instant disclosure to the cell, optionally in vitro or in vivo, or further optionally wherein the cell is a prokaryotic cell or a eukaryotic cell. In some embodiments, the gene-editing system or the composition is delivered via liposomes (e.g., lipid nanoparticles), nanoparticles, exosomes, microvesicles, electroporation or a gene-gun.

A further aspect of the disclosure is an isolated host cell comprising, or consisting essentially of, or consisting of the gene-editing system or the composition as described herein. In one aspect, the host cell is a mammalian or human cell. In a further aspect, the cell is a stem cell. In a further aspect, the cell is an airway stem cell. In a separate aspect, the cell is a human umbilical vein endothelial cell. In a separate aspect, the cell is a Hematopoietic Stem and Progenitor cell.

Another aspect of this disclosure is a pharmaceutical composition comprising, or consisting essentially of, or consisting of a carrier, such as a pharmaceutically acceptable carrier and one or more of: the gene-editing system, the composition or the isolated host cell as described herein. In one aspect, the pharmaceutically acceptable carrier is a liposome (e.g., a lipid nanoparticle), a nanoparticle, an exosome, or a microvesicle.

Also disclosed is a kit comprising, or consisting essentially of, or consisting of one or more of: the gene-editing system, the composition, the isolated host cell, the pharmaceutical composition, as disclosed herein and optional instructions for use.

Another aspect of the disclosure is directed to a method for ex vivo cell therapy comprising, or consisting essentially of, or consisting of administering a subject a cell prepared by the method of the instant disclosure. In some embodiments, the cell is a mammalian cell, optionally a human cell, or optionally a non-human cell. In a further aspect, the cell is a stem cell or progenitor cell, optionally a mammalian stem or mammalian progenitor cell such as a human stem cell or a human progenitor cell.

Another aspect of the disclosure is directed to a method comprising, or consisting essentially of, or consisting of delivering the gene-editing system or the composition of the instant disclosure to a cell, producing clones of the cell, and screening for the clones with altered biological activity (e.g., identifying mutations that may be oncogenic) or assessing the pathogenicity of mutations suspected to be involved in a disease process (e.g., assessing whether a particular mutation is capable of causing cystic fibrosis). The methods also can be used to introduce mutations at target loci in a cell.

### BRIEF DESCRIPTION OF THE DRAWINGS

FIGS. 1A-1C. (A) Cas9 can be used to induce double stranded breaks that can be repaired by HR. HR template DNA with homology arms delivered using AAV is currently used to leverage this process to insert exogenous DNA into cells. (B) Unmodified ssDNA can be used as a HR template but is susceptible to degradation by nucleases. Modification of the ends can protect the template from exonucleases and modification of the internal residues can protect the template from nucleases. (C) Phosphorothioate modification is a possible nucleotide modification.

FIGS. 2A-2G. (A) The schematic of the HR template used in the experiments. The CF Transmembrane Conductance Regulator (“CFTR”, NCBI Entrez Gene: 1080 Ensembl: ENSG00000001626 OMIM®: 602421 UniProtKB/Swiss-Prot: P13569), C-C Motif Chemokine Receptor 5 (“CCR5”, HGNC: 1606 NCBI Entrez Gene: 1234 Ensembl: ENSG00000160791 OMIM®: 601373 UniProtKB/Swiss-Prot: P51681) and Hemoglobin Subunit Beta (“HBB”, HGNC: 4827 NCBI Entrez Gene: 3043 Ensembl: ENSG00000244734 OMIM®: 141900 UniProtKB/Swiss-Prot: P68871) loci were edited in airway stem cells to introduce a 20 bp DNA sequence. (B) In airway cells at the CFTR locus, ssDNA templates with every sixth base modified (41±15%) show significantly higher HR than end-modified ssDNA (24±6%), which are both significantly higher than unmodified ssDNA (15±5%). Modifying every third base results in the least gene insertion (11±15%). Statistical comparison was made using one-way ANOVA. (C) ssDNA modified at every sixth base pair (esDNA) is also more efficient than end-modified ssDNA in the HBB locus. The esDNA templates achieved gene insertion in 51±9% alleles at the 800 nM concentration as opposed to 22±4% for end-modified ssDNA. At 400 nM, esDNA achieved 31±6% gene insertion compared to 7±3% gene insertion achieved by end-modified ssDNA. Statistical comparison was made using two-way ANOVA. (D) Using esDNA improves gene insertion compared to end-modified ssDNA in the CCR5 locus at 200 nM (41±5% vs 17±1%) and 400 nM (55±6% vs 40±4%) but not at 800 nM (53±16% vs 51±10%). Statistical comparison was made using two-way ANOVA. (E) In airway cells, esDNA with OM modification also shows higher HR than unmodified ssDNA (55±19% vs 15±8%) which is not significantly different from HR obtained using AAV templates (82±15%). (n=5 biological replicates for esDNA with PS, OM modifications and ssDNA. n=3 biological replicates for AAV). (F) Chemical modification of every sixth base in ssDNA enables improved gene insertion compared to ssDNA with CTS. (G) Gene insertion of different lengths was attempted in exon 12 of the CFTR locus using esDNA templates to introduce the G551D mutation. Editing was performed with DNA-PKcs inhibition. Template 1 (T1) which replaces 12 bp achieved ~80-90% gene insertion while template 2 (T2) which replaces 34 bp achieved only ~60 bp gene insertion. Statistical comparison was made using one-way ANOVA. For all panels using one-way ANOVA, multiple comparisons was performed using Tukey's test. Sidak's test was used for two-way ANOVA. * **, ***, ** and * represent p<0.0001, p<0.001, p<0.01 and p<0.05 respectively for all panels.

FIGS. 3A-3D. (A) The HBB locus was edited in hematopoietic stem and progenitor cells (HSPCs) using ssDNA modified with phosphorothioate groups either at the end or at every sixth base pair. Improved editing was observed with or without inhibition DNA-PKcs inhibition. (B) Similarly, ssDNA with every sixth nucleotide modified showed improved HR in the CFTR locus in HSPCs. (C) The use of phosphorothioate or 2′-O-methyl modifications in every sixth base pair improved editing in human umbilical vein endothelial cells (HUVECs). (D) Chemical modification was not necessary to obtain highly efficient gene insertion using ssDNA in induced pluripotent stem cells (iPSC).

FIGS. 4A-4B. (A) Exemplary backbone modifications. MOP, methoxypropyl phosphonate; PMO, phosphorodiamidate morpholino oligomer; PO, phosphodiester; PS, phosphorothioate. (B) Exemplary 2′ modifications. cEt, constrained ethyl; F, fluoro; LNA, locked nucleic acid; Me, 2′-O-methyl; MOE, methoxyethyl.

FIGS. 5A-5F. (A) Both esDNA OM and esDNA PS corrected ABCs from CF donors (49±14% and 47±14%, respectively). (n=4 biological replicates). Gene correction of the F508del mutation was confirmed using next-generation sequencing. NGS showed 41.8% alleles with perfect HR and ~5% alleles with imperfect HR. Unintended substitutions were found in only 0.6% of alleles. (B) A representative FACS plot shows that >98% of ABCs were positive for KRT5 and P63 4 days after editing. There was no reduction in KRT5 and P63 expression in the edited ABCs relative to Mock electroporated controls in experiments from three different donors. (C) Edited samples displayed transepithelial resistances (TEER) similar to unedited controls (WT and Mock), indicating that the editing process does not compromise the ability of the ABCs to produce fully differentiated epithelial sheets. Edited ABCs produced epithelial sheets containing basal (KRT5), ciliated (ACT) and secretory cells (MUC5B), further indicating the formation of differentiated epithelial sheets. (D) Representative traces from Ussing chamber analysis showing a non-CF control, an uncorrected CF sample, esDNA PS and esDNA OM corrected CF samples. Uncorrected CF samples show minimal responses to forskolin and CFTRinh-172 which activate and inhibit CFTR, respectively. All edited samples showed restored responses to forskolin and CFTRinh-172 indicating restored CFTR function. (E) Percent restoration of CFTR function in corrected CF samples relative to non-CF controls plotted as a function of allelic correction. Edited samples showed 30-60% restoration of CFTR function on average. (F) Raw CFTRinh-172 short circuit currents observed in CF donor ABCs compared to non-CF controls and uncorrected CF controls (n=3 biological replicates). Samples corrected using esDNA show increased response to CFTRinh-172 relative to uncorrected controls. This change in current was statistically significant when compared using one-way analysis of variance (ANOVA) followed by multiple comparisons test (p<0.05).

FIGS. 6A-6F. esDNA templates improve gene insertion in HSPCs, T-cells and HUVECs but not iPSCs. HR in HSPCs significantly increased with esDNA compared to end-modified ssDNA in the (A) HBB locus (47±22% vs 18±13%), (B) CFTR locus (20±6% vs. 5±5%), and (C) CCR5 locus (38±26% vs. 15±3%). (D) In HUVECs, esDNA increased HR in the CFTR locus from 4±3% obtained with end-modified ssDNA to 12±5% (p=0.0531). (E) In T-cells, esDNA increased HR in the CFTR locus from 13±6% obtained with end-modified ssDNA to 30±10%. (F) Gene insertion with esDNA (30±12%) was significantly lower in iPSCs in the CFTR locus than unmodified ssDNA (71±17%). 3 biological replicates were tested for each cell type. Groups in A-E were compared using a paired T-test. ** and * represent p<0.01, p< and p<0.05 respectively for all panels.

FIG. 7A-7E. Knocking out TREX1 improved Gene Insertion without chemically modifying ssDNA. (A) Percentage of alleles with TREX1 knocked out in non-CF ABCs samples (n=3 biological replicates). (B) HR using unmodified ssDNA templates was improved when TREX1 was knocked out in non-CF ABCs. Knocking out TREX1 did not improve HR by esDNA templates (n=4 biological replicates). (C) Graphic showing additional chemical modification combinations including commercially available proprietary end modifications. (D) Gene insertion with ssDNA using combinations of chemical modifications in ABCs. Both esDNA OM (40±21%) and esDNA PS (34±16%) showed improved gene insertion relative to unmodified (3±3%), end-modified (11±6%) and end-blocked ssDNA (Alt-HDR) (23±9%). All other combinations did not improve on esDNA OM (40±21%) and esDNA PS (34±16%) (n=6 biological replicates). (E) Modification of every 10th base with PS groups also improved gene insertion compared to unmodified or end-modified ssDNA. Gene insertion was reduced when every fourth base was modified. ** and * represent p<0.01, p< and p<0.05 respectively in all panels.

FIG. 8. Gene insertion in exon 11 of the CFTR locus using Cas9 mRNA, sgRNA and ssDNA templates delivered using lipid nanoparticle formulations. Gene insertion was superior when eDNA templates were used in the formulation compared to unmodified SSDNA.

### DETAILED DESCRIPTION

It is to be understood that this disclosure is not limited to particular embodiments described, as such may, of course, vary. It is also to be understood that the terminology used herein is for the purpose of describing particular embodiments only, and is not intended to be limiting, since the scope of this disclosure will be limited only by the appended claims.

The detailed description is divided into various sections only for the reader's convenience and disclosure found in any section may be combined with that in another section. Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated by reference to disclose and describe the methods and/or materials in connection with which the publications are cited.

All numerical designations, e.g., pH, temperature, time, concentration, and molecular weight, including ranges, are approximations which are varied (+) or (−) by increments of 0.1 or 1.0, where appropriate. It is to be understood, although not always explicitly stated, that all numerical designations are preceded by the term “about.” It also is to be understood, although not always explicitly stated, that the reagents described herein are merely exemplary and that equivalents of such are known in the art.

It must be noted that as used herein and in the appended claims, the singular forms “a”, “an”, and “the” include plural referents unless the context clearly dictates otherwise. Thus, for example, reference to “a cell” includes a plurality of cells.

As will be understood by one skilled in the art, for any and all purposes, all ranges disclosed herein also encompass any and all possible subranges and combinations of subranges thereof. Furthermore, as will be understood by one skilled in the art, a range includes each individual member.

The practice of the present disclosure will employ, unless otherwise indicated, conventional techniques of tissue culture, immunology, molecular biology, microbiology, cell biology and recombinant DNA, which are within the skill of the art. See, e.g., Sambrook and Russell eds. (2001) Molecular Cloning: A Laboratory Manual, 3rd edition; the series Ausubel et al. eds. (2007) Current Protocols in Molecular Biology; the series Methods in Enzymology (Academic Press, Inc., N.Y.); MacPherson et al. (1991) PCR 1: A Practical Approach (IRL Press at Oxford University Press); MacPherson et al. (1995) PCR 2: A Practical Approach; Harlow and Lane eds. (1999) Antibodies, A Laboratory Manual; Freshney (2005) Culture of Animal Cells: A Manual of Basic Technique, 5th edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Hames and Higgins eds. (1984) Transcription and Translation; Immobilized Cells and Enzymes (IRL Press (1986)); Perbal (1984) A Practical Guide to Molecular Cloning; Miller and Calos eds. (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Herzenberg et al. eds (1996) Weir's Handbook of Experimental Immunology; Manipulating the Mouse Embryo: A Laboratory Manual, 3rd edition (Cold Spring Harbor Laboratory Press (2002)); Sohail (ed.) (2004) Gene Silencing by RNA Interference: Technology and Application (CRC Press).

### Definitions

As it would be understood, the section or subsection headings as used herein is for organizational purposes only and are not to be construed as limiting and/or separating the subject matter described.

The practice of the present disclosure employs, unless otherwise indicated, conventional techniques of immunology, biochemistry, chemistry, molecular biology, microbiology, cell biology, genomics and recombinant DNA, which are within the skill of the art. See Sambrook, Fritsch and Maniatis, MOLECULAR CLONING: A LABORATORY MANUAL, 2nd edition (1989); CURRENT PROTOCOLS IN MOLECULAR BIOLOGY (F. M. Ausubel, et al. eds., (1987)); the series METHODS IN ENZYMOLOGY (Academic Press, Inc.): PCR 2: A PRACTICAL APPROACH (M. J. MacPherson, B. D. Hames and G. R. Taylor eds. (1995)), Harlow and Lane, eds. (1988) ANTIBODIES, A LABORATORY MANUAL, and ANIMAL CELL CULTURE (R.I. Freshney, ed. (1987)).

The term “about” when used before a numerical designation, e.g., temperature, time, amount, concentration, and such other, including a range, indicates approximations which may vary by (+) or (−) 10%, 5% or 1%.

“Comprising” or “comprises” is intended to mean that the compositions, for example media, and methods include the recited elements, but not excluding others. “Consisting essentially of” when used to define compositions and methods, shall mean excluding other elements of any essential significance to the combination for the stated purpose. Thus, a composition consisting essentially of the elements as defined herein would not exclude other materials or steps that do not materially affect the basic and novel characteristic(s) of the claimed disclosure. “Consisting of” shall mean excluding more than trace elements of other ingredients and substantial method steps. Embodiments defined by each of these transition terms are within the scope of this disclosure.

As used herein, comparative terms as used herein, such as high, low, increase, decrease, reduce, or any grammatical variation thereof, can refer to certain variation from the reference. In some embodiments, such variation can refer to about 10%, or about 20%, or about 30%, or about 40%, or about 50%, or about 60%, or about 70%, or about 80%, or about 90%, or about 1 fold, or about 2 folds, or about 3 folds, or about 4 folds, or about 5 folds, or about 6 folds, or about 7 folds, or about 8 folds, or about 9 folds, or about 10 folds, or about 20 folds, or about 30 folds, or about 40 folds, or about 50 folds, or about 60 folds, or about 70 folds, or about 80 folds, or about 90 folds, or about 100 folds or more higher than the reference. In some embodiments, such variation can refer to about 1%, or about 2%, or about 3%, or about 4%, or about 5%, or about 6%, or about 7%, or about 8%, or about 0%, or about 10%, or about 20%, or about 30%, or about 40%, or about 50%, or about 60%, or about 70%, or about 75%, or about 80%, or about 85%, or about 90%, or about 95%, or about 96%, or about 97%, or about 98%, or about 99% of the reference.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

“Substantially” or “essentially” means nearly totally or completely, for instance, 95% or greater of some given quantity. In some embodiments, “substantially” or “essentially” means 95%, 96%, 97%, 98%, 99%, 99.5%, or 99.9%.

The terms or “acceptable,” “effective,” or “sufficient” when used to describe the selection of any components, ranges, dose forms, etc. disclosed herein intend that said component, range, dose form, etc. is suitable for the disclosed purpose.

The term “subject,” “host,” “individual,” and “patient” are as used interchangeably herein to refer to animals, typically mammalian animals. Any suitable mammal can be treated by a method described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments, a mammal is a human. A mammal can be any age or at any stage of development (e.g., an adult, teen, child, infant, or a mammal in utero). A mammal can be male or female. In some embodiments, a subject is a human. In some embodiments, a subject has, or is diagnosed of having, or is suspected of having, or is at risk of having a disease, such as a cancer or a hereditary disease such as sickle cell anemia or cystic fibrosis.

The term “isolated” as used herein with respect to nucleic acids, such as DNA or RNA, refers to molecules separated from other DNAs or RNAs, respectively that are present in the natural source of the macromolecule. The term “isolated peptide fragment” is meant to include peptide fragments which are not naturally occurring as fragments and would not be found in the natural state. The term “isolated” is also used herein to refer to polypeptides and proteins that are isolated from other cellular proteins and is meant to encompass both purified and recombinant polypeptides. In other embodiments, the term “isolated” means separated from constituents, cellular and otherwise, in which the cell, tissue, polynucleotide, peptide, polypeptide, protein, antibody or fragment(s) thereof, which are normally associated in nature. For example, an isolated cell is a cell that is separated form tissue or cells of dissimilar phenotype or genotype. As is apparent to those of skill in the art, a non-naturally occurring polynucleotide, peptide, polypeptide, protein, antibody or fragment(s) thereof, does not require “isolation” to distinguish it from its naturally occurring counterpart.

“Cell,” “host cell” or “recombinant host cell” are terms used interchangeably herein. It is understood that such terms refer not only to the particular subject cell but to the progeny or potential progeny of such a cell. The cells can be of any one or more of the type murine, rat, rabbit, simian, bovine, ovine, porcine, canine, feline, equine, and primate, particularly human. Because certain modifications may occur in succeeding generations due to either mutation or environmental influences, such progeny may not, in fact, be identical to the parent cell, but are still included within the scope of the term as used herein.

The term “stem cell” refers to a cell that is in an undifferentiated or partially differentiated state and has the capacity for self-renew and to generate differentiated progeny. Self-renewal is defined as the capability of a stem cell to proliferate and give rise to more such stem cells, while maintaining its developmental potential (i.e., totipotent, pluripotent, multipotent, etc.). The term “progenitor stem cells,” as used herein, refers to cells with the ability to self-renew and to differentiate into other mature or less mature phenotypes which include, but are not limited to, multipotent mesenchymal stromal cells, adventitial reticular cells, vascular pericytes, fibroblasts and bone-lining cells. The term “somatic stem cell” is used herein to refer to any stem cell derived from non-embryonic tissue, including fetal, juvenile, and adult tissue. Natural somatic stem cells have been isolated from a wide variety of adult tissues including blood, bone marrow, brain, olfactory epithelium, skin, pancreas, skeletal muscle, and cardiac muscle. Exemplary naturally occurring somatic stem cells include, but are not limited to, mesenchymal stem cells (MSCs) and neural stem cells (NSCs). In some embodiments, the stem or progenitor cells can be embryonic stem cells. As used herein, “embryonic stem cells” refers to stem cells derived from tissue formed after fertilization but before the end of gestation, including pre-embryonic tissue (such as, for example, a blastocyst), embryonic tissue, or fetal tissue taken any time during gestation, typically but not necessarily before approximately 10-12 weeks gestation. Most frequently, embryonic stem cells are pluripotent cells derived from the early embryo or blastocyst. Embryonic stem cells can be obtained directly from suitable tissue, including, but not limited to human tissue, or from established embryonic cell lines. “Embryonic-like stem cells” refer to cells that share one or more, but not all characteristics, of an embryonic stem cell. In one aspect, the term “stem cell” excludes embryonic stem cells that have been generated from a non-renewable source.

As used herein, the phrase “hematopoietic stem cell” or “Hematopoietic Stem and Progenitor cell (“HSPC”)” refers to a cell with the ability to reconstitute through multiple differentiation steps all lineages present in the immune system such as erythrocytes, granulocytes, monocytes, mast cells, lymphocytes and megakaryocytes. When cultured under the appropriate conditions, hematopoietic stem cells are self-renewing and have the capacity to maintain their pluripotency. In one aspect, hematopoietic stem cells are CD34+ cells.

As used herein, the term “HUVEC cell” refers to a human umbilical vein endothelial cell isolated from the vein of an umbilical cord. HUVEC cells can be obtained from ATCC (Catalog number: CRL-1730, atcc.org/products/crl-1730, last accessed Apr. 6, 2024), Thermo Fisher (Catalog number: C0035C, thermofisher.com/order/catalog/product/C0035C? SID=srch-hj-C0035C, last accessed Apr. 6, 2024), Cell Applications (Catalog Number: 200-05n, cellapplications.com/human-umbilical-vein-endothelial-cells-huvec, last accessed Apr. 6, 2024.

As used herein, “treating” or “treatment” of a disease in a subject refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of this technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. In one aspect, treatment excludes prophylaxis.

An “effective amount” is an amount sufficient to effect beneficial or desired results. An effective amount can be administered in one or more administrations, applications or dosages. Such delivery is dependent on a number of variables including the time period for which the individual dosage unit is to be used, the bioavailability of the therapeutic agent, the route of administration, etc. It is understood, however, that specific dose levels of the therapeutic agents of the present disclosure for any particular subject depends upon a variety of factors including the activity of the specific compound employed, the age, body weight, general health, sex, and diet of the subject, the time of administration, the rate of excretion, the drug combination, and the severity of the particular disorder being treated and form of administration. Treatment dosages generally may be titrated to optimize safety and efficacy. Typically, dosage-effect relationships from in vitro and/or in vivo tests initially can provide useful guidance on the proper doses for patient administration. In general, one will desire to administer an amount of the composition or cell to provide the therapeutic benefit in vitro or in vivo by at least 10%, 25%, 40%, 60%, 80%, 90% or 95% as compared to control. Determination of these parameters is well within the skill of the art. These considerations, as well as effective formulations and administration procedures are well known in the art and are described in standard textbooks.

Administration or delivery in vivo can be effected in one dose, continuously or intermittently throughout the course of treatment. Methods of determining the most effective means and dosage of administration are well known to those of skill in the art and will vary with the composition used for therapy, the purpose of the therapy, and the subject being treated. Single or multiple administrations can be carried out with the dose level and pattern being selected by the treating physician.

The pharmaceutical compositions can be administered by inhalation, orally, intranasally, parenterally, injection, orally and may take the form of tablets, lozenges, granules, capsules, pills, ampoules, suppositories or aerosol form. They may also take the form of suspensions, solutions and emulsions of the active ingredient in aqueous or nonaqueous diluents, syrups, granulates or powders. In addition to an agent of the present disclosure, the compositions can also contain other pharmaceutically active compounds or a plurality of systems or cells of the disclosure.

More particularly, an agent of the present disclosure also referred to herein as the active ingredient, may be administered for therapy by any suitable route including oral, rectal, nasal, topical (including transdermal, aerosol, buccal and sublingual), vaginal, parenteral (including subcutaneous, intramuscular, intravenous and intradermal) and pulmonary. It will also be appreciated that the preferred route will vary with the condition and age of the recipient, and the disease being treated.

While it is possible for the agent to be administered alone, it is preferable to present it as a pharmaceutical formulation comprising, or consisting essentially of, or consisting of at least one active ingredient, as defined above, together with one or more pharmaceutically acceptable carriers therefor and optionally other therapeutic agents. Each carrier must be “acceptable” in the sense of being compatible with the other ingredients of the formulation and not injurious to the patient.

Formulations include those suitable for oral, rectal, nasal, topical (including transdermal, buccal and sublingual), vaginal, parenteral (including subcutaneous, intramuscular, intravenous and intradermal) and pulmonary administration. The formulations may conveniently be presented in unit dosage form and may be prepared by any methods well known in the art of pharmacy. Such methods include the step of bringing into association the active ingredient with the carrier that constitutes one or more accessory ingredients. In general, the formulations are prepared by uniformly and intimately bringing into association the active ingredient with liquid carriers or finely divided solid carriers or both, and then if necessary, shaping the product.

Formulations of the present disclosure suitable for oral administration may be presented as discrete units such as capsules, cachets or tablets, each containing a predetermined amount of the active ingredient; as a powder or granules; as a solution or suspension in an aqueous or non-aqueous liquid; or as an oil-in-water liquid emulsion or a water-in-oil liquid emulsion. The active ingredient may also be presented as a bolus, electuary or paste.

Formulations suitable for topical administration to the eye also include eye drops wherein the active ingredient is dissolved or suspended in a suitable carrier, especially an aqueous solvent for the agent.

Formulations for rectal administration may be presented as a suppository with a suitable base comprising, for example, cocoa butter or a salicylate. Formulations suitable for vaginal administration may be presented as pessaries, tampons, creams, gels, pastes, foams or spray formulations containing in addition to the agent, such carriers as are known in the art to be appropriate.

Formulations suitable for nasal administration or aerosol (directly into the lung), wherein the carrier is a solid, include a coarse powder having a particle size, for example, in the range of about 20 to about 500 microns which is administered as a dry powder or in an inhaler device by rapid inhalation through the nasal passage from a container of the powder held close up to the nose. Suitable formulations wherein the carrier is a liquid for administration as, for example, nasal spray, nasal drops, or by aerosol administration by nebulizer, include aqueous or oily solutions of the agent.

Formulations suitable for parenteral administration include aqueous and non-aqueous isotonic sterile injection solutions which may contain anti-oxidants, buffers, bacteriostats and solutes which render the formulation isotonic with the blood of the intended recipient; and aqueous and non-aqueous sterile suspensions which may include suspending agents and thickening agents, and liposomes or other microparticulate systems which are designed to target the compound to blood components or one or more organs. The formulations may be presented in unit-dose or multi-dose sealed containers, for example, ampoules and vials, and may be stored in a freeze-dried (lyophilized) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the ingredients particularly mentioned above, the formulations of this disclosure may include other agents conventional in the art having regard to the type of formulation in question, for example, those suitable for oral administration may include such further agents as sweeteners, thickeners and flavoring agents. It also is intended that the agents, compositions and methods of this disclosure be combined with other suitable compositions and therapies.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides, ribonucleotides, hybrid polynucleotides or analogs thereof. Polynucleotides may have any three-dimensional structure, and may perform any function, known or unknown. The following are non-limiting examples of polynucleotides: coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. The term also encompasses nucleic-acid-like structures with synthetic backbones. A polynucleotide may comprise one or more modified nucleotides, such as methylated nucleotides and nucleotide analogs or those as described herein. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

“Nucleic acid analog” refers to modified nucleic acids or species unrelated to nucleic acids that are capable of providing selective binding to nucleic acids or other nucleic acid analogs. As used herein, the term “nucleotide analogs” (e.g., an A, G, T, C or U analog) includes nucleic acids where the internucleotide phosphodiester bond of DNA or RNA is modified to enhance bio-stability of the oligomer and “tune” the selectivity/specificity for target molecules. Such modifications may include and are not limited to phosphorothioates, phosphorodithioates, phosphotriesters, phosphoramidates or methylphosphonates. The 2′-O-methyl, allyl and 2′-deoxy-2′-fluoro RNA analogs, when incorporated into an oligomer show increased biostability and stabilization of the RNA/DNA duplex (Lesnik et al., (1993), Biochemistry, 32:7832). As used herein, the term “nucleic acid analogs” also include alpha anomers (α-DNA), L-DNA (mirror image DNA), 2′-5′ linked RNA, branched DNA/RNA or chimeras of natural DNA or RNA and the above-modified nucleic acids. For the purposes of the present disclosure, any nucleic acid containing a “nucleotide analog” shall be considered as a nucleic acid analog. Backbone replaced nucleic acid analogs can also be adapted to for use as immobilized selective moieties of the present invention. For purposes of the present disclosure, the peptide nucleic acids (PNAs) (Nielsen et al., (1993), Anti-Cancer Drug Design, 8:53; Engels et al., (1992), Angew, Chem. Int. Ed. Eng., 31:1008) and carbamate-bridged morpholino-type oligonucleotide analogs (Burger, D. R., (1993), J. Clinical Immunoassay, 16:224; Uhlmann, et al., (1993), Methods in Molecular Biology, 20. “Protocols for Oligonucleotides and Analogs,” ed. Sudhir Agarwal, Humana Press, NJ, U.S.A., pp. 335-389) are also embraced by the term “nucleic acid analogs”. Both exhibit sequence-specific binding to DNA with the resulting duplexes being more thermally stable than the natural DNA/DNA duplex. Other backbone-replaced nucleic acids are well known to those skilled in the art and can also be used in the present invention (See, e.g., Uhlmann et al., (1993), Methods in Molecular Biology, 20, “Protocols for Oligonucleotides and Analogs,” ed. Sudhir Agrawal, Humana Press, NJ, U.S.A., pp. 335).

As used herein the term “wild type” is a term of the art understood by skilled persons and means the typical form of an organism, strain, gene or characteristic as it occurs in nature as distinguished from mutant or variant forms.

As used herein the term “variant” should be taken to mean the exhibition of qualities that have a pattern that deviates from what occurs in nature.

The terms “non-naturally occurring” or “engineered” are used interchangeably and indicate the involvement of the hand of man. The terms, when referring to nucleic acid molecules or polypeptides mean that the nucleic acid molecule or the polypeptide is at least substantially free from at least one other component with which they are naturally associated in nature and as found in nature.

“Complementarity” refers to the ability of a nucleic acid to form hydrogen bond(s) with another nucleic acid sequence by either traditional Watson-Crick base pairing or other non-traditional types. A percent complementarity indicates the percentage of residues in a nucleic acid molecule which can form hydrogen bonds (e.g., Watson-Crick base pairing) with a second nucleic acid sequence (e.g., 5, 6, 7, 8, 9, 10 out of 10 being 50%, 60%, 70%, 80%, 90%, and 100% complementary). “Perfectly complementary” means that all the contiguous residues of a nucleic acid sequence will hydrogen bond with the same number of contiguous residues in a second nucleic acid sequence. “Substantially complementary” as used herein refers to a degree of complementarity that is at least 60%, 65%, 70%, 75%, 80%, 85%, 90%, 95%, 97%, 98%, 99%, or 100% over a region of 8, 9, 10, 11, 12, 13, 14, 15, 16, 17, 18, 19, 20, 21, 22, 23, 24, 25, 30, 35, 40, 45, 50, or more nucleotides, or refers to two nucleic acids that hybridize under stringent conditions.

As used herein, “stringent conditions” for hybridization refer to conditions under which a nucleic acid having complementarity to a target sequence predominantly hybridizes with the target sequence, and substantially does not hybridize to non-target sequences. Stringent conditions are generally sequence-dependent, and vary depending on a number of factors. In general, the longer the sequence, the higher the temperature at which the sequence specifically hybridizes to its target sequence. Non-limiting examples of stringent conditions are described in detail in Tijssen (1993), Laboratory Techniques In Biochemistry And Molecular Biology-Hybridization With Nucleic Acid Probes Part I, Second Chapter “Overview of principles of hybridization and the strategy of nucleic acid probe assay”, Elsevier, N.Y. Where reference is made to a polynucleotide sequence, then complementary or partially complementary sequences are also envisaged. These are preferably capable of hybridizing to the reference sequence under highly stringent conditions. Generally, in order to maximize the hybridization rate, relatively low-stringency hybridization conditions are selected: about 20 to 25° C. lower than the thermal melting point (Tₘ). The Tₘ is the temperature at which 50% of specific target sequence hybridizes to a perfectly complementary probe in solution at a defined ionic strength and pH. Generally, in order to require at least about 85% nucleotide complementarity of hybridized sequences, highly stringent washing conditions are selected to be about 5 to 15° C. lower than the Tₘ. In order to require at least about 70% nucleotide complementarity of hybridized sequences, moderately-stringent washing conditions are selected to be about 15 to 30° C. lower than the Tₘ. Highly permissive (very low stringency) washing conditions may be as low as 50° C. below the Tₘ, allowing a high level of mis-matching between hybridized sequences. Those skilled in the art will recognize that other physical and chemical parameters in the hybridization and wash stages can also be altered to affect the outcome of a detectable hybridization signal from a specific level of homology between target and probe sequences. Exemplary highly stringent conditions comprise incubation in 50% formamide, 5×SSC, and 1% SDS at 42° C., or incubation in 5×SSC and 1% SDS at 65° C., with wash in 0.2×SSC and 0.1% SDS at 65° C.

“Hybridization” refers to a reaction in which one or more polynucleotides react to form a complex that is stabilized via hydrogen bonding between the bases of the nucleotide residues. The hydrogen bonding may occur by Watson Crick base pairing, Hoogstein binding, or in any other sequence specific manner. The complex may comprise two strands forming a duplex structure, three or more strands forming a multi stranded complex, a single self-hybridizing strand, or any combination of these. A hybridization reaction may constitute a step in a more extensive process, such as the initiation of PCR, or the cleavage of a polynucleotide by an enzyme. A sequence capable of hybridizing with a given sequence is referred to as the “complement” of the given sequence.

As used herein, the term “genomic locus” or “locus” (plural loci) is the specific location of a gene or DNA sequence on a chromosome. A “gene” refers to stretches of DNA or RNA that encode a polypeptide or an RNA chain that has functional role to play in an organism and hence is the molecular unit of heredity in living organisms. For the purpose of this disclosure it may be considered that genes include regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites and locus control regions.

As used herein, “expression of a genomic locus” or “gene expression” is the process by which information from a gene is used in the synthesis of a functional gene product. The products of gene expression are often proteins, but in non-protein coding genes such as IRNA genes or tRNA genes, the product is functional RNA. The process of gene expression is used by all known life-eukaryotes (including multicellular organisms), prokaryotes (bacteria and archaea) and viruses to generate functional products to survive. As used herein “expression” of a gene or nucleic acid encompasses not only cellular gene expression, but also the transcription and translation of nucleic acid(s) in cloning systems and in any other context. As used herein, “expression” also refers to the process by which a polynucleotide is transcribed from a DNA template (such as into and mRNA or other RNA transcript) and/or the process by which a transcribed mRNA is subsequently translated into peptides, polypeptides, or proteins. Transcripts and encoded polypeptides may be collectively referred to as “gene product.” If the polynucleotide is derived from genomic DNA, expression may include splicing of the mRNA in a eukaryotic cell.

As used herein, the term “nuclease” refers to an exonuclease or an endonuclease. Endonucleases are enzymes that cleave or hydrolyze the bond between nucleotides in an RNA or DNA molecules. In some embodiments, the endonuclease is selected from FokI, AarI, AceIII, AcH, AloI, Bael, Bbrll, CdH, QePl, Edl, Esp3l, Finl, Mbol, sapl, or SspD51. In some embodiments, the endonuclease is FokI, naturally found in Flavobacterium okeanokoites.

As used herein, the phrase “nuclease catalytic domain” refers to a protein domain comprising the active site of an endonuclease or an exonuclease enzyme. Such nuclease catalytic domain can be, for instance, a “cleavage domain” or a “nickase domain”. The phrase “cleavage domain” refers to a protein domain whose catalytic activity generates a Double Strand Break (DSB) in a DNA target. The phrase “nickase domain” refers to a protein domain whose catalytic activity generates a single strand break in a DNA target sequence.

The terms “polypeptide”, “peptide” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. As used herein the term “amino acid” includes natural and/or unnatural or synthetic amino acids, including glycine and both the D or L optical isomers, and amino acid analogs and peptidomimetics.

As used herein, the term “domain” or “protein domain” refers to a part of a protein sequence that may exist and function independently of the rest of the protein chain.

As described in aspects of this disclosure, sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences.

The term “regulatory element” is intended to include promoters, enhancers, internal ribosomal entry sites (IRES), and other expression control elements (e.g., transcription termination signals, such as polyadenylation signals and poly-U sequences). Such regulatory elements are described, for example, in Goeddel, GENE EXPRESSION TECHNOLOGY: METHODS IN ENZYMOLOGY 185, Academic Press, San Diego, Calif. (1990). Regulatory elements include those that direct constitutive expression of a nucleotide sequence in many types of host cell and those that direct expression of the nucleotide sequence only in certain host cells (e.g., tissue-specific regulatory sequences). A tissue-specific promoter may direct expression primarily in a desired tissue of interest, such as muscle, neuron, bone, skin, blood, specific organs (e.g. liver, pancreas), or particular cell types (e.g. lymphocytes). Regulatory elements may also direct expression in a temporal-dependent manner, such as in a cell-cycle dependent or developmental stage-dependent manner, which may or may not also be tissue or cell-type specific. In some embodiments, a vector comprises one or more pol III promoter (e.g., 1, 2, 3, 4, 5, or more pol III promoters), one or more pol II promoters (e.g., 1, 2, 3, 4, 5, or more pol II promoters), one or more pol I promoters (e.g., 1, 2, 3, 4, 5, or more pol I promoters), or combinations thereof. Examples of pol III promoters include, but are not limited to, U6 and H1 promoters. Examples of pol II promoters include, but are not limited to, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the β-actin promoter, the phosphoglycerol kinase (PGK) promoter, and the EF1α promoter. Also encompassed by the term “regulatory element” are enhancer elements, such as WPRE; CMV enhancers; the R-U5′ segment in LTR of HTLV-I (Mol. Cell. Biol., Vol. 8 (1), p. 466-472, 1988); SV40 enhancer; and the intron sequence between exons 2 and 3 of rabbit β-globin (Proc. Natl. Acad. Sci. USA., Vol. 78 (3), p. 1527-31, 1981). It will be appreciated by those skilled in the art that the design of the expression vector can depend on such factors as the choice of the host cell to be transformed, the level of expression desired, etc. A vector can be introduced into host cells to thereby produce transcripts, proteins, or peptides, including fusion proteins or peptides, encoded by nucleic acids as described herein (e.g., gene-editing system transcripts, proteins, enzymes, mutant forms thereof, fusion proteins thereof, etc.). With regards to regulatory sequences, mention is made of U.S. Patent No. U.S. Pat. No. 7,776,321 B2, the contents of which are incorporated by reference herein in their entirety. With regards to promoters, mention is made of PCT publication WO 2011/028929 and U.S. Patent Publ. No.: 2011/0027239 A1, the contents of which are incorporated by reference herein in their entirety.

Vectors can be designed for expression of gene-editing system transcripts (e.g. nucleic acid transcripts, proteins, or enzymes) in prokaryotic or eukaryotic cells. For example, gene-editing system transcripts can be expressed in bacterial cells such as Escherichia coli, insect cells (using baculovirus expression vectors), yeast cells, or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Vectors may be introduced and propagated in a prokaryote or prokaryotic cell. In some embodiments, a prokaryote is used to amplify copies of a vector to be introduced into a eukaryotic cell or as an intermediate vector in the production of a vector to be introduced into a eukaryotic cell (e.g. amplifying a plasmid as part of a viral vector packaging system). In some embodiments, a prokaryote is used to amplify copies of a vector and express one or more nucleic acids, such as to provide a source of one or more proteins for delivery to a host cell or host organism. Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, such as to the amino terminus of the recombinant protein. Such fusion vectors may serve one or more purposes, such as: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Example fusion expression vectors include pGEX (Pharmacia Biotech Inc; Smith and Johnson, 1988. Gene 67:31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E. coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

In some embodiments, a vector is a yeast expression vector. Examples of vectors for expression in yeast Saccharomyces cerevisiae include pYepSec1 (Baldari, et al., 1987. EMBO J. 6:229-234), pMFa (Kuijan and Herskowitz, 1982. Cell 30:933-943), pJRY88 (Schultz et al., 1987. Gene 54:113-123), pYES2 (Invitrogen Corporation, San Diego, Calif.), and picZ (In Vitrogen Corp, San Diego, Calif.).

In some embodiments, a vector drives protein expression in insect cells using baculovirus expression vectors. Baculovirus vectors available for expression of proteins in cultured insect cells (e.g., SF9 cells) include the pAc series (Smith, et al., 1983. Mol. Cell. Biol. 3:2156-2165) and the pVL series (Lucklow and Summers, 1989. Virology 170:31-39).

In some embodiments, a vector is capable of driving expression of one or more sequences in mammalian cells using a mammalian expression vector. Examples of mammalian expression vectors include pCDM8 (Seed, 1987. Nature 329:840) and pMT2PC (Kaufman, et al., 1987. EMBO J. 6:187-195). When used in mammalian cells, the expression vector's control functions are typically provided by one or more regulatory elements. For example, commonly used promoters are derived from polyoma, adenovirus 2, cytomegalovirus, simian virus 40, and others disclosed herein and known in the art. For other suitable expression systems for both prokaryotic and eukaryotic cells see, e.g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In some embodiments, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1:268-277), lymphoid-specific promoters (Calame and Eaton, 1988. Adv. Immunol. 43:235-275), in particular promoters of T cell receptors (Winoto and Baltimore, 1989. EMBO J. 8:729-733) and immunoglobulins (Baneiji, et al., 1983. Cell 33:729-740; Queen and Baltimore, 1983. Cell 33:741-748), neuron-specific promoters (e.g., the neurofilament promoter; Byrne and Ruddle, 1989. Proc. Natl. Acad. Sci. USA 86:5473-5477), pancreas-specific promoters (Edlund, et al., 1985. Science 230:912-916), and mammary gland-specific promoters (e.g., milk whey promoter; U.S. Pat. No. 4,873,316 and European Application Publication No. 264,166). Developmentally-regulated promoters are also encompassed, e.g., the murine hox promoters (Kessel and Gruss, 1990. Science 249:374-379) and the α-fetoprotein promoter (Campes and Tilghman, 1989. Genes Dev. 3:537-546). With regards to these prokaryotic and eukaryotic vectors, mention is made of U.S. Pat. No. 6,750,059, the contents of which are incorporated by reference herein in their entirety. Other embodiments of the disclosure may relate to the use of viral vectors, with regards to which mention is made of U.S. patent application Ser. No. 13/092,085 (published as US Patent Publ. No.: 20120003201A1), the contents of which are incorporated by reference herein in their entirety. Tissue-specific regulatory elements are known in the art and in this regard, mention is made of U.S. Pat. No. 7,776,321, the contents of which are incorporated by reference herein in their entirety.

In some embodiments, a regulatory element is operably linked to one or more elements of a gene-editing system so as to drive expression of the one or more elements of the gene-editing system.

The gene-editing systems described herein can further comprise one or more labels or detection tags (e.g., FLAG™ tag, epitope or protein tags, such as myc tag, 6 His, and fluorescent fusion protein). In an aspect, the label (e.g., FLAG™ tag) is fused to the NLS. In an aspect, the disclosed methods and compositions further comprise a fusion protein, or a polynucleotide encoding the same. In various aspects, the fusion protein comprises at least one epitope-providing amino acid sequence (e.g., “epitope-tag”), wherein the epitope-tag is selected from i) an epitope-tag added to the N- and/or C-terminus of a protein, or ii) an epitope-tag inserted into a region of a protein, and an epitope-tag replacing a number of amino acids in a protein.

As used herein, “epitope tags” refer to short stretches of amino acids to which a specific antibody can be raised, which in some aspects allows one to specifically identify and track the tagged protein that has been added to a living organism or to cultured cells. Detection of the tagged molecule can be achieved using a number of different techniques. Examples of such techniques include immunohistochemistry, immunoprecipitation, flow cytometry, immunofluorescence microscopy, ELISA, immunoblotting (“Western blotting”), and affinity chromatography. Epitope tags add a known epitope (e.g., antibody binding site) on the subject protein, to provide binding of a known and often high-affinity antibody, and thereby allowing one to specifically identify and track the tagged protein that has been added to a living organism or to cultured cells. Examples of epitope tags include, but are not limited to, myc, T7, GST, GFP, HA (hemagglutinin), V5 and FLAG tags. The first four examples are epitopes derived from existing molecules. In contrast, FLAG is a synthetic epitope tag designed for high antigenicity (see, e.g., U.S. Pat. Nos. 4,703,004 and 4,851,341). Epitope tags can have one or more additional functions, beyond recognition by an antibody.

In an aspect, the disclosed methods and compositions comprise an epitope-tag wherein the epitope-tag has a length of between 6 to 15 amino acids. In an alternative aspect, the epitope-tag has a length of 9 to 11 amino acids. The disclose methods and compositions can also comprise a fusion protein comprising two or more epitope-tags, either spaced apart or directly in tandem. Further, the disclosed methods and composition can comprise 2, 3, 4, 5 or even more epitope-tags, as long as the fusion protein maintains its biological activity/activities (e.g., “functional”).

In an aspect, the epitope-tag is a VSV-G tag, CD tag, calmodulin-binding peptide tag, S-tag, Avitag, SF-TAP-tag, strep-tag, myc-tag, FLAG-tag, T7-tag, HA (hemagglutinin)-tag, His-tag, S-tag, GST-tag, or GFP-tag. The sequences of these tags are described in the literature and well known to the person of skill in art.

A “composition” is intended to mean a combination of active polypeptide, polynucleotide or antibody and another compound or composition, inert (e.g., a detectable label) or active (e.g., a gene delivery vehicle) alone or in combination with a carrier which can in one embodiment be a simple carrier like saline or pharmaceutically acceptable or a solid support as defined below.

A “pharmaceutical composition” is intended to include the combination of an active polypeptide, polynucleotide or antibody with a carrier, inert or active such as a solid support, making the composition suitable for diagnostic or therapeutic use in vitro, in vivo or ex vivo.

As used herein, the term “pharmaceutically acceptable carrier” encompasses any of the standard pharmaceutical carriers, such as a phosphate buffered saline solution, water, and emulsions, such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton).

As used herein, the term “detectably labeled” means that the agent (biologic or small molecule) is attached to another molecule, compound or polymer that facilitates detection of the presence of the agent in vitro or in vivo. A “detectable label” intends a directly or indirectly detectable compound or composition that is conjugated directly or indirectly to the composition to be detected, e.g., N-terminal histidine tags (N-His), magnetically active isotopes, e.g., ¹¹⁵Sn, ¹¹⁷Sn and ¹¹⁹Sn, a non-radioactive isotopes such as ¹³C and ¹⁵N, polynucleotide or protein such as an antibody so as to generate a “labeled” composition. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, luminescent compounds, dyes, and proteins, including enzymes. The label may be simply detected, or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component.

Examples of luminescent labels that produce signals include but are not limited to bioluminescence and chemiluminescence. Detectable luminescence response generally comprises a change in, or an occurrence of, a luminescence signal. Suitable methods and luminophores for luminescent labeling assay components are known in the art and described for example in Haugland, Richard P. (1996) Handbook of Fluorescent Probes and Research Chemicals (6th ed.). Examples of luminescent probes include, but are not limited to, aequorin and luciferases.

Examples of suitable fluorescent labels include, but are not limited to, fluorescein, rhodamine, tetramethyl rhodamine, eosin, erythrosin, coumarin, methyl-coumarins, pyrene, Malachite green, stilbene, Lucifer Yellow, Cascade Blue™, and Texas Red. Other suitable optical dyes are described in the Haugland, Richard P. (1996) Handbook of Fluorescent Probes and Research Chemicals (6^{th} ed.).

As used herein, the term “lipid nanoparticle” or “LNP” refers to a nanoparticle comprising one or more lipids. In some embodiments, the LNP has a size of about 500 nm or less, about 450 nm or less, about 400 nm or less, about 350 nm or less, about 300 nm or less, about 250 nm or less, about 200 nm or less, about 150 nm or less, or about 100 nm or less. In some embodiments, the LNP has a size ranging from about 1 nm to about 100 nm.

LNPs for use as delivery vehicles disclosed herein comprise an (i) ionizable lipid; (ii) sterol or other structural lipid; (iii) a non-cationic helper lipid or phospholipid; and optionally a (iv) a polyethylene glycol (PEG) lipid.

In some embodiments, polynucleotides of the present disclosure are formulated as lipid nanoparticle (LNP) compositions. Lipid nanoparticles typically comprise amino lipid, phospholipid, structural lipid and PEG lipid components along with the nucleic acid cargo of interest. The lipid nanoparticles of the invention can be generated using components, compositions, and methods as are generally known in the art, see for example PCT/US2016/052352; PCT/US2016/068300; PCT/US2017/037551; PCT/US2015/027400; PCT/US2016/047406; PCT/US2016000129; PCT/US2016/014280; PCT/US2016/014280; PCT/US2017/038426; PCT/US2014/027077; PCT/US2014/055394; PCT/US2016/52117; PCT/US2012/069610; PCT/US2017/027492; PCT/US2016/059575; PCT/US2016/069491; PCT/US2016/069493; and PCT/US2014/66242, all of which are incorporated by reference herein in their entirety.

In some embodiments, the lipid nanoparticle comprises a molar ratio of 20-60% amino lipid relative to the other lipid components. In some embodiments, the lipid nanoparticle comprises a molar ratio of 20-50%, 20-40%, 20-30%, 30-60%, 30-50%, 30-40%, 40-60%, 40-50%, or 50-60% amino lipid. In some embodiments, the lipid nanoparticle comprises a molar ratio of 20%, 30%, 40%, 50, or 60% amino lipid.

In some embodiments, the lipid nanoparticle comprises a molar ratio of 5-25% phospholipid relative to the other lipid components. In some embodiments, the lipid nanoparticle comprises a molar ratio of 5-30%, 5-15%, 5-10%, 10-25%, 10-20%, 10-25%, 15-25%, 15-20%, 20-25%, or 25-30% phospholipid. In some embodiments, the lipid nanoparticle comprises a molar ratio of 5%, 10%, 15%, 20%, 25%, or 30% non-cationic lipid.

In some embodiments, the lipid nanoparticle comprises a molar ratio of 25-55% structural lipid relative to the other lipid components. In some embodiments, the lipid nanoparticle comprises a molar ratio of 10-55%, 25-50%, 25-45%, 25-40%, 25-35%, 25-30%, 30-55%, 30-50%, 30-45%, 30-40%, 30-35%, 35-55%, 35-50%, 35-45%, 35-40%, 40-55%, 40-50%, 40-45%, 45-55%, 45-50%, or 50-55% structural lipid. In some embodiments, the lipid nanoparticle comprises a molar ratio of 10%, 15%, 20%, 25%, 30%, 35%, 40%, 45%, 50%, or 55% structural lipid.

In some embodiments, the lipid nanoparticle comprises a molar ratio of 0.5-15% PEG lipid relative to the other lipid components. In some embodiments, the lipid nanoparticle comprises a molar ratio of 0.5-10%, 0.5-5%, 1-15%, 1-10%, 1-5%, 2-15%, 2-10%, 2-5%, 5-15%, 5-10%, or 10-15% PEG lipid. In some embodiments, the lipid nanoparticle comprises a molar ratio of 0.5%, 1%, 2%, 3%, 4%, 5%, 6%, 7%, 8%, 9%, 10%, 11%, 12%, 13%, 14%, or 15% PEG-lipid.

In some embodiments, the lipid nanoparticle comprises a molar ratio of 20-60% amino lipid, 5-25% phospholipid, 25-55% structural lipid, and 0.5-15% PEG lipid. In some embodiments, the lipid nanoparticle comprises a molar ratio of 20-60% amino lipid, 5-30% phospholipid, 10-55% structural lipid, and 0.5-15% PEG lipid.

As used herein, the term “TREX1” refers to “Three-prime repair exonuclease 1” (gene name TREX-1), also called Deoxyribonuclease III or DNase III, an enzyme that degrades deoxyribonucleic acid by cleaving the 3′ terminal base of a DNA polymer. TREX1 can digest single-stranded and double-stranded DNA containing a mismatched 3′ overhang. TREX1 is an endoplasmic reticulum-associated cytosolic protein and can also be found in the nucleus.

As used herein a “TREX1 inhibitor” is an agent that inhibits TREX1 enzymatic function (i.e., TREX1 exonuclease activity). In some embodiments, the TREX1 inhibitor is a small molecule such as TREX1 inhibitors described in US20200317677A1 and/or US20220017502A1, which are incorporated herein in their entireties. In some embodiments, the TREX1 inhibitor is an inhibitory oligonucleotide, such as a TREX1 siRNA or TREX1 antisense RNA.

### CRISPR Systems

As used herein, the phrase “CRISPR system” refers collectively to transcripts and other elements involved in the expression of or directing the activity of CRISPR-associated (“Cas”) genes, including sequences encoding a Cas gene, a tracr (trans-activating CRISPR) sequence (e.g. tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or other sequences and transcripts from a CRISPR locus. In embodiments of the disclosure the terms guide sequence and guide RNA are used interchangeably. In some embodiments, one or more elements of a CRISPR system is derived from a type I, type II, or type III CRISPR system. In some embodiments, one or more elements of a CRISPR system is derived from a particular organism comprising an endogenous CRISPR system, such as Streptococcus pyogenes. In general, a CRISPR system is characterized by elements that promote the formation of a CRISPR complex at the site of a target sequence (also referred to as a protospacer in the context of an endogenous CRISPR system). In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise any polynucleotide, such as DNA or RNA polynucleotides. In some embodiments, a target sequence is located in the nucleus or cytoplasm of a cell.

In some embodiments, direct repeats may be identified in silico by searching for repetitive motifs that fulfill any or all of the following criteria: 1. found in a 2 Kb window of genomic sequence flanking the type II CRISPR locus; 2. span from 20 to 50 bp; and 3. interspaced by 20 to 50 bp. In some embodiments, two of these criteria may be used, for instance 1 and 2, 2 and 3, or 1 and 3. In some embodiments, all three criteria may be used.

In some embodiments, candidate tracrRNA may be subsequently predicted by sequences that fulfill any or all of the following criteria: 1. sequence homology to direct repeats (motif search in Geneious with up to 18-bp mismatches); 2. presence of a predicted Rho-independent transcriptional terminator in direction of transcription; and 3. stable hairpin secondary structure between tracrRNA and direct repeat. In some embodiments, two of these criteria are used, for instance 1 and 2, 2 and 3, or 1 and 3. In some embodiments, all three criteria are used.

In some embodiments, chimeric synthetic guide RNAs (sgRNAs) designs may incorporate at least 12 bp of duplex structure between the direct repeat and tracrRNA.

In some embodiments, the CRISPR system is a type II CRISPR system and the Cas enzyme is Cas9, which catalyzes DNA cleavage. Enzymatic action by Cas9 derived from Streptococcus pyogenes or any closely related Cas9 generates double stranded breaks at target site sequences which hybridize to 20 nucleotides of the guide sequence and that have a protospacer-adjacent motif (PAM) sequence (examples include NGG/NRG or a PAM that can be determined as described herein) following the 20 nucleotides of the target sequence. CRISPR activity through Cas9 for site-specific DNA recognition and cleavage is defined by the guide sequence, the tracr sequence that hybridizes in part to the guide sequence and the PAM sequence. More aspects of the CRISPR system are described in Karginov and Hannon, The CRISPR system: small RNA-guided defense in bacteria and archaea, Mole Cell 2010 January 15; 37 (1): 7.

The type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). In this system, targeted DNA double-strand break (DSB) is generated in four sequential steps. First, two non-coding RNAs, the pre-crRNA array and tracrRNA, are transcribed from the CRISPR locus. Second, tracrRNA hybridizes to the direct repeats of pre-crRNA, which is then processed into mature crRNAs containing individual spacer sequences. Third, the mature crRNA: tracrRNA complex directs Cas9 to the DNA target consisting of the protospacer and the corresponding PAM via heteroduplex formation between the spacer region of the crRNA and the protospacer DNA. Finally, Cas9 mediates cleavage of target DNA upstream of PAM to create a DSB within the protospacer. To promote precise transcriptional initiation, the RNA polymerase III-based U6 promoter was selected to drive the expression of tracrRNA. Similarly, a U6 promoter-based construct was developed to express a pre-crRNA array consisting of a single spacer flanked by two direct repeats (DRs, also encompassed by the term “tracr-mate sequences”). The initial spacer was designed to target a 33-base-pair (bp) target site (30-bp protospacer plus a 3-bp CRISPR motif (PAM) sequence satisfying the NGG recognition motif of Cas9) in the human EMX1 locus, a key gene in the development of the cerebral cortex.

Typically, in the context of an endogenous CRISPR system, formation of a CRISPR complex (comprising a guide sequence hybridized to a target sequence and complexed with one or more Cas proteins) results in cleavage of one or both strands in or near (e.g., within 1, 2, 3, 4, 5, 6, 7, 8, 9, 10, 20, 50, or more base pairs from) the target sequence. Without wishing to be bound by theory, the tracr sequence, which may comprise or consist of all or a portion of a wild-type tracr sequence (e.g. about or more than about 20, 26, 32, 45, 48, 54, 63, 67, 85, or more nucleotides of a wild-type tracr sequence), may also form part of a CRISPR complex, such as by hybridization along at least a portion of the tracr sequence to all or a portion of a tracr mate sequence that is operably linked to the guide sequence. In some embodiments, one or more vectors driving expression of one or more elements of a CRISPR system are introduced into a host cell such that expression of the elements of the CRISPR system direct formation of a CRISPR complex at one or more target sites. For example, a Cas enzyme, a guide sequence linked to a tracr-mate sequence, and a tracr sequence could each be operably linked to separate regulatory elements on separate vectors. Alternatively, two or more of the elements expressed from the same or different regulatory elements, may be combined in a single vector, with one or more additional vectors providing any components of the CRISPR system not included in the first vector. CRISPR system elements that are combined in a single vector may be arranged in any suitable orientation, such as one element located 5′ with respect to (“upstream” of) or 3′ with respect to (“downstream” of) a second element. The coding sequence of one element may be located on the same or opposite strand of the coding sequence of a second element, and oriented in the same or opposite direction. In some embodiments, a single promoter drives expression of a transcript encoding a CRISPR enzyme and one or more of the guide sequence, tracr mate sequence (optionally operably linked to the guide sequence), and a tracr sequence embedded within one or more intron sequences (e.g. each in a different intron, two or more in at least one intron, or all in a single intron). In some embodiments, the CRISPR enzyme, guide sequence, tracr mate sequence, and tracr sequence are operably linked to and expressed from the same promoter.

In some embodiments, a vector comprises one or more insertion sites, such as a restriction endonuclease recognition sequence (also referred to as a “cloning site”). In some embodiments, one or more insertion sites (e.g., about or more than about 1, 2, 3, 4, 5, 6, 7, 8, 9, 10, or more insertion sites) are located upstream and/or downstream of one or more sequence elements of one or more vectors. In some embodiments, a vector comprises an insertion site upstream of a tracr mate sequence, and optionally downstream of a regulatory element operably linked to the tracr mate sequence, such that following insertion of a guide sequence into the insertion site and upon expression the guide sequence directs sequence-specific binding of a CRISPR complex to a target sequence in a eukaryotic cell. In some embodiments, a vector comprises two or more insertion sites, each insertion site being located between two tracr mate sequences so as to allow insertion of a guide sequence at each site. In such an arrangement, the two or more guide sequences may comprise two or more copies of a single guide sequence, two or more different guide sequences, or combinations of these. When multiple different guide sequences are used, a single expression construct may be used to target CRISPR activity to multiple different, corresponding target sequences within a cell. For example, a single vector may comprise about or more than about 1, 2, 3, 4, 5, 6, 7, 8, 9, 10, 15, 20, or more guide sequences. In some embodiments, about or more than about 1, 2, 3, 4, 5, 6, 7, 8, 9, 10, or more such guide-sequence-containing vectors may be provided, and optionally delivered to a cell.

In some embodiments, a vector comprises a regulatory element operably linked to an enzyme-coding sequence encoding a CRISPR enzyme, such as a Cas protein. Non-limiting examples of Cas proteins include Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologues thereof, or modified versions thereof. In some embodiments, the unmodified CRISPR enzyme has DNA cleavage activity, such as Cas9. In some embodiments, the Cas9 is an S. pyogenes Cas9 having the amino acid sequence:

(SEQ ID NO: 1)
MDKKYSIGLDIGTNSVGWAVITDDYKVPSKKFKVLGNTDRHSIKKNLIGA
LLFDSGETAEATRLKRTARRRYTRRKNRICYLQEIFSNEMAKVDDSFFHR
LEESFLVEEDKKHERHPIFGNIVDEVAYHEKYPTIYHLRKKLVDSTDKAD
LRLIYLALAHMIKFRGHFLIEGDLNPDNSDVDKLFIQLVQTYNQLFEENP
INASGVDAKAILSARLSKSRRLENLIAQLPGEKKNGLFGNLIALSLGLTP
NFKSNFDLAEDAKLQLSKDTYDDDLDNLLAQIGDQYADLFLAAKNLSDAI
LLSDILRVNTEITKAPLSASMIKRYDEHHQDLTLLKALVRQQLPEKYKEI
FFDQSKNGYAGYIDGGASQEEFYKFIKPILEKMDGTEELLVKLNREDLLR
KQRTFDNGSIPHQIHLGELHAILRRQEDFYPFLKDNREKIEKILTFRIPY
YVGPLARGNSRFAWMTRKSEETITPWNFEEVVDKGASAQSFIERMTNFDK
NLPNEKVLPKHSLLYEYFTVYNELTKVKYVTEGMRKPAFLSGEQKKAIVD
LLFKTNRKVTVKQLKEDYFKKIECFDSVEISGVEDRFNASLGTYHDLLKI
IKDKDFLDNEENEDILEDIVLTLTLFEDREMIEERLKTYAHLFDDKVMKQ
LKRRRYTGWGRLSRKLINGIRDKQSGKTILDFLKSDGFANRNFMQLIHDD
SLTFKEDIQKAQVSGQGDSLHEHIANLAGSPAIKKGILQTVKVVDELVKV
MGRHKPENIVIEMARENQTTQKGQKNSRERMKRIEEGIKELGSQILKEHP
VENTQLQNEKLYLYYLQNGRDMYVDQELDINRLSDYDVDHIVPQSFLKDD
SIDNKVLTRSDKNRGKSDNVPSEEVVKKMKNYWRQLLNAKLITQRKFDNL
TKAERGGLSELDKAGFIKRQLVETRQITKHVAQILDSRMNTKYDENDKLI
REVKVITLKSKLVSDFRKDFQFYKVREINNYHHAHDAYLNAVVGTALIKK
YPKLESEFVYGDYKVYDVRKMIAKSEQEIGKATAKYFFYSNIMNFFKTEI
TLANGEIRKRPLIETNGETGEIVWDKGRDFATVRKVLSMPQVNIVKKTEV
QTGGFSKESILPKRNSDKLIARKKDWDPKKYGGFDSPTVAYSVLVVAKVE
KGKSKKLKSVKELLGITIMERSSFEKNPIDFLEAKGYKEVKKDLIIKLPK
YSLFELENGRKRMLASAGELQKGNELALPSKYVNFLYLASHYEKLKGSPE
DNEQKQLFVEQHKHYLDEIIEQISEFSKRVILADANLDKVLSAYNKHRDK
PIREQAENIIHLFTLTNLGAPAAFKYFDTTIDRKRYTSTKEVLDATLIHQ
SITGLYETRIDLSQLGGD.

In some embodiments, the CRISPR enzyme directs cleavage of one or both strands at the location of a target sequence, such as within the target sequence and/or within the complement of the target sequence. In some embodiments, the CRISPR enzyme directs cleavage of one or both strands within about 1, 2, 3, 4, 5, 6, 7, 8, 9, 10, 15, 20, 25, 50, 100, 200, 500, or more base pairs from the first or last nucleotide of a target sequence. In some embodiments, a vector encodes a CRISPR enzyme that is mutated to with respect to a corresponding wild-type enzyme such that the mutated CRISPR enzyme lacks the ability to cleave one or both strands of a target polynucleotide containing a target sequence. In some embodiments, the residues to be mutated correspond to residues Asp10, His840, Asn854 and Asn863 in the wild type exemplary S. pyogenes Cas9 polypeptide (e.g., as determined by sequence and/or structural alignment). Non-limiting examples of mutations can include D10A, H840A, N854A or N863A. One skilled in the art will recognize that mutations other than alanine substitutions are suitable.

An aspartate-to-alanine substitution (D10A) in the RuvC I catalytic domain of SpCas9 was engineered to convert the nuclease into a nickase (SpCas9n) (see e.g., Sapranauskas et al., 2011, Nucleic Acis Research, 39:9275; Gasiunas et al., 2012, Proc. Natl. Acad. Sci. USA, 109: E2579), such that nicked genomic DNA undergoes the high-fidelity homology-directed repair (HDR). Surveyor assay confirmed that SpCas9n does not generate indels at the EMX1 protospacer target. Co-expression of EMX1-targeting chimeric crRNA (having the tracrRNA component as well) with SpCas9 produced indels in the target site, whereas co-expression with SpCas9n did not (n=3). Moreover, sequencing of 327 amplicons did not detect any indels induced by SpCas9n. The same locus was selected to test CRISPR-mediated HR by co-transfecting HEK 293 FT cells with the chimeric RNA targeting EMX1, hSpCas9 or hSpCas9n, as well as a HR template to introduce a pair of restriction sites (HindIII and NheI) near the protospacer.

Alternative orthologs are described herein. A Cas enzyme may be identified Cas9 as this can refer to the general class of enzymes that share homology to the biggest nuclease with multiple nuclease domains from the type II CRISPR system. In some embodiments, the Cas9 enzyme is from, or is derived from, spCas9 or saCas9. By derived, Applicants mean that the derived enzyme is largely based, in the sense of having a high degree of sequence homology with, a wildtype enzyme, but that it has been mutated (modified) in some way as described herein.

It will be appreciated that the terms Cas and CRISPR enzyme are generally used herein interchangeably, unless otherwise apparent. As mentioned above, many of the residue numberings used herein refer to the Cas9 enzyme from the type II CRISPR locus in Streptococcus pyogenes. However, it will be appreciated that this disclosure includes many more Cas9s from other species of microbes, such as SpCas9, SaCa9, StlCas9 and so forth.

In some embodiments, an enzyme coding sequence encoding a CRISPR enzyme is codon optimized for expression in particular cells, such as eukaryotic cells. The eukaryotic cells may be those of or derived from a particular organism, such as a mammal, including but not limited to human, mouse, rat, rabbit, dog, or non-human mammal or primate. In some embodiments, processes for modifying the germ line genetic identity of human beings and/or processes for modifying the genetic identity of animals which are likely to cause them suffering without any substantial medical benefit to man or animal, and also animals resulting from such processes, may be excluded.

In general, codon optimization refers to a process of modifying a nucleic acid sequence for enhanced expression in the host cells of interest by replacing at least one codon (e.g., about or more than about 1, 2, 3, 4, 5, 10, 15, 20, 25, 50, or more codons) of the native sequence with codons that are more frequently or most frequently used in the genes of that host cell while maintaining the native amino acid sequence. Various species exhibit particular bias for certain codons of a particular amino acid. Codon bias (differences in codon usage between organisms) often correlates with the efficiency of translation of messenger RNA (mRNA), which is in turn believed to be dependent on, among other things, the properties of the codons being translated and the availability of particular transfer RNA (tRNA) molecules. The predominance of selected tRNAs in a cell is generally a reflection of the codons used most frequently in peptide synthesis. Accordingly, genes can be tailored for optimal gene expression in a given organism based on codon optimization. Codon usage tables are readily available, for example, at the “Codon Usage Database” available at kazusa.orjp/codon/(visited Jul. 9, 2002), and these tables can be adapted in a number of ways. See Nakamura, Y., et al. “Codon usage tabulated from the international DNA sequence databases: status for the year 2000” Nucl. Acids Res. 28:292 (2000). Computer algorithms for codon optimizing a particular sequence for expression in a particular host cell are also available, such as Gene Forge (Aptagen; Jacobus, PA), are also available. In some embodiments, one or more codons (e.g., 1, 2, 3, 4, 5, 10, 15, 20, 25, 50, or more, or all codons) in a sequence encoding a CRISPR enzyme correspond to the most frequently used codon for a particular amino acid.

In some embodiments, a CRISPR enzyme comprises one or more nuclear localization sequences (NLSs), such as about or more than about 1, 2, 3, 4, 5, 6, 7, 8, 9, 10, or more NLSs. In some embodiments, a CRISPR enzyme comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g. one or more NLS at the amino-terminus and one or more NLS at the carboxy terminus). When more than one NLS is present, each may be selected independently of the others, such that a single NLS may be present in more than one copy and/or in combination with one or more other NLSs present in one or more copies. In a specific embodiment of the disclosure, the CRISPR enzyme comprises at most 6 NLSs. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. Non-limiting examples of NLSs include an NLS sequence derived from: the NLS of the SV40 virus large T-antigen, having the amino acid sequence PKKKRKV (SEQ ID NO: 2); the NLS from nucleoplasmin (e.g. the nucleoplasmin bipartite NLS with the sequence KRPAATKKAGQAKKKK (SEQ ID NO: 3); the c-myc NLS having the amino acid sequence PAAKRVKLD (SEQ ID NO: 4) or RQRRNELKRSP (SEQ ID NO: 5); the hRNPA1 M9 NLS having the sequence NQSSNFGPMKGGNFGGRSSGPYGGGGQYFAKPRNQGGY (SEQ ID NO: 6); the sequence RMRIZFKNKGKDTAELRRRRVEVSVELRKAKKDEQILKRRNV (SEQ ID NO: 7) of the IBB domain from importin-alpha; the sequences VSRKRPRP (SEQ ID NO: 8) and PPKKARED (SEQ ID NO: 9) of the myoma T protein; the sequence POPKKKPL (SEQ ID NO: 10) of human p53; the sequence SALIKKKKKMAP (SEQ ID NO: 11) of mouse c-abl IV; the sequences DRLRR (SEQ ID NO: 12) and PKQKKRK (SEQ ID NO: 13) of the influenza virus NS1; the sequence RKLKKKIKKL (SEQ ID NO: 14) of the Hepatitis virus delta antigen; the sequence REKKKFLKRR (SEQ ID NO: 15) of the mouse Mx1 protein; the sequence KRKGDEVDGVDEVAKKKSKK (SEQ ID NO: 16) of the human poly(ADP-ribose) polymerase; and the sequence RKCLQAGMNLEARKTKK (SEQ ID NO: 17) of the steroid hormone receptors (human) glucocorticoid.

In general, the one or more NLSs are of sufficient strength to drive accumulation of the CRISPR enzyme in a detectable amount in the nucleus of a eukaryotic cell. In general, strength of nuclear localization activity may derive from the number of NLSs in the CRISPR enzyme, the particular NLS(s) used, or a combination of these factors. Detection of accumulation in the nucleus may be performed by any suitable technique. For example, a detectable marker may be fused to the CRISPR enzyme, such that location within a cell may be visualized, such as in combination with a means for detecting the location of the nucleus (e.g. a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of CRISPR complex formation (e.g. assay for DNA cleavage or mutation at the target sequence, or assay for altered gene expression activity affected by CRISPR complex formation and/or CRISPR enzyme activity), as compared to a control no exposed to the CRISPR enzyme or complex, or exposed to a CRISPR enzyme lacking the one or more NLSs.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAT, Novoalign (Novocraft Technologies; available at www.novocraft.com), ELAND (Illumina, San Diego, CA), SOAP (available at soap.genomics.org.cn), and Maq (available at maq.sourceforge.net). In some embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In some embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

A guide sequence may be selected to target any target sequence. In some embodiments, the target sequence is a sequence within a genome of a cell. Exemplary target sequences include those that are unique in the target genome. For example, for the S. pyogenes Cas9, a unique target sequence in a genome may include a Cas9 target site NNNNNNNNNNNNXGG (SEQ ID NO: 18) (N is A, G, T, or C; and X can be A, G, T, or C or an analog thereof) has a single occurrence in the genome. A unique target sequence in a genome may include an S. pyogenes Cas9 target site NNNNNNNNNNNXGG (SEQ ID NO: 19) (N is A, G, T, or C; and X can be A, G, T, or C) has a single occurrence in the genome. For the S. thermophilus CRISPR1 Cas9, a unique target sequence in a genome may include a Cas9 target site NNNNNNNNNNNNXXAGAAW (SEQ ID NO: 20) (N is A, G, T, or C; X can be A, G, T, or C; and W is A or T) has a single occurrence in the genome. A unique target sequence in a genome may include an S. thermophilus CRISPR1 Cas9 target site NNNNNNNNNNNXXAGAAW (SEQ ID NO: 21) (N is A, G, T, or C; X can be A, G, T, or C; and W is A or T) has a single occurrence in the genome. For the S. pyogenes Cas9, a unique target sequence in a genome may include a Cas9 target site NNNNNNNNNNNNXGGXG (SEQ ID NO: 22) (N is A, G, T, or C; and X can be A, G, T, or C) has a single occurrence in the genome. A unique target sequence in a genome may include an S. pyogenes Cas9 target site NNNNNNNNNNNXGGXG (SEQ ID NO: 23) (N is A, G, T, or C; and X can be A, G, T, or C) has a single occurrence in the genome.

In some embodiments, a guide sequence is selected to reduce the degree secondary structure within the guide sequence. In some embodiments, about or less than about 75%, 50%, 40%, 30%, 25%, 20%, 15%, 10%, 5%, 1%, or fewer of the nucleotides of the guide sequence participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g. A. R. Gruber et al., 2008, Cell 106 (1): 23-24; and PA Carr and GM Church, 2009, Nature Biotechnology 27 (12): 1151-62).

In general, a tracr mate sequence includes any sequence that has sufficient complementarity with a tracr sequence to promote one or more of: (1) excision of a guide sequence flanked by tracr mate sequences in a cell containing the corresponding tracr sequence; and (2) formation of a CRISPR complex at a target sequence, wherein the CRISPR complex comprises the tracr mate sequence hybridized to the tracr sequence. In general, degree of complementarity is with reference to the optimal alignment of the tracr mate sequence and tracr sequence, along the length of the shorter of the two sequences. Optimal alignment may be determined by any suitable alignment algorithm, and may further account for secondary structures, such as self-complementarity within either the tracr sequence or tracr mate sequence. In some embodiments, the degree of complementarity between the tracr sequence and tracr mate sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and tracr mate sequence are contained within a single transcript, such that hybridization between the two produces a transcript having a secondary structure, such as a hairpin. In an embodiment of the disclosure, the transcript or transcribed polynucleotide sequence has at least two or more hairpins. In some embodiments, the transcript has two, three, four or five hairpins. In a further embodiment of the disclosure, the transcript has at most five hairpins. In a hairpin structure the portion of the sequence 5′ of the final “N” and upstream of the loop corresponds to the tracr mate sequence, and the portion of the sequence 3′ of the loop corresponds to the tracr sequence Further non-limiting examples of single polynucleotides comprising a guide sequence, a tracr mate sequence, and a tracr sequence are as follows (listed 5′ to 3′), where “N” represents a base of a guide sequence (N is A, G, T, or C), the first block of lower case letters represent the tracr mate sequence, and the second block of lower case letters represent the tracr sequence, and (1) the final poly-T sequence represents the transcription terminator: NNNNNNNNNNNNNNNNNNNNgtttttgtactctcaagatttaGAAAtaaatcttgcagaagctacaaagataag gcttcatgccgaaatcaacaccctgtcattttatggcagggtgttttcgttatttaaTTTTTT (SEQ ID NO:24); (2) NNNNNNNNNNNNNNNNNNNNgtttttgtactctcaGAAAtgcagaagctacaaagataaggcttcatgccgaa atcaacaccctgtcattttatggcagggtgttttcgttatttaaTTTTTT (SEQ ID NO: 25); (3) NNNNNNNNNNNNNNNNNNNNgtttttgtactctcaGAAAtgcagaagctacaaagataaggcttcatgccgaa atcaacaccctgtcattttatggcagggtgtTTTTTT (SEQ ID NO: 26); (4) NNNNNNNNNNNNNNNNNNNNgttttagagctaGAAAtagcaagttaaaataaggctagtccgttatcaacttga aaaagtggcaccgagtcggtgcTTTTTT (SEQ ID NO: 27); (5) NNNNNNNNNNNNNNNNNNNNgttttagagctaGAAATAGcaagttaaaataaggctagtccgttatcaactt gaaaaagtgTTTTTTT (SEQ ID NO: 28); and (6) NNNNNNNNNNNNNNNNNNNNgttttagagctagAAATAGcaagttaaaataaggctagtccgttatcaTT TTTTTT (SEQ ID NO: 29). In some embodiments, sequences (1) to (3) are used in combination with Cas9 from S. thermophilus CRISPR1. In some embodiments, sequences (4) to (6) are used in combination with Cas9 from S. pyogenes. In some embodiments, the tracr sequence is a separate transcript from a transcript comprising the tracr mate sequence.

In some embodiments, the CRISPR enzyme is part of a fusion protein comprising one or more heterologous protein domains (e.g., about or more than about 1, 2, 3, 4, 5, 6, 7, 8, 9, 10, or more domains in addition to the CRISPR enzyme). A CRISPR enzyme fusion protein may comprise any additional protein sequence, and optionally a linker sequence between any two domains. Examples of protein domains that may be fused to a CRISPR enzyme include, without limitation, epitope tags, reporter gene sequences, and protein domains having one or more of the following activities: methylase activity, demethylase activity, transcription activation activity, transcription repression activity, transcription release factor activity, histone modification activity, RNA cleavage activity and nucleic acid binding activity. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporter genes include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and autofluorescent proteins including blue fluorescent protein (BFP). A CRISPR enzyme may be fused to a gene sequence encoding a protein or a fragment of a protein that bind DNA molecules or bind other cellular molecules, including but not limited to maltose binding protein (MBP), S-tag, Lex A DNA binding domain (DBD) fusions, GAL4 DNA binding domain fusions, and herpes simplex virus (HSV) BP16 protein fusions. Additional domains that may form part of a fusion protein comprising a CRISPR enzyme are described in US20110059502, incorporated herein by reference. In some embodiments, a tagged CRISPR enzyme is used to identify the location of a target sequence.

In some embodiments, a CRISPR enzyme may form a component of an inducible system. The inducible nature of the system would allow for spatiotemporal control of gene editing or gene expression using a form of energy. The form of energy may include but is not limited to electromagnetic radiation, sound energy, chemical energy and thermal energy. Examples of inducible system include tetracycline inducible promoters (Tet-On or Tet-Off), small molecule two-hybrid transcription activations systems (FKBP, ABA, etc), or light inducible systems (Phytochrome, LOV domains, or cryptochrome). In one embodiment, the CRISPR enzyme may be a part of a Light Inducible Transcriptional Effector (LITE) to direct changes in transcriptional activity in a sequence-specific manner. The components of a light may include a CRISPR enzyme, a light-responsive cytochrome heterodimer (e.g., from Arabidopsis thaliana), and a transcriptional activation/repression domain. Further examples of inducible DNA binding proteins and methods for their use are provided in U.S. 61/736,465 and U.S. 61/721,283, which is hereby incorporated by reference in its entirety.

In some aspects, the disclosure provides methods comprising delivering one or more polynucleotides, such as or one or more vectors as described herein, one or more transcripts thereof, and/or one or proteins transcribed therefrom, to a host cell. In some aspects, the disclosure further provides cells produced by such methods, and animals comprising or produced from such cells. In some embodiments, a CRISPR enzyme in combination with (and optionally complexed with) a guide sequence is delivered to a cell. Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256:808-813 (1992); Nabel & Felgner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6 (10): 1149-1154 (1988); Vigne, Restorative Neurology and Neuroscience 8:35-36 (1995); Kremer & Perricaudet, British Medical Bulletin 51 (1): 31-44 (1995); Haddada et al., in Current Topics in Microbiology and Immunology Doerfler and Böhm (eds) (1995); and Yu et al., Gene Therapy 1:13-26 (1994).

Methods of non-viral delivery of nucleic acids include lipofection, microinjection, biolistics, virosomes, liposomes (e.g., lipid nanoparticles), immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Felgner, WO 91/17424; WO 91/16024. Delivery can be to cells (e.g. in vitro or ex vivo administration) or target tissues (e.g. in vivo administration).

The preparation of lipid: nucleic acid complexes, including targeted liposomes such as immunolipid complexes, is well known to one of skill in the art (see, e.g., Crystal, Science 270:404-410 (1995); Blaese et al., Cancer Gene Ther. 2:291-297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLa-S3, Huh1, Huh4, Huh7, HUVEC, HASMC, HEKn, HEKa, MiaPaCell, Panc1, PC-3, TF1, CTLL-2, CIR, Rat6, CV1, RPTE, A10, T24, J82, A375, ARH-77, Calu1, SW480, SW620, SKOV3, SK-UT, CaCo2, P388D1, SEM-K2, WEHI-231, HB56, TIB55, Jurkat, J45.01, LRMB, Bcl-1, BC-3, IC21, DLD2, Raw264.7, NRK, NRK-52E, MRC5, MEF, Hep G2, HeLa B, HeLa T4, COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-1 cells, BEAS-2B, bEnd.3, BHK-21, BR 293, BxPC3, C3H-10T1/2, C6/36, Cal-27, CHO, CHO-7, CHO-IR, CHO-K1, CHO-K2, CHO-T, CHO Dhfr−/−, COR-L23, COR-L23/CPR, COR-L23/5010, COR-L23/R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepa1c1c7, HL-60, HMEC, HT-29, Jurkat, JY cells, K562 cells, Ku812, KCL22, KG1, KYO1, LNCap, Ma-Mel 1-48, MC-38, MCF-7, MCF-10A, MDA-MB-231, MDA-MB-468, MDA-MB-435, MDCK II, MDCK II, MOR/0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/PNT 2, RenCa, RIN-5F, RMA/RMAS, Saos-2 cells, Sf-9, SkBr3, T2, T-47D, T84, THP 1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)). In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds. In one aspect, the cell or cell line is a stem cell.

### TALEN Systems

As used herein, the term “TALEN” refers to a nucleic acid encoding a protein comprising a TALE DNA binding domain fused to an endonuclease, resulting in a monomeric TALEN. A monomeric TALEN can be functional. A monomeric TALEN can be capable of dimerizing with another monomeric TALEN. Such dimerization can be a result of a homodimeric TALEN wherein both monomeric TALENs are the same (e.g., identical). Alternatively, the dimerization can be a result of the dimerization of two monomeric TALENs that are different. For example, two monomeric TALENs can be different when the repeat variable diresidues of each monomeric TALEN are different.

As used herein, the term “TALEN” refers to a nucleic acid encoding a protein comprising a TALE DNA binding domain fused to an endonuclease, resulting in a monomeric TALEN. A monomeric TALEN can be functional. A monomeric TALEN can be capable of dimerizing with another monomeric TALEN. Such dimerization can be a result of a homodimeric TALEN wherein both monomeric TALENs are the same (e.g., identical). Alternatively, the dimerization can be a result of the dimerization of two monomeric TALENs that are different. For example, two monomeric TALENs can be different when the repeat variable diresidues of each monomeric TALEN are different.

In an aspect, the TALEN can be optimized for expression in a human cell. For example, the nucleic acid sequence can be codon optimized for efficient expression (e.g., transcription and/or translation) in mammalian cells, i.e., “humanized.” Methods of codon optimization are well-known in the art. Alternatively, the TALEN sequence can be inserted into a commercially available, for example, a vector such as JDS70, JDS71, JDS74 and JDS78 from Addgene (Cambridge, Mass.). The TALE nucleotide sequence can be modified to encode biologically active variants of a TALEN, and these variants can have or include, for example, an amino acid sequence that differs from a wild type TALEN by containing one or more mutations (e.g., an addition, deletion, or substitution mutation or a combination of such mutations). One or more substitution mutations can be a substitution (e.g., a conservative amino acid substitution). For example, a biologically active variant of a TALE polypeptide can have an amino acid sequence with at least or about 50% sequence identity (e.g., at least or about 50%, 55%, 60%, 65%, 70%, 75%, 80%, 85%, 90%, 95%, 97%, 98%, or 99% sequence identity) to a wild type TALE polypeptide. Conservative amino acid substitutions typically include substitutions within the following groups: glycine and alanine; valine, isoleucine, and leucine; aspartic acid and glutamic acid; asparagine, glutamine, serine and threonine; lysine, histidine and arginine; and phenylalanine and tyrosine. The amino acid residues in the TALE amino acid sequence can be non-naturally occurring amino acid residues. Naturally occurring amino acid residues include those naturally encoded by the genetic code as well as non-standard amino acids (e.g., amino acids having the D-configuration instead of the L-configuration). The present peptides can also include amino acid residues that are modified versions of standard residues (e.g., pyrrolysine can be used in place of lysine and selenocysteine can be used in place of cysteine). Non-naturally occurring amino acid residues are those that have not been found in nature, but that conform to the basic formula of an amino acid and can be incorporated into a peptide. These include D-alloisoleucine (2R,3S)-2-amino-3-methylpentanoic acid and L-cyclopentyl glycine(S)-2-amino-2-cyclopentyl acetic acid.

The TALENs described herein can include a C-terminal truncated TAL repeat in the TALE DNA binding domain. The C-terminal truncated TAL repeat can be about 34 or few amino acids. For example, the C-terminal truncated TAL repeat can be 33, 32, 31, 30, 29, and so on amino acids in length or less. The C-terminal truncated TAL repeat regardless of length still contains the RVDs for specificity for a nucleotide. In an aspect, the TALE DNA binding domain comprises a C-terminal truncated TAL repeat.

In an aspect, the TALEN can further comprise a nuclear localization signal (NLS) for import into a cell nucleus. NLS sequences are short segments of amino acids comprising positively charged lysines or arginines or a combination thereof. Generally, a NLS contains positively charged residues since it is well-known that positive residues bind importins to gain access into the nucleus of a cell. The NLS can be any sequence from a nuclear encoded protein. Non-limiting examples of NLSs include an NLS sequence derived from: the NLS of the SV40 virus large T-antigen, having the amino acid sequence PKKKRKV (SEQ ID NO: 2); the NLS from nucleoplasmin (e.g. the nucleoplasmin bipartite NLS with the sequence KRPAATKKAGQAKKKK (SEQ ID NO: 3); the c-myc NLS having the amino acid sequence PAAKRVKLD (SEQ ID NO: 4) or RQRRNELKRSP (SEQ ID NO: 5); the hRNPA1 M9 NLS having the sequence NQSSNFGPMKGGNFGGRSSGPYGGGGQYFAKPRNQGGY (SEQ ID NO: 6); the sequence RMRIZFKNKGKDTAELRRRRVEVSVELRKAKKDEQILKRRNV (SEQ ID NO: 7) of the IBB domain from importin-alpha; the sequences VSRKRPRP (SEQ ID NO: 8) and PPKKARED (SEQ ID NO: 9) of the myoma T protein; the sequence POPKKKPL (SEQ ID NO: 10) of human p53; the sequence SALIKKKKKMAP (SEQ ID NO: 11) of mouse c-abl IV; the sequences DRLRR (SEQ ID NO: 12) and PKQKKRK (SEQ ID NO: 13) of the influenza virus NS1; the sequence RKLKKKIKKL (SEQ ID NO: 14) of the Hepatitis virus delta antigen; the sequence REKKKFLKRR (SEQ ID NO: 15) of the mouse Mx1 protein; the sequence KRKGDEVDGVDEVAKKKSKK (SEQ ID NO: 16) of the human poly(ADP-ribose) polymerase; and the sequence RKCLQAGMNLEARKTKK (SEQ ID NO: 17) of the steroid hormone receptors (human) glucocorticoid. The NLS can be positioned at the N-terminal or C-terminal end of the TALEN described herein. In an aspect, the NLS is positioned at the N-terminus.

Spacers. The TALENs described herein can comprise spacer sequences. Spacer sequences are oriented on either side of the TALE DNA binding domain and are sufficiently long enough to permit two FokI catalytic domains to dimerize and cleave the DNA. In other words, the spacer length should be sufficiently long enough to separate the TALE binding sites to enable the two FokI catalytic domains to efficiently dimerize. The spacer sequence can also be selected or varied to modulate TALEN specificity and activity. As disclosed herein, a TALEN comprises from the N-terminus to the C-terminus, a first spacer sequence, a TALE DNA binding domain, a second spacer sequence, and a FokI nuclease catalytic domain fused to the C-terminus. The spacer sequences can be about 10 to about 200 amino acids in length.

In an aspect, the first spacer sequence is fused to a nuclear localization signal (NLS) as described herein. The first spacer sequence can also be fused to one or more labels or detection tags. Further, the first spacer sequence can comprise an N-terminal portion of the coding region for a TALE, for example, the first space sequence can be derived from N-terminal portion of a Xanthomonas TALE. In an aspect, the second spacer sequence comprises part of a C-terminal TALE domain. In some aspects, the second spacer comprises a truncated C-terminal TALE domain. The second spacer can also be fused to the NLS and/or to one or more labels or detection tags.

In some embodiments, TALENs can also include a nuclease catalytic domain selected from a FokI, AarI, Acelll, AcH, Alol, Bael, Bbrll, CdH, QePl, Edl, Esp31, Finl, Mbol, sapl, or SspD51 nuclease catalytic domain. In some embodiments, a TALEN comprises a FokI nuclease catalytic domain. In some embodiments, a FokI nuclease catalytic domain is encoded by SEQ ID NO: 30 or SEQ ID NO: 31. In some embodiments, a FokI nuclease catalytic domain comprises SEQ ID NO: 32 or SEQ ID NO: 33. In an aspect, the FokI nuclease catalytic domain is an obligate homodimer or an obligate heterodimer. The FokI nuclease catalytic domain dimerizes to form an active nuclease, thus two TALENs (e.g., two TALEN monomers) or a pair of TALENs makes a functional genome-editing endonuclease. For example, for a pair of TALENs, each contain one member of the obligate heterodimer pair binds to the adjacent (e.g., within 10, 11, 12, 13, 14, 15, 16, 17, 18, 19, 20, 21, 22, 23, 24, 25 or more continuous nucleotides) recognition site (i.e., effector binding element) to enable dimerization of the endonuclease for cleavage. In an aspect, each effector binding element of an obligate heterodimer pair can independently be about 10, 11, 12, 13, 14, 15, 16, 17, 18, 19, 20, 21, 22, 23, 24, 25, 26, 27, 28, 29, 30 or more base pairs in length. Each monomer of an obligate dimer pair recognizes one strand of a target double-stranded nucleic acid and the other monomer of the obligate dimer pair recognizes the other strand of a target double-stranded nucleic acid. The effector binding elements are generally sufficiently separated by the first and second spacer components where the DNA cleavage occurs.

In some aspects, the catalytic domain can be a nuclease catalytic domain for a nuclease other than FokI.

Polynucleotides. In an aspect, polynucleotides or polynucleotide sequences encoding the engineered TALENs are disclosed. The polynucleotides or polynucleotide sequences can be optimized for expression in a subject (e.g., human). Methods of codon optimization are well-known in the art.

In an aspect, the present disclosure provides mRNAs encoding any of the TALENs described herein. For example, the mRNA can be obtained via in vitro or in vivo transcription. Such mRNA can be used for the translation into TALENs in a host cell. Examples of administering the mRNA to a host cell include injection, or transfection using polyethylenimine, lipid, or calcium phosphate, or electroporation. The mRNA can also be fused to a translocation domain that is useful for translocation across a cell membrane. Once the mRNA is present inside the host cell, TALEN synthesis can begin.

In some embodiments, the TALENs of the instant disclosure include the TALENs described in U.S. Pat. No. 11,046,959, which is incorporated herein in its entirety.

### Zinc Finger Nuclease (ZFN) Systems

The zinc finger domains of Zinc finger nucleases (ZFNs) consist of 2 or more tandemly arrayed zinc finger modules, each of which recognizes 3 bp sub-sites. Since each module independently recognizes DNA sequences, the zinc finger domains consisting of 2 to 4 modules are able to bind to a 6- or 12-bp sequence. Zinc finger nucleases function as dimmers and, therefore, a pair of zinc finger nuclease consisting of 2 to 4 zinc finger modules specifically recognizes 12- to 24-bp sequence. In a specific example, the zinc finger nucleases of the present disclosure have the zinc finger domains consisting of 2 or more, e.g., 2 to 4, or 3 zinc finger modules.

In some embodiments, ZFNs are modular proteins comprised of an engineered zinc finger DNA binding domain linked to the catalytic domain of an endonuclease. In some embodiments, the ZFNs can also include a nuclease catalytic domain selected from a FokI, Aarl, Acelll, AcH, Alol, Bael, Bbrll, CdH, QePl, Edl, Esp3l, Finl, Mbol, sapl, or SspD51 nuclease catalytic domain.

In some embodiments, a TALEN comprises a FokI nuclease catalytic domain. In some embodiments, a FokI nuclease catalytic domain is encoded by SEQ ID NO: 30 or SEQ ID NO: 31. In some embodiments, a FokI nuclease catalytic domain comprises SEQ ID NO: 32 or SEQ ID NO: 33. In an aspect, the FokI nuclease catalytic domain is an obligate homodimer or an obligate heterodimer.

Since endonuclease catalytic domains of the instant disclosure function only as dimers, a pair of ZFNs are engineered to bind to cognate target “half-site” sequences on opposite DNA strands and with precise spacing between them to enable the catalytically active endonuclease catalytic domain dimer to form. Upon dimerization of the endonuclease catalytic domain, which itself has no sequence specificity per se, a DNA double-strand break is generated between the ZFN half-sites as the initiating step in genome editing.

The DNA binding domain of each ZFN is typically comprised of 3-6 zinc fingers of the abundant Cys2-His2 architecture, with each finger primarily recognizing a triplet of nucleotides on one strand of the target DNA sequence, although cross-strand interaction with a fourth nucleotide also can be important. Alteration of the amino acids of a finger in positions that make key contacts with the DNA alters the sequence specificity of a given finger. Thus, a four-finger zinc finger protein will selectively recognize a 12 bp target sequence, where the target sequence is a composite of the triplet preferences contributed by each finger, although triplet preference can be influenced to varying degrees by neighboring fingers. An important aspect then of ZFNs is that they can be readily retargeted to almost any genomic address simply by modifying individual fingers, although considerable expertise is required to do this well. In most applications of ZFNs, proteins of 4-6 fingers are used, recognizing 12-18 bp respectively. Hence, a pair of ZFNs will typically recognize a combined target sequence of 24-36 bp, not including the 5-7 bp spacer between half-sites. A target sequence of this length is likely to be unique in the human genome, assuming repetitive sequences or gene homologs are excluded during the design process. Nevertheless, the ZFN protein-DNA interactions are not absolute in their specificity and so off-target binding and cleavage events do occur, either as a heterodimer between the two ZFNs, or as a homodimer of one or other of the ZFNs. The latter possibility has been effectively eliminated by engineering the dimerization interface of the Fokl domain to create “plus” and “minus” variants, also known as obligate heterodimer variants, which can only dimerize with each other and not with themselves. Forcing the obligate heterodimer prevents formation of the homodimer. This has greatly enhanced specificity of ZFNs as well as of any other nuclease that adopts these Fokl variants.

A variety of ZFN-based systems have been described in the art, modifications thereof are regularly reported, and numerous references describe rules and parameters that are used to guide the design of ZFNs; see, e.g., Segal et al., Proc Natl Acad Sci USA 96 (6): 2758-63 (1999); Dreier B et al., J Mol Biol. 303 (4): 489-502 (2000); Liu Q et al., J Biol Chem. 277 (6): 3850-6 (2002); Dreier et al., J Biol Chem 280 (42): 35588-97 (2005); and Dreier et al., J Biol Chem. 276 (31): 29466-78 (2001).

Target Polynucleotide (“Target sequence”)

The target polynucleotide of a gene-editing system can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA).

Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease. The transcribed or translated products may be known or unknown, and may be at a normal or abnormal level.

The target polynucleotide of a gene-editing system can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA).

The target polynucleotide of a gene-editing system may include a number of disease-associated genes and polynucleotides as well as signaling biochemical pathway-associated genes and polynucleotides as listed in U.S. provisional patent applications 61/736,527 and 61/748,427 having Broad reference BI-2011/008/WSGR Docket No. 44063-701.101 and BI-2011/008/WSGR Docket No. 44063-701.102 respectively, both entitled SYSTEMS METHODS AND COMPOSITIONS FOR SEQUENCE MANIPULATION filed on Dec. 12, 2012 and Jan. 2, 2013, respectively, the contents of all of which are herein incorporated by reference in their entirety.

Further examples of genes, diseases and proteins are hereby incorporated by reference from WO 2014/093595 and WO 2014/093622. Such genes, proteins and pathways may be the target polynucleotide of a gene-editing system.

In some embodiments, the instant disclosure includes the ZFNs described in U.S. Pat. No. 10,738,305B2, which is incorporated herein in its entirety.

In some embodiments, the target sequence comprises one or more of CF Transmembrane Conductance Regulator (“CFTR”, NCBI Entrez Gene: 1080 Ensembl: ENSG00000001626 OMIM®: 602421 UniProtKB/Swiss-Prot: P13569), C-C Motif Chemokine Receptor 5 (CCR5, HGNC: 1606 NCBI Entrez Gene: 1234 Ensembl: ENSG00000160791 OMIM®: 601373 UniProtKB/Swiss-Prot: P51681) and Hemoglobin Subunit Beta (“HBB”, HGNC: 4827 NCBI Entrez Gene: 3043 Ensembl: ENSG00000244734 OMIM®: 141900 UniProtKB/Swiss-Prot: P68871) loci.

### Homology-Directed Repair (HDR) and HDR Template

In one embodiment, this disclosure provides a method of cleaving a target polynucleotide using a gene-editing system. The method comprises modifying a target polynucleotide using a gene editing system that binds to the target polynucleotide and effect cleavage of said target polynucleotide. Typically, the gene editing system, when introduced into a cell, creates a break (e.g., a single or a double strand break) in the genome sequence. For example, the method can be used to cleave a disease gene in a cell.

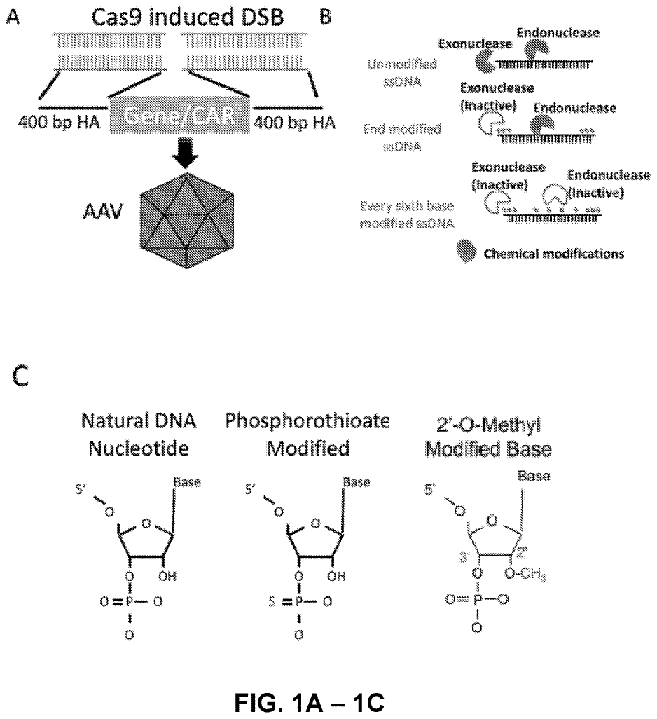

The break created by the gene editing system can be repaired by a repair process such as the error prone non-homologous end joining (NHEJ) pathway or the high fidelity homology-directed repair (HDR) (FIG. 1A). During these repair process, an exogenous polynucleotide template (an “HDR template polynucleotide” or “HR template”) can be introduced into the genome sequence. In some methods, the HDR process is used modify genome sequence. For example, an HDR template polynucleotide comprising, or consisting essentially of, or consisting of a sequence to be integrated flanked by an upstream sequence and a downstream sequence is introduced into a cell. The upstream and downstream sequences share sequence similarity with either side of the site of integration in the chromosome.

The HDR template polynucleotide comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

The upstream and downstream sequences in the HDR template polynucleotide are selected to promote recombination between the chromosomal sequence of interest and the donor polynucleotide. The upstream sequence is a nucleic acid sequence that shares sequence similarity with the genome sequence upstream of the targeted site for integration. Similarly, the downstream sequence is a nucleic acid sequence that shares sequence similarity with the chromosomal sequence downstream of the targeted site of integration. The upstream and downstream sequences in the HDR template polynucleotide can have 75%, 80%, 85%, 90%, 95%, or 100% sequence identity with the targeted genome sequence. In some embodiments, the upstream and downstream sequences in HDR template polynucleotide have about 95%, 96%, 97%, 98%, 99%, or 100% sequence identity with the targeted genome sequence. In some methods, the upstream and downstream sequences in the HDR template polynucleotide have about 99% or 100% sequence identity with the targeted genome sequence.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000 bp.

In some methods, the HDR template polynucleotide may further comprise a marker. Such a marker may make it easy to screen for targeted integrations. Examples of suitable markers include restriction sites, fluorescent proteins, or selectable markers. The HDR template polynucleotide of the disclosure can be constructed using recombinant techniques (see, for example, Sambrook et al., Molecular Cloning: a Laboratory Manual. Cold Spring Harbor Laboratory press, 2001).

In an exemplary method for modifying a target polynucleotide by integrating an HDR template polynucleotide, a double stranded break is introduced into the genome sequence by the CRISPR complex, the break is repaired via homologous recombination an HDR template polynucleotide such that the template is integrated into the genome. The presence of a double-stranded break facilitates integration of the template.

In some embodiments, AZD7648 (as described in Goldberg, Frederick W., et al. “The Discovery of 7-Methyl-2-[(7-methyl[1, 2, 4]triazolo[1,5-a]pyridin-6-yl)amino]-9-(tetrahydro-2H-pyran-4-yl)-7, 9-dihydro-8H-purin-8-one (AZD7648), a Potent and Selective DNA-Dependent Protein Kinase (DNA-PK) Inhibitor.” (2019): 3461-3471, which is incorporated herein in its entirety; BOC Sciences Catalog Number: 2230820-11-6) has the following chemical formula:

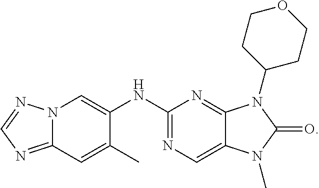 [SMILES: Cc1cc2ncnn2cc1Nc1ncc2c(n1)n(C1CCOCC1)c(=O)n2C]

In some embodiments, M3814 (peposertib or Nedisertib) (as described in Zenke, Frank T., et al. “Pharmacologic inhibitor of DNA-PK, M3814, potentiates radiotherapy and regresses human tumors in mouse models.” Molecular cancer therapeutics 19.5 (2020): 1091-1101, which is incorporated herein in its entirety) has the following chemical formula:

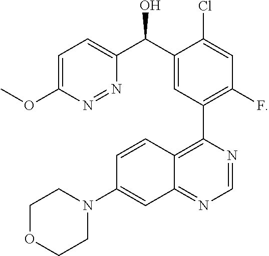 [SMILES: COc1ccc([C@@H](O)c2cc(-c3ncnc4cc(N5CCOCC5)ccc34)c(F)cc2Cl)nn1]

In some embodiments, M9831 (VX-984) (as described in Khan, Atif J., et al. “VX-984 is a selective inhibitor of non-homologous end joining, with possible preferential activity in transformed cells.” Oncotarget 9.40 (2018): 25833, which is incorporated herein in its entirety) has the following chemical structure:

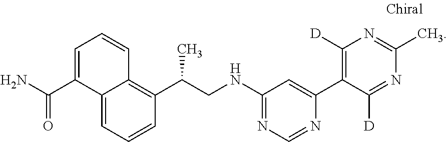 [SMILES: [2H]c1nc(C)nc([2H])c1-c1cc(NC[C@@H](C)c2cccc3c(C(N)=O)cccc23)ncn1]

In some embodiments, BAY-8400 (as described in Berger, Markus, et al. “BAY-8400: a novel potent and selective DNA-PK inhibitor which shows synergistic efficacy in combination with targeted alpha therapies.” Journal of Medicinal Chemistry 64.17 (2021): 12723-12737, which is incorporated herein in its entirety) has the following chemical structure:

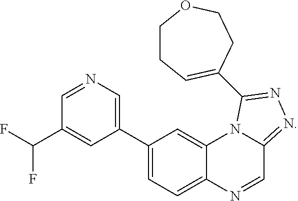 [SMILES: FC(F)c1cncc(-c2ccc3ncc4nnc(C5=CCCOCC5)n4c3c2)c1]

### Modes for Carrying Out the Disclosure

Previous studies investigated the modification of the ends of ssDNA HDR templates to improve their stability with mixed results. Applicant has found that extensive chemical modification of an ssDNA HDR template significantly and unexpectedly improves the editing efficiency of ssDNA-based HDR templates. Without being bound to a particular theory, it is believed that extensive chemical modification of ssDNA prevents degradation of the ssDNA by cellular endo and exonucleases and improves gene editing.

### Gene-Editing Systems

An aspect of the disclosure is directed to a gene-editing system comprising, or consisting essentially of, or consisting of: components of a gene editing system selected from a CRISPR-Cas system, a TALEN system and a Zinc Finger Nuclease (ZFN) system; and a single stranded homology directed repair (HDR) template polynucleotide (also known as an “HR template”), wherein the first three nucleotides at the 5′ end and the last three nucleotides at the 3′ end of the HDR repair template polynucleotide comprise a chemically modified nucleotide; and wherein the nucleotides between the 5′end and the 3′ end of the HDR repair template polynucleotide comprises a chemically modified nucleotide every 4, 5, 6, 7 or 8 nucleotides along at least 95%, at least 96%, at least 97%, at least 98%, at least 99% or more of the length of the HDR template polynucleotide. In some embodiments, the least 95%, at least 96%, at least 97%, at least 98%, at least 99% or more of the length of the HDR template polynucleotide comprises the entire length of the HDR template polynucleotide, including the first three nucleotides at the 5′ end and the last three nucleotides at the 3′ end of the HDR repair template polynucleotide. In some embodiments, the least 95%, at least 96%, at least 97%, at least 98%, at least 99% or more of the length of the HDR template polynucleotide excludes the first three nucleotides at the 5′ end and the last three nucleotides at the 3′ end of the HDR repair template polynucleotide. In one aspect, one or more of the components of the system are detectably labeled.

An aspect of the disclosure is directed to a gene-editing system comprising, or consisting essentially of, or consisting of: components of a gene editing system selected from a CRISPR-Cas system, a TALEN system and a Zinc Finger Nuclease (ZFN) system; and a single stranded homology directed repair (HDR) template polynucleotide, wherein the first three nucleotides at the 5′ end and the last three nucleotides at the 3′ end of the HDR repair template polynucleotide comprise a chemically modified nucleotide; and wherein the nucleotides between the 5′end and the 3′ end of the HDR repair template polynucleotide comprises a chemically modified nucleotide every 4, 5, 6, 7, 8, 9 or 10 nucleotides along at least 95%, at least 96%, at least 97%, at least 98%, at least 99% or more of the length of the HDR template polynucleotide. In some embodiments, the least 95%, at least 96%, at least 97%, at least 98%, at least 99% or more of the length of the HDR template polynucleotide comprises the entire length of the HDR template polynucleotide, including the first three nucleotides at the 5′ end and the last three nucleotides at the 3′ end of the HDR repair template polynucleotide. In some embodiments, the least 95%, at least 96%, at least 97%, at least 98%, at least 99% or more of the length of the HDR template polynucleotide excludes the first three nucleotides at the 5′ end and the last three nucleotides at the 3′ end of the HDR repair template polynucleotide. In one aspect, one or more of the components of the system are detectably labeled.

The three nucleotides at the terminal 5′ and 3′ ends of a single stranded HDR template polynucleotide comprises modified nucleotides in each template of the instant disclosure. In some embodiments, the nucleotides at the terminal of the 5′ and 3′ ends are modified nucleotides wherein the template polynucleotide further comprises between the 5′end and the 3′ end of the single stranded HDR template polynucleotide comprises a chemically modified nucleotide at every 4 nucleotides. In some embodiments, the nucleotides at the terminal of the 5′ and 3′ ends are modified nucleotides wherein the template polynucleotide further comprises between the 5′end and the 3′ end of the single stranded HDR template polynucleotide comprises a chemically modified nucleotide at every 5 nucleotides. In some embodiments, the nucleotides at the terminal of the 5′ and 3′ ends are modified nucleotides wherein the template polynucleotide further comprises between the 5′end and the 3′ end of the single stranded HDR template polynucleotide comprises a chemically modified nucleotide at every 6 nucleotides. In some embodiments, the nucleotides at the terminal of the 5′ and 3′ ends are modified nucleotides wherein the template polynucleotide further comprises between the 5′end and the 3′ end of the single stranded HDR template polynucleotide comprises a chemically modified nucleotide at every 7 nucleotides. In some embodiments, the nucleotides at the terminal of the 5′ and 3′ ends are modified nucleotides wherein the template polynucleotide further comprises between the 5′end and the 3′ end of the single stranded HDR template polynucleotide comprises a chemically modified nucleotide at every 8 nucleotides. In some embodiments, the nucleotides at the terminal of the 5′ and 3′ ends are modified nucleotides wherein the template polynucleotide further comprises between the 5′end and the 3′ end of the single stranded HDR template polynucleotide comprises a chemically modified nucleotide at every 9 nucleotides. In some embodiments, the nucleotides at the terminal of the 5′ and 3′ ends are modified nucleotides wherein the template polynucleotide further comprises between the 5′ end and the 3′ end of the single stranded HDR template polynucleotide comprises a chemically modified nucleotide at every 10 nucleotides.

In some embodiments, between about 10% and 25% of the nucleotides between the 5′end and the 3′ end (and optionally including the 5′ and 3′ ends) of the single stranded HDR template polynucleotide comprises a chemically modified nucleotide, wherein the distribution of the modified nucleotides is not fewer than one nucleotide in 10 nucleotides and not more than one nucleotide in 4 nucleotides. In some embodiments, between about 11.1% and 25% of the nucleotides between the 5′end and the 3′ end (and optionally including the 5′ and 3′ ends) of the single stranded HDR template polynucleotide comprises a chemically modified nucleotide, wherein the distribution of the modified nucleotides is not fewer than one nucleotide in 9 nucleotides and not more than one nucleotide in 4 nucleotides. In some embodiments, between about 12.5% and 25% of the nucleotides between the 5′end and the 3′ end (and optionally including the 5′ and 3′ ends) of the single stranded HDR template polynucleotide comprises a chemically modified nucleotide, wherein the distribution of the modified nucleotides is not fewer than one nucleotide in 8 nucleotides and not more than one nucleotide in 4 nucleotides. In some embodiments, between about 14.2% and 25% of the nucleotides between the 5′end and the 3′ end (and optionally including the 5′ and 3′ ends) of the single stranded HDR template polynucleotide comprises a chemically modified nucleotide, wherein the distribution of the modified nucleotides is not fewer than one nucleotide in 7 nucleotides and not more than one nucleotide in 4 nucleotides. In some embodiments, between about 16.6% and 25% of the nucleotides between the 5′ end and the 3′ end (and optionally including the 5′ and 3′ ends) of the single stranded HDR template polynucleotide comprises a chemically modified nucleotide, wherein the distribution of the modified nucleotides is not fewer than one nucleotide in 6 nucleotides and not more than one nucleotide in 4 nucleotides. In some embodiments, between about 20% of the nucleotides between the 5′end and the 3′ end (and optionally including the 5′ and 3′ ends) of the single stranded HDR template polynucleotide comprises a chemically modified nucleotide, wherein the distribution of the modified nucleotides is not fewer than one nucleotide in 8 nucleotides and not more than one nucleotide in 4 nucleotides. In some embodiments, between about 20% and 25% of the nucleotides between the 5′end and the 3′ end (and optionally including the 5′ and 3′ ends) of the single stranded HDR template polynucleotide comprises a chemically modified nucleotide, wherein the distribution of the modified nucleotides is not fewer than one nucleotide in 5 nucleotides and not more than one nucleotide in 4 nucleotides. In some embodiments, between about 10% and 16.6% of the nucleotides between the 5′end and the 3′ end (and optionally including the 5′ and 3′ ends) of the single stranded HDR template polynucleotide comprises a chemically modified nucleotide, wherein the distribution of the modified nucleotides is not fewer than one nucleotide in 10 nucleotides and not more than one nucleotide in 6 nucleotides.

In some embodiments, the single stranded HDR template comprises a transgene. In some embodiments, the integration of the transgene into the target sequence results in inactivation of the target sequence (i.e., the target sequence is disrupted by the inserted transgene, effectively knocking out its expression).

In some embodiments, the HDR repair template polynucleotide is between about 20 and about 10000 bases in length. In some embodiments, the HDR repair template polynucleotide is between about 20 and about 500 bases in length. In some embodiments, the HDR repair template polynucleotide is between about 1000 and about 10000 bases in length. In some embodiments, the HDR repair template polynucleotide is between about 1500 and about 5000 bases in length. In some embodiments, the HDR repair template polynucleotide is between 2000-4000 bases in length. In some embodiments, the HDR repair template polynucleotide is between about 8000 and about 10000 bases in length.

In another aspect, the HDR template is detectably labeled. In some embodiments, the detectable label comprises one or more fluorescent dyes, including but not limited to fluorescein, rhodamine, Alexa Fluors, DyLight fluors, ATTO Dyes, or any analogs or derivatives thereof.

In some embodiments, labels of the present disclosure include but are not limited to fluorescein and chemical derivatives of fluorescein; Eosin; Carboxyfluorescein; Fluorescein isothiocyanate (FITC); Fluorescein amidite (FAM); Erythrosine; Rose Bengal; fluorescein secreted from the bacterium Pseudomonas aeruginosa; Methylene blue; Laser dyes; Rhodamine dyes (e.g., Rhodamine, Rhodamine 6G, Rhodamine B, Rhodamine 123, Auramine O, Sulforhodamine 101, Sulforhodamine B, and Texas Red).

In some embodiments, labels of the present disclosure include but are not limited to ATTO dyes; Acridine dyes (e.g., Acridine orange, Acridine yellow); Alexa Fluor; 7-Amino actinomycin D; 8-Anilinonaphthalene-1-sulfonate; Auramine-rhodamine stain; Benzanthrone; 5,12-Bis(phenylethynyl) naphthacene; 9,10-Bis(phenylethynyl) anthracene; Blacklight paint; Brainbow; Calcein; Carboxyfluorescein; Carboxyfluorescein diacetate succinimidyl ester; Carboxyfluorescein succinimidyl ester; 1-Chloro-9,10-bis(phenylethynyl) anthracene; 2-Chloro-9,10-bis(phenylethynyl) anthracene; 2-Chloro-9,10-diphenylanthracene; Coumarin; Cyanine dyes (e.g., Cyanine such as Cy3 and Cy5, DiOC6, SYBR Green I); DAPI, Dark quencher, DyLight Fluor, Fluo-4, FluoProbes; Fluorone dyes (e.g., Calcein, Carboxyfluorescein, Carboxyfluorescein diacetate succinimidyl ester, Carboxyfluorescein succinimidyl ester, Eosin, Eosin B, Eosin Y, Erythrosine, Fluorescein, Fluorescein isothiocyanate, Fluorescein amidite, Indian yellow, Merbromin); Fluoro-Jade stain; Fura-2; Fura-2-acetoxymethyl ester; Green fluorescent protein, Hoechst stain, Indian yellow, Indo-1, Lucifer yellow, Luciferin, Merocyanine, Optical brightener, Oxazin dyes (e.g., Cresyl violet, Nile blue, Nile red); Perylene; Phenanthridine dyes (Ethidium bromide and Propidium iodide); Phloxine, Phycobilin, Phycoerythrin, Phycoerythrobilin, Pyranine, Rhodamine, Rhodamine 123, Rhodamine 6G, RiboGreen, RoGFP, Rubrene, SYBR Green I, (E)-Stilbene, (Z)-Stilbene, Sulforhodamine 101, Sulforhodamine B, Synapto-pHluorin, Tetraphenyl butadiene, Tetrasodium tris(bathophenanthroline disulfonate) ruthenium (II), Texas Red, TSQ, Umbelliferone, or Yellow fluorescent protein.

In some embodiments, labels of the present invention disclosure but are not limited to Alexa Fluor family of fluorescent dyes (Molecular Probes, Oregon). Alexa Fluor dyes are widely used as cell and tissue labels in fluorescence microscopy and cell biology. The excitation and emission spectra of the Alexa Fluor series cover the visible spectrum and extend into the infrared. The individual members of the family are numbered according roughly to their excitation maxima (in nm). Certain Alexa Fluor dyes are synthesized through sulfonation of coumarin, rhodamine, xanthene (such as fluorescein), and cyanine dyes. In some embodiments, sulfonation makes Alexa Fluor dyes negatively charged and hydrophilic. In some embodiments, Alexa Fluor dyes are more stable, brighter, and less pH-sensitive than common dyes (e.g. fluorescein, rhodamine) of comparable excitation and emission, and to some extent the newer cyanine series. Exemplary Alexa Fluor dyes include but are not limited to Alexa-350, Alexa-405, Alexa-430, Alexa-488, Alexa-500, Alexa-514, Alexa-532, Alexa-546, Alexa-555, Alexa-568, Alexa-594, Alexa-610, Alexa-633, Alexa-647, Alexa-660, Alexa-680, Alexa-700, or Alexa-750.

In some embodiments, labels of the present disclosure comprise one or more of the DyLight Fluor family of fluorescent dyes (Dyomics and Thermo Fisher Scientific). Exemplary DyLight Fluor family dyes include but are not limited to DyLight-350, DyLight-405, DyLight-488, DyLight-549, DyLight-594, DyLight-633, DyLight-649, DyLight-680, DyLight-750, or DyLight-800.

In some embodiments, a detectable label is or comprises a nanomaterial. In some embodiments, a detectable label is or compresses a nanoparticle. In some embodiments, a detectable label is or comprises a quantum dot. In some embodiments, a detectable label is a quantum dot. In some embodiments, a detectable moiety comprises a quantum dot. In some embodiments, a detectable label is or comprises a gold nanoparticle. In some embodiments, a detectable label is a gold nanoparticle. In some embodiments, a detectable label comprises a gold nanoparticle.

In some embodiments, the single stranded HDR template comprises a template to correct a single point mutation in the target sequence.

In some embodiments, the chemically-modified nucleotide comprises a modification has been shown to make ssDNA resistant to nuclease activity. In some embodiments, the nucleotide modification comprises a phosphorothioate (PS) modification, a 2′ o-methyl modification, methoxypropyl phosphonate (MOP), a constrained ethyl (cEt) modification, a locked nucleic acid (LNA) modification, a 2′ fluoro modification, a 1-Boranotriphosphate modification, methoxypropyl phosphonate (MOP), phosphorodiamidate morpholino oligomer (PMO), or a combination thereof. In some embodiments, the nucleotide modification comprises one or more of the modifications shown in FIGS. 4A-4B.

In some embodiments, the single stranded HDR template is a single stranded DNA. In some embodiments, the single stranded HDR template is a single stranded RNA/DNA hybrid polynucleotide.

In some embodiments, the gene-editing system comprises a CRISPR system. In some embodiments, the CRISPR system comprises a CRISPR enzyme. In some embodiments, the CRISPR system comprises a polynucleotide encoding a CRISPR enzyme. In some embodiments, the CRISPR system further comprises a guide RNA (gRNA) sequence that hybridizes to a target sequence, a tracr mate sequence, a tracr sequence. In some embodiments, the CRISPR enzyme is a type II CRISPR-Cas enzyme. In some embodiments, the type II CRISPR-Cas enzyme is a Cas9 enzyme. In some embodiments, the Cas9 enzyme is a Streptococcus pyogenes Cas9 (SpCas9) enzyme or a Staphylococcus aureus Cas9 (SaCas9) enzyme. In a further aspect, one or more components of the CRISPR system are detectably labeled.

In some embodiments, the CRISPR enzyme is a high-fidelity enzyme. In some embodiments, the high-fidelity enzyme is a Cas9 enzyme having a mutation at a position corresponding to R691 of SEQ ID NO: 1. In a specific embodiment, the Cas9 comprises a R691A mutation.

In some embodiments, the gene-editing system comprises a nickase. In some embodiments, the CRISPR enzyme is a Cas9 enzyme having one or more mutations at a position corresponding to D10A, E762A, H840A, N854A, N863A or D986A of SEQ ID NO: 1. In some embodiments, the Cas9 mutation is selected from D10A, E762A, H840A, N854A, N863A or D986A. In some embodiments, the one or more mutations is in a RuvC1 domain of the Cas9 (corresponding to amino acids 1 to 60 of SEQ ID NO: 1). In some embodiments, the CRISPR enzyme comprises one or more mutations corresponding to one or more mutations selected from D10A, E762A, H840A, N854A, N863A or D986A (numbering according to SEQ ID NO: 1), or wherein the one or more mutations is in a RuvC1 domain of the CRISPR enzyme (corresponding to the amino acids 1 to 60 of SEQ ID NO: 1).

In some embodiments, the gene-editing system is a CRISPR system comprising, or consisting essentially of, or consisting of a CRISPR enzyme or a polynucleotide encoding a CRISPR enzyme, a guide RNA (gRNA) sequence that hybridizes to a target sequence, a tracr mate sequence, a tracr sequence, and a second guide RNA sequence that hybridizes to a second target sequence, wherein the guide RNA sequence directs cleavage of one strand of the DNA duplex near the target sequence and the second guide RNA sequence directs cleavage of other strand near the second target sequence inducing a double strand break.

In some embodiments, the CRISPR enzyme comprises at least one nuclear localization sequence. In some embodiments, the CRISPR enzyme comprises two nuclear localization sequences. In some embodiments, the NLS comprises a sequence selected from SEQ ID NOs: 2-17.

In some embodiments, the gene-editing system comprises a TALEN system comprising, or consisting essentially of, or consisting of, from the N-terminus to the C-terminus, a first spacer sequence, a TALE DNA binding domain, a second spacer sequence, and a FokI nuclease catalytic domain fused to the C-terminus, wherein the TALE DNA binding domain comprises TAL repeats, wherein the TALE DNA binding domain is specific for binding to target sequence.

In some embodiments, the gene-editing system comprises a ZFN system comprising, or consisting essentially of, or consisting of at least one zinc finger nuclease, the zinc finger nuclease comprising a zinc finger protein comprising 4, 5 or 6 fingers designated F1 to F4, F1 to F5 or F1 to F6, each finger comprising a recognition helix region that recognizes a target sequence.

In some embodiments, the gene-editing system is formulated for delivery in a liposome, e.g., a lipid nanoparticle.

### Compositions

Also provided herein are one or more of the non-naturally occurring or engineered composition as disclosed herein or a cell comprising, or consisting essentially of, or consisting of the non-naturally occurring or engineered composition as disclosed herein, and a carrier, such as a pharmaceutically acceptable carrier. As disclosed herein, the non-naturally occurring or engineered system comprises components of a CRISPR-Cas system, a TALEN system and a Zinc Finger Nuclease (ZFN) system. Examples of carriers such as pharmaceutically acceptable carriers are provided herein, and non-limiting examples of such are described herein and include for example, a nanoparticles, an exosome, or a microvesicle.

In a further aspect, provided herein is a composition comprising, or consisting essentially of, or consisting of a plurality of the non-naturally occurring or engineered composition as disclosed herein or a cell comprising the non-naturally occurring or engineered composition as disclosed herein, wherein any or both of the composition is the same or different from each other. An aspect of the disclosure is directed to a non-naturally occurring or engineered composition comprising, or consisting essentially of, or consisting of: (i) a polynucleotide sequence encoding a CRISPR enzyme, (ii) a CRISPR-Cas system chimeric RNA (chiRNA) polynucleotide sequence comprising: (a) a guide sequence capable of hybridizing to a target sequence, (b) a tracr mate sequence, and (c) a tracr sequence, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence; and (ii) a single stranded homology directed repair (HDR) template polynucleotide that comprises a chemically modified nucleotide every 4, 5, 6, 7 or 8 nucleotides along at least 95% (i.e., at least 95%, 96%, 97%, 98%, 99% or more) of the length of the HDR template polynucleotide.

An aspect of the disclosure is directed to a non-naturally occurring or engineered composition comprising, or consisting essentially of, or consisting of: (i) a polynucleotide sequence encoding a CRISPR enzyme, (ii) a CRISPR-Cas system chimeric RNA (chiRNA) polynucleotide sequence comprising: (a) a guide sequence capable of hybridizing to a target sequence, (b) a tracr mate sequence, and (c) a tracr sequence, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence; and (ii) a single stranded homology directed repair (HDR) template polynucleotide that comprises a chemically modified nucleotide every 4, 5, 6, 7, 8, 9 or 10 nucleotides along at least 95% (i.e., at least 95%, 96%, 97%, 98%, 99% or more) of the length of the HDR template polynucleotide.

In some embodiments, the composition further comprises a carrier, such as a pharmaceutically acceptable carrier, such as for example, a liposome (e.g., a lipid nanoparticle), a nanoparticle, an exosome, and/or a microvesicle. In some embodiments, the at least 95% (at least 95%, 96%, 97%, 98%, 99% or more) includes the three modified nucleotides at the 5′ and 3′ ends of the HDR template. In a further aspect, one or more components of the CRISPR system in the composition are detectably labeled.

In some embodiments, any or all of the polynucleotide sequence encoding the CRISPR enzyme, the guide sequence, the tracr mate sequence or the tracr sequence, is/are DNA.

In some embodiments, the any one or all of the polynucleotide sequence encoding the CRISPR enzyme, the guide sequence, the tracr mate sequence or the tracr sequence further comprise a vector and at least one regulatory element operationally linked to the polynucleotide sequence.

In some embodiments, any or all of the polynucleotide sequence encoding the CRISPR enzyme, the guide sequence, the tracr mate sequence or the tracr sequence, is/are RNA.

In some embodiments, the single stranded HDR template nucleic acid is a single stranded DNA. In some embodiments, the single stranded HDR template nucleic acid is a single stranded RNA.

In some embodiments, the CRISPR enzyme is a type II CRISPR-Cas enzyme, optionally wherein the CRISPR enzyme is a Cas9 enzyme. In some embodiments, the CRISPR enzyme is an SpCas9 enzyme or an SaCas9 enzyme.

In some embodiments, the CRISPR enzyme is a high-fidelity enzyme. In some embodiments, the high-fidelity enzyme is a Cas9 enzyme having a mutation at a position corresponding to R691 of SEQ ID NO: 1. In a specific embodiment, the Cas9 comprises a R691A mutation.

In some embodiments, the CRISPR enzyme is a nickase.

In some embodiments, the CRISPR enzyme is a Cas9 enzyme having one or more mutations at a position corresponding to D10A, E762A, H840A, N854A, N863A or D986A of SEQ ID NO: 1. In some embodiments, the Cas9 mutation is selected from D10A, E762A, H840A, N854A, N863A or D986A. In some embodiments, the one or more mutations is in a RuvC1 domain of the Cas9 (corresponding to amino acids 1 to 60 of SEQ ID NO: 1). In some embodiments, the CRISPR enzyme comprises one or more mutations corresponding to one or more mutations selected from D10A, E762A, H840A, N854A, N863A or D986A (numbering according to SEQ ID NO: 1), or wherein the one or more mutations is in a RuvC1 domain of the CRISPR enzyme (corresponding to the amino acids 1 to 60 of SEQ ID NO: 1).

In some embodiments, the composition further comprises: (iv) a second CRISPR-Cas system chimeric RNA (chiRNA) polynucleotide sequence comprising, or consisting essentially of, or consisting of: (a) a second guide sequence capable of hybridizing to a second target sequence, (b) a second tracr mate sequence, and (c) a second tracr sequence, wherein (a), (b) and (c) are arranged in a 5′ to 3′ orientation, and wherein when transcribed, the second tracr mate sequence hybridizes to the second tracr sequence and the second guide sequence directs sequence-specific binding of a second CRISPR complex to the second target sequence.

In some embodiments, the CRISPR enzyme comprises at least one nuclear localization sequence, optionally wherein the CRISPR enzyme comprises two nuclear localization sequences.

In some embodiments, the composition further comprises a TREX1 inhibitor.

In some embodiments, the composition of the present disclosure is formulated for delivery in a liposome such as a lipid nanoparticle.

### Methods

Another aspect of the disclosure is directed to a method for modifying an organism or a non-human organism comprising, or consisting essentially of, or consisting of delivering the gene-editing system of the instant disclosure to the organism or the non-human organism. The gene-editing system can be delivered in a composition or in a host cell. Modes of delivery or administration are known in the art and include locally or systemically, e.g., intravenously. When delivered via a host cell, the cell can be a mammalian or human cell. In a yet further aspect, the cell is a mammalian or human stem cell. In a yet further aspect, the mammalian or human cell is autologous or allogenic to an organism that is the intended recipient of the edited cell. The methods are useful for therapy or for assaying new therapies or combination therapies for the treatments of animals and human patients. One of skill in the art will know when the therapy is successful by noting a reduction or alleviation of symptoms of the underlying disease being treated.

Another aspect of the disclosure is directed to a method for modifying an organism or a non-human organism comprising delivering the composition of the instant disclosure comprising a gene-editing system to the organism or the non-human organism. Modes of delivery or administration are known in the art and include locally or systemically, e.g., intravenously. When delivered via a host cell, the cell can be a mammalian or human cell. In a yet further aspect, the cell is a mammalian or human stem cell. In a yet further aspect, the mammalian or human cell is autologous or allogenic to an organism that is the intended recipient of the edited cell. The methods are useful for therapy or for assaying new therapies or combination therapies for the treatments of animals and human patients. One of skill in the art will know when the therapy is successful by noting a reduction or alleviation of symptoms of the underlying disease being treated.

Another aspect of the disclosure is directed to a method for modifying the genome of a cell comprising delivering the gene-editing system or the composition of the instant disclosure.

In some embodiments, the disclosure is directed to a method for modifying the CTFR gene in a subject in need thereof. In some embodiments, the subject is suffering from cystic fibrosis.

In some embodiments, the CTFR gene has a G551D mutation.

On some embodiments, the CTFR gene has a F508del mutation. In some embodiments, the HDR template nucleic acid comprises a sequence as shown in SEQ ID NO: 38 or 40. In some embodiments, the HDR template nucleic acid comprises a sequence as shown in SEQ ID NO: 38, wherein three nucleotides at both ends are chemically modified and that every 4^{th} internal nucleotide between the ends is also chemically modified. In some embodiments, the HDR template nucleic acid comprises a sequence as shown in SEQ ID NO: 38, wherein three nucleotides at both ends are chemically modified and that every 5^{th} internal nucleotide between the ends is also chemically modified. In some embodiments, the HDR template nucleic acid comprises a sequence as shown in SEQ ID NO: 38, wherein three nucleotides at both ends are chemically modified and that every 5^{th} internal nucleotide between the ends is also chemically modified. In some embodiments, the HDR template nucleic acid comprises a sequence as shown in SEQ ID NO: 38, wherein three nucleotides at both ends are chemically modified and that every 6^{th} internal nucleotide between the ends is also chemically modified. In some embodiments, the HDR template nucleic acid comprises a sequence as shown in SEQ ID NO: 38, wherein three nucleotides at both ends are chemically modified and that every 7^{th} internal nucleotide between the ends is also chemically modified. In some embodiments, the HDR template nucleic acid comprises a sequence as shown in SEQ ID NO: 38, wherein three nucleotides at both ends are chemically modified and that every 8^{th} internal nucleotide between the ends is also chemically modified. In some embodiments, the HDR template nucleic acid comprises a sequence as shown in SEQ ID NO: 38, wherein three nucleotides at both ends are chemically modified and that every 9^{th} internal nucleotide between the ends is also chemically modified. In some embodiments, the HDR template nucleic acid comprises a sequence as shown in SEQ ID NO: 38, wherein three nucleotides at both ends are chemically modified and that every 10^{th} internal nucleotide between the ends is also chemically modified.

In some embodiments, a chemically modified nucleotide in SEQ ID NO: 38 comprises a modification has been shown to make ssDNA resistant to nuclease activity. In some embodiments, a chemically modified nucleotide in SEQ ID NO: 38 comprises a phosphorothioate (PS) modification, a 2′-o-methyl modification, methoxypropyl phosphonate (MOP), a constrained ethyl (cEt) modification, a locked nucleic acid (LNA) modification, a 2′ fluoro modification, a 1-Boranotriphosphate modification, methoxypropyl phosphonate (MOP) modification, phosphorodiamidate morpholino oligomer (PMO) modification, or a combination thereof. In some embodiments, a chemically modified nucleotide in SEQ ID NO: 38 comprises a phosphorothioate (PS) modification.

In some embodiments, the method further comprises administering to the organism or the cell with an effective amount of a TREX1 inhibitor.

In some embodiments, the method further comprises administering to the organism or the cell with an effective amount of a DNA-PKcs inhibitor. In some embodiments, the DNA-PKcs inhibitor is selected from AZD7648, M3814 (peposertib), M9831 (VX-984) or BAY-8400.

In some embodiments, the gene-editing system or the composition is delivered in vitro or in vivo. In some embodiments, the cell is a prokaryotic cell or a eukaryotic cell. In a further aspect, the cell is a mammalian or human cell. In a yet further aspect, the cell is a mammalian or human stem cell. In a yet further aspect, the mammalian or human cell is autologous or allogenic to an organism that is the intended recipient of the edited cell.

In some embodiments, the gene-editing system or the composition is delivered via nanoparticles (e.g., lipid nanoparticles), exosomes, microvesicles, electroporation or a gene-gun.

Another aspect of the disclosure is directed to a method for ex vivo cell therapy comprising administering a subject a cell prepared by one of the methods of the instant disclosure. The cell can be a mammalian or human cell. In a yet further aspect, the cell is a mammalian or human stem cell. In a yet further aspect, the mammalian or human cell is autologous or allogenic to an organism that is the intended recipient of the edited cell. The methods are useful for therapy or for assaying new therapies or combination therapies for the treatments of animals and human patients. One of skill in the art will know when the therapy is successful by noting a reduction or alleviation of symptoms of the underlying disease being treated.

In some embodiments, a cell used in ex vivo cell therapy is autologous to a subject being treated, i.e., the cell is derived from the subject, genetically modified using the gene-editing systems or compositions disclosed herein and administered back to the subject. In some embodiments, a cell used in ex vivo cell therapy is heterologous to a subject being treated, i.e., the cell is derived from a different donor, genetically modified using the gene-editing systems or compositions disclosed herein and administered to the subject.

In some embodiments, the cell is a mammalian cell. In some embodiments, the cell is a human cell. In some embodiments, the cell is a non-human cell.

In some embodiments, the cell is an airway cell. In a further aspect, the cell is a stem cell. In a further aspect, the cell is an airway stem cell. In a separate aspect, the cell is a human umbilical vein endothelial cell (“HUVEC”). In a separate aspect, the cell is a Hematopoietic Stem and Progenitor cell (“HSPC”).

In some embodiments, the composition of the instant disclosure comprises a non-naturally occurring or engineered composition comprising: (i) a polynucleotide sequence encoding a CRISPR enzyme, (ii) a CRISPR-Cas system chimeric RNA (chiRNA) polynucleotide sequence comprising: (a) a guide sequence capable of hybridizing to a target sequence, (b) a tracr mate sequence, and (c) a tracr sequence, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence; and (ii) a single stranded homology directed repair (HDR) template polynucleotide that comprises a chemically modified nucleotide every 4, 5, 6, 7 or 8 nucleotides along at least 95% (at least 95%, at least 96%, at least 97%, at least 98%, at least 99% or more) of the length of the HDR template polynucleotide. In some embodiments, the at least 95% includes the three modified nucleotides at the 5′ and 3′ ends of the HDR template.

In some embodiments, the composition of the instant disclosure comprises a non-naturally occurring or engineered composition comprising: (i) a polynucleotide sequence encoding a CRISPR enzyme, (ii) a CRISPR-Cas system chimeric RNA (chiRNA) polynucleotide sequence comprising: (a) a guide sequence capable of hybridizing to a target sequence, (b) a tracr mate sequence, and (c) a tracr sequence, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence; and (ii) a single stranded homology directed repair (HDR) template polynucleotide that comprises a chemically modified nucleotide every 4, 5, 6, 7, 8, 9 or 10 nucleotides along at least 95% (at least 95%, at least 96%, at least 97%, at least 98%, at least 99% or more) of the length of the HDR template polynucleotide. In some embodiments, the at least 95% includes the three modified nucleotides at the 5′ and 3′ ends of the HDR template.

In some embodiments, the composition of the instant disclosure comprises a non-naturally occurring or engineered composition comprising: (i) a polynucleotide sequence encoding a CRISPR enzyme, (ii) a CRISPR-Cas system chimeric RNA (chiRNA) polynucleotide sequence comprising: (a) a guide sequence capable of hybridizing to a target sequence, (b) a tracr mate sequence, and (c) a tracr sequence, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence; and (ii) a single stranded homology directed repair (HDR) template polynucleotide that comprises a chemically modified nucleotide every 6 nucleotides along the entire length of the HDR template polynucleotide, and optionally wherein the three nucleotides at the 5′ and 3′ ends of the HDR template are also modified.

In some embodiments, the composition of the instant disclosure comprises a non-naturally occurring or engineered composition comprising: (i) a polynucleotide sequence encoding a CRISPR enzyme, (ii) a CRISPR-Cas system chimeric RNA (chiRNA) polynucleotide sequence comprising: (a) a guide sequence capable of hybridizing to a target sequence, (b) a tracr mate sequence, and (c) a tracr sequence, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence; and (ii) a single stranded homology directed repair (HDR) template polynucleotide that comprises a chemically modified nucleotide every 7 nucleotides along the entire length of the HDR template polynucleotide, and optionally wherein the three nucleotides at the 5′ and 3′ ends of the HDR template are also modified.

In some embodiments, the composition of the instant disclosure comprises a non-naturally occurring or engineered composition comprising: (i) a polynucleotide sequence encoding a CRISPR enzyme, (ii) a CRISPR-Cas system chimeric RNA (chiRNA) polynucleotide sequence comprising: (a) a guide sequence capable of hybridizing to a target sequence, (b) a tracr mate sequence, and (c) a tracr sequence, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence; and (ii) a single stranded homology directed repair (HDR) template polynucleotide that comprises a chemically modified nucleotide every 8 nucleotides along the entire length of the HDR template polynucleotide, and optionally wherein the three nucleotides at the 5′ and 3′ ends of the HDR template are also modified.

In some embodiments, the composition of the instant disclosure comprises a non-naturally occurring or engineered composition comprising: (i) a polynucleotide sequence encoding a CRISPR enzyme, (ii) a CRISPR-Cas system chimeric RNA (chiRNA) polynucleotide sequence comprising: (a) a guide sequence capable of hybridizing to a target sequence, (b) a tracr mate sequence, and (c) a tracr sequence, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence; and (ii) a single stranded homology directed repair (HDR) template polynucleotide that comprises a chemically modified nucleotide every 9 nucleotides along the entire length of the HDR template polynucleotide, and optionally wherein the three nucleotides at the 5′ and 3′ ends of the HDR template are also modified.

In some embodiments, the composition of the instant disclosure comprises a non-naturally occurring or engineered composition comprising: (i) a polynucleotide sequence encoding a CRISPR enzyme, (ii) a CRISPR-Cas system chimeric RNA (chiRNA) polynucleotide sequence comprising: (a) a guide sequence capable of hybridizing to a target sequence, (b) a tracr mate sequence, and (c) a tracr sequence, wherein (a), (b) and (c) are arranged in a 5′ to 3′ orientation, wherein when transcribed, the tracr mate sequence hybridizes to the tracr sequence and the guide sequence directs sequence-specific binding of a CRISPR complex to the target sequence; and (ii) a single stranded homology directed repair (HDR) template polynucleotide that comprises a chemically modified nucleotide every 10 nucleotides along the entire length of the HDR template polynucleotide, and optionally wherein the three nucleotides at the 5′ and 3′ ends of the HDR template are also modified.

Another aspect of the disclosure is directed to a method comprising delivering the gene-editing system or the composition of the instant disclosure to a cell, producing clones of the cell, and screening for the clones with altered biological activity (e.g., identifying mutations that may be oncogenic) or assessing the pathogenicity of mutations suspected to be involved in a disease process (e.g., assessing whether a particular mutation is capable of causing cystic fibrosis). The following examples are provided to illustrate but not limit the scope of this disclosure.

### EXAMPLES

### Example 1: Chemically Modified ssDNA Improves Gene Editing in Cells

Methods. 4 nmol single stranded DNA (ssDNA) ultramers were ordered from Integrated DNA Technologies “IDT” (idtdna.com/pages/products/custom-dna-rna/dna-oligos/ultramer-dna-oligos, last accessed Apr. 7, 2024). In the assays, following ssDNA combinations were tested (i) 40 bp homology arms (HA) (“short HA”), and (ii) 80-100 bp HA (“long HA”). The short and long HA ssDNAs comprised central termination sequences (CTS sequences). In addition, the ssDNAs were produced ends modified with phosphorothioate (PS), every sixth base modified with PS, or every third base modified with PS.

The following sequences are based on Vaidyanathan, Sriram, et al. (Cell Stem Cell 26.2 (2020): 161-171) for correcting the AF508 mutation of CFTR (cystic fibrosis transmembrane conductance regulator), which is a mutation commonly found in cystic fibrosis patients.

SV150-ssDNA-short HA (SVU1):
(SEQ ID NO: 34)
5′CCTGGATTATGCCTGGCACCATTAAAGAAAATATCATCTTcGGcGTgT
CtTAcGAcGAgTAcAGATACAGAAGCGTCATCAAAGCATGCCAACTAGAA
GAGG-3′
End mismatch(SVU2):
(SEQ ID NO: 35)
5′ATGCTATCTATATTCATCATAGGCCTGGATTATGCCTGGCACCATTAA
AGAAAATATCATCTTcGGcGTgTCtTAcGAcGAgTAcAGATACAGAAGCG
TCATCAAAGCATGCCAACTAGAAGAGGCCTATGATGAATATAGATAGC
AT-3′
SVO1
(SEQ ID NO: 36)
5′-ATGCTATCTATATTCATCATAGGCCTCTTCTAG-3′
SVO2
(SEQ ID NO: 37)
5′-ATAATCCAGGCCTATGATGAATATAGATAGCAT-3′
SV150-ssDNA-Unmodified Long HA (SVU3)
(SEQ ID NO: 38)
ATGGGAGAACTGGAGCCTTCAGAGGGTAAAATTAAGCACAGTGGAAGAAT
TTCATTCTGTTCTCAGTTTTCCTGGATTATGCCTGGCACCATTAAAGAAA
ATATCATCTTcGGcGTgTCtTAcGAcGAgTAcAGATACAGAAGCGTCATC
AAAGCATGCCAACTAGAAGAGGTAAGAAACTATGTGAAAACTTTTTGATT
SV150-ssDNA-End modified (SVU4)
(SEQ ID NO: 39)
A*T*G*GGAGAACTGGAGCCTTCAGAGGGTAAAATTAAGCACAGTGGAAG
AATTTCATTCTGTTCTCAGTTTTCCTGGATTATGCCTGGCACCATTAAAG
AAAATATCATCTTcGGcGTgTCtTAcGAcGAgTAcAGATACAGAAGCGTC
ATCAAAGCATGCCAACTAGAAGAGGTAAGAAACTATGTGAAAACTTTTT
G*A*T*T (*indicates that the preceding nucleotide
is PS modified)
SV150-Internal mods - Every sixth (SVU5)
(SEQ ID NO: 40)
A*T*G*GGAGAA*CTGGAG*CCTTCA*GAGGGT*AAAATT*AAGCAC*AG
TGGA*AGAATT*TCATTC*TGTTCT*CAGTTT*TCCTGG*ATTATG*CCT
GGC*ACCATT*AAAGAA*AATATC*ATCTTc*GGcGTg*TCtTAc*GAcG
Ag*TAcAGA*TACAGA*AGCGTC*ATCAAA*GCATGC*CAACTA*GAAGA
G*GTAAGA*AACTAT*GTGAAA*ACTTTT*TG*A*T*T
(* indicates that the preceding nucleotide is PS
modified)
SV150-Internal mods - Every third (SVU6)
(SEQ ID NO: 41)
A*T*G*GGA*GAA*CTG*GAG*CCT*TCA*GAG*GGT*AAA*ATT*AAG*
CAC*AGT*GGA*AGA*ATT*TCA*TTC*TGT*TCT*CAG*TTT*TCC*
TGG*ATT*ATG*CCT*GGC*ACC*ATT*AAA*GAA*AAT*ATC*ATC*
TTc*GGc*GTg*TCt*TAc*GAc*GAg*TAc*AGA*TAC*AGA*AGC*
GTC*ATC*AAA*GCA*TGC*CAA*CTA*GAA*GAG*GTA*AGA*AAC*
TAT*GTG*AAA*ACT*TTT*TG*A*T*T
(* indicates that the preceding nucleotide is
PS modified)

Generation of ssDNA templated with CTS. Similar to other ssDNA templates, the longer ssDNA template was ordered as Ultramers from Integrated DNA Technologies. The template and oligos were resuspended in IDT buffer. The short oligos were mixed with long ssDNA at 4:1 molar ratio and then heated to 95 C and cooled by 5 C per 5 mins (1 C per min) in a thermocycler. The annealed product was at a concentration of 8 μM and was used in experiments at a concentration of 800 nM.

Cell Culture of ABCs. ABCs are airway epithelial cells derived either from a CF patient at the time of lung transplant or from cadaveric wild-type (wt) tissues. These cells are usually expanded in tissue culture before plating them on permeable supports or growing them at an air-liquid interface to mimic the native airway environment. Human ABCs were cultured in PneumaCult Ex Plus medium (StemCell Technologies #05040, stemcell.com/products/pneumacult-ex-plus-medium.html) with addition of ROCK inhibitor 10 μM (Y-27632, MedChemExpress #HY-10583, medchemexpress.com/search.html?q=HY-10583&ft=&fa=&fp=&type-inhibitors-and-agonists, last accessed Apr. 5, 2024) and iMatrix-511 (Nacalai USA #892021, nacalaiusa.com/products/view/354/imatrix-511, last accessed Apr. 5, 2024) at 5000-10,000 cells/cm² in tissue culture flasks.

Cell Culture of HSPCs. G-CSF Mobilized Human Peripheral Blood CD34+ Cells were purchased from StemCell Technologies (Catalog: 70060.1, stemcell.com/mobilized-human-peripheral-blood-cd34-cells-frozen.html, last accessed Apr. 4, 2024). Cells were thawed by resuspending in 10 mL of RPMI without FBS. The tube was centrifuged at 300 g for 10 minutes and the supernatant removed. Cells were resuspended in StemSpan™ SFEM II (which comprises Iscove's Modified Dulbecco's Medium (MDM) with 2% Fetal Bovine Serum (FBS), Bovine serum albumin, Recombinant human insulin, Human transferrin (iron-saturated), 2-Mercaptoethanol) supplemented with StemSpan™ CD34+ Expansion Supplement (10×) (which comprises Recombinant human fms-like tyrosine kinase 3 ligand (Flt3L), Recombinant human stem cell factor (SCF), Recombinant human interleukin 3 (IL-3), Recombinant human interleukin 6 (IL-6), Recombinant human thrombopoietin (TPO)) (StemCell Technologies, Catalog #09720, stemcell.com/products/stemspan-cd34-expansion-supplement-10x.html, last accessed Apr. 5, 2024). Cells were plated on 24-well non-treated plates at a density of 100,000 cells/mL and incubated at 37° C. for 2-3 days.

Cell Culture of iPSCs. Normal human induced pluripotent stem cells (Gibco, A18945) were cultured in StemFlex™ media (ThermoFisher Scientific, A3349401) at a density of 6.0×104 cells/cm² on plates coated with vitronectin (ThermoFisher Scientific, A31804) and incubated at 37° C. for 2 days. The ROCK inhibitor, Y-27632, was added to the media 1 h before the experiment at a concentration of 10 μM. Cells were electroporated 2-3 days after plating. After electroporation, cells were plated and cultured for 3-4 days after which genomic DNA was extracted.

Cell Culture of HUVECs. Primary Umbilical Vein Endothelial Cells were purchased from American Type Culture Collection (ATCC, PCS100010, atcc.org/products/pcs-100-010, last accessed Apr. 5, 2024). Cells were cultured in EGM™-2 Endothelial Cell Growth Medium-2 BulletKit™ (Lonza, CC-3162, lonza.com/lonza_bs/US/en/Primary-and-Stem-Cells/p/000000000000185303/EGM™-2-Endothelial-Cell-Growth-Medium-2-BulletKit™, last accessed Apr. 5, 2024) and plated in T75 culture flask at 5000 cells/cm² with iMatrix-511 (Nacalai USA #892021, nacalaiusa.com/products/view/354/imatrix-511, last accessed Apr. 5, 2024).

Cell Culture of T-cells. Primary T-cells were purchased from StemCell Technologies (#70024, stemcell.com/human-peripheral-blood-pan-t-cells-frozen.html, last accessed Apr. 5, 2024). Cells were cultured in X-Vivo 15 media supplemented with 5% human serum and 10 ng/ml of IL-2. T-cells were activated using Dynabeads™ T-cell activator with CD3/CD28 (Thermofisher Inc., #11161D, thermofisher.com/order/catalog/product/11161D?SID=srch-hj-11161D, last accessed Apr. 5, 2024) for 1-2 days using directions provided by the manufacturer (1:1 bead to cell ratio). Failure to remove beads resulted in reduced cell proliferation. Cells were edited after 4-6 days in culture.

Gene Editing of ABCs, iPSCs, and HUVECs. Electroporation was performed 4-5 days after plating. Cells were suspended by treatment with TrypLE™ Express Enzyme (1×), phenol red (ThermoFisher Scientific, 12605036). Cells were suspended in Opti-MEM™ I Reduced Serum Medium (ThermoFisher Scientific, 31985088) at 2.5-10 million cells/mL. Cells were electroporated with Lonza 4D-Nucleofector™ in 16-well Nucleocuvette Strips (Lonza, V4XP-3032). Cas9 at 6 μg (Integrated DNA Technologies) and sgRNA at 3.2 μg (Synthego) were complexed at room temperature for 10 minutes before adding 100,000 cells (20 μL/well). Cell mixture was transferred to Lonza strip at 20 μL per well and electroporated using program CA-137 with solution setting P3. Afterward, 80 μL of OptiMEM was added to each well. When editing using adenoassociated viral vectors (AAVs), AAVs were added after electroporation. ABCs and HUVECs were treated with 105 vg/cell. iPSCs were treated with 104 vg/cell. Cells were then transferred to a 12 or 24-well tissue culture plate. Pneumacult Ex Plus (StemCell Technologies #05040), ROCK inhibitor 10 μM (Y-27632, MedChem Express HY-10583), and iMatrix-511 (Nacalai USA #892021) were added to ABCs and EGM™-2 Endothelial Cell Growth Medium-2 BulletKit™ (Lonza, CC-3162) with iMatrix was added to HUVECs and then incubated in 37° C. at 5% 02. iPSCs were plated with media described above and incubated at 37° C. AZD-7648 (MedChem Express, HY-111783) was added for DNA-PKcs inhibition at a concentration of 0.5 μM. ART558 (MedChem Express, HY-141520) was added for PolT inhibition at a concentration of 5 M.

Gene Editing of HSPCs and T-cells. Hematopoietic stem and progenitor cells were counted after two days and centrifuged at 300 g for 10 minutes. T-cells were counted after 4-6 days in culture. Supernatant was removed and cells were resuspended in P3 Primary Cell Nucleofector® Solution with Supplement 1 (Lonza, V4XP-3032) at 5-15 million cell/mL. Cell mixture was transferred to Lonza strip at 20 μL per well and electroporated using program DZ100 with solution setting P3. 80 μL of StemSpan™ SFEM II supplemented with StemSpan™ CD34+ Expansion Supplement (10×) (StemCell Technologies, Catalog #09720) was added to each well. AAV was added after electroporation, if necessary, at 104 vg/cell. HSPCs were transferred to 24-well non-treated plates at a density of 100,000 cells/mL and incubated at 37° C. for 2-3 days. T-cells were cultured at densities of 500,000-1,000,000 cells/mL.

Gene Correction Measurement. Approximately 3-4 days after electroporation, genomic DNA was extracted using QuickExtract (QE) DNA Extraction Solution (Biosearch Technologies, QE09050). TrypLE Express Enzyme was added to cells, then after 20 minutes they were centrifuged at 300 g for 10 minutes. Supernatant was removed, cells were suspended in OptiMEM and counted. 50-100 thousand cells were taken, re-centrifuged and the pellet was suspended in 50 μL of QE. The QE suspension was heated at 65° C. for 6 minutes then heated at 98° C. for 10 minutes. Amplification was done with the following primers:

| Gene Loci | Forward Primer | Reverse Primer | Sequencing Primer |
|---|---|---|---|
| F508del | CCTTCTACTCAGTTTT | TGGGTAGTGTGAAGG | AGGCAAGTGAATCCT |
| | AGTC (SEQ ID NO: 42) | GTTCAT (SEQ ID NO: 43) | GAGCG (SEQ ID NO: 44) |
| G551D | AAATAATGGAGATGC | ACACCAAGATACGGG | AAATAATGGAGATGC |
| | AATGTTCAA (SEQ ID | CACAGAT (SEQ ID NO: 46) | AATGTTCAA (SEQ ID |
| | NO: 45) | | NO: 47) |
| HBB | CCAACTCCTAAGCCAG | AGTCAGTGCCTATCAG | CCAACTCCTAAGCCAG |
| | TGCCAGAAGAG (SEQ | AAACCCAAGAG (SEQ | TGCCAGAAGAG (SEQ |
| | ID NO: 48) | ID NO: 49) | ID NO: 50) |
| CCR5 | GCACAGGGTGGAACA | CACCACCCCAAAGGT | GCACAGGGTGGAACA |
| | AGATGG (SEQ ID NO: 51) | GACCGT (SEQ ID NO: 52) | AGATGG (SEQ ID NO: 53) |
| TREX1 | ATCACAGGTCTGAGCA | TGACAGCAGATGGTCT | ATCACAGGTCTGAGCA |
| | CAGC (SEQ ID NO: 54) | TGGC (SEQ ID NO: 55) | CAGC (SEQ ID NO: 56) |

PCR was performed using Q5 High Fidelity 2× Master Mix (New England Biolabs, M0492) with the following temperature and times.

| | Temp/minutes | | | | | | |
|---|---|---|---|---|---|---|---|
| Gene | Temp/minutes | | | | | | |
| Site | 1 | 2 Denature | 3 Anneal | 4 Extend | 5 | 6 | 7 |
| F508del | 98° C./3:00 | 98° C./0:30 | 58° C./0:20 | 72° C./0:35 | Repeat | 72° C./5:00 | 12° C./∞ |
| | | | | | from step | | |
| | | | | | 2 35X | | |
| G551D | 98° C./3:00 | 98° C./0:30 | 62° C./0:20 | 72° C./0:20 | Repeat | 72° C./5:00 | 12° C./∞ |
| | | | | | from step | | |
| | | | | | 2 35X | | |
| HBB | 95° C./3:00 | 95° C./0:30 | 62° C./0:20 | 72° C./0:15 | Repeat | 72° C./5:00 | 12° C./∞ |
| | | | | | from step | | |
| | | | | | 2 34X | | |
| CCR5 | 98° C./3:00 | 95° C./0:30 | 61° C./0:20 | 72° C./0:20 | Repeat | 72° C./5:00 | 12° C./∞ |
| | | | | | from step | | |
| | | | | | 2 34X | | |
| TREX1 | 95° C./3:00 | 95° C./0:20 | 67° C./0:20 | 72° C./0:30 | Repeat | 72° C./5:00 | 12° C./∞ |
| | | | | | from step | | |
| | | | | | 2 34X | | |

The PCR product was Sanger sequenced then Interference of CRISPR Edits (ICE) analysis was used to determine the percentage of corrected alleles (Synthego, ice.synthego.com, last accessed on Apr. 5, 2024).

Air-Liquid Interface. Gene corrected ABCs were plated on 6.5 mm Transwell plates with 0.4 μm pore polyester membrane insert (StemCell Technologies, 38024) at 40,000-50,000 cells per well. Pnuemacult Ex Plus and ROCK inhibitor (10 μM) was added to both the apical and basal side of insert and iMatrix was added to the apical insert to support expansion. Once cells became confluent, about 3-4 days, media was removed and ALI medium from the University of North Carolina (UNC media) was added to the basal side of the insert. UNC medium was replaced every other day for four weeks.

Immunostaining for epithelial cell characterization. CF-donor cells edited with esDNA and differentiated on ALI transwells for a duration of 4 weeks were stained for cytokeratin 5 (KRT5), acetylated tubulin (ACT), and mucin 5B (MUC5B) to identify basal, ciliated, and secretory cells, respectively. The cells were washed with F-12 (1×) Nutrient Mixture (Ham) (Gibco; 11765054) for 10 minutes on ice. The ALIs were fixed in a solution of 3% sucrose and 4% paraformaldehyde in PBS for a duration of 20 minutes on ice and then washed with PBS. Mouse anti-ACT (Sigma-Aldrich; T6793) was diluted 1:8000 and Rabbit anti-MUC5B (Sigma-Aldrich; HPA008246) was diluted 1:500 in staining buffer. 200 μL of the diluted primary antibodies were added to the ALI transwells and incubated overnight at 4° C. The primary antibodies were removed, ALI transwells were washed with PBS, and 200 μL of secondary antibody was added to the ALI transwells. PE Goat anti-mouse IgG (Biolegend; 405307) was diluted 1:200 and AF488 Donkey anti-rabbit (Life Technologies; A21206) was diluted 1:200 in staining buffer. The ALI transwells were incubated in secondary antibody for a duration of 1 hour. The secondary antibodies were removed, washed with PBS, and Recombinant Alexa Fluor 647 anti-Cytokeratin 5 antibody diluted 1:200 (Abcam, ab 193895) and DAPI (Sigma-Aldrich; D9542) diluted 1:1000 in staining buffer were added to the ALI transwells and incubated for 1 hour. The ALI transwells were washed with PBS, placed on slides using Fluoromount-G Mounting Medium (Invitrogen; 00-4958-02), and cover slipped.

Microscopy. Transwell slides were imaged on a motorized Eclipse Ti2-E inverted microscope (Nikon Instruments) with a SOLA LED engine (Lumencor) and an ORCA Fusion CMOS camera (Hamamatsu). Semrock filter sets were used for individual DAPI, GFP, TRITC, and Cy5 channels. Multichannel images were captured as Z-stacks using NIS-Elements AR software (Nikon Instruments, v. 5.30) with 40× Plan Apochromat Lambda objectives (Nikon Instruments) for a final resolution of 0.32 μm/pixel and 0.16 μm/pixel, respectively. The Z-stacks were compressed into 2D images using the Extended Depth of Focus (EDF) module in NIS-Elements.

Ussing Chamber Functional Assay. Four weeks after cells were plated in the transwell plates, ion channel function of CFTR was assessed with the Ussing chamber system (VCC MC6 from Physiologic Instruments Inc. San Diego, CA) with the U2500 Self-contained Ussing chambers (Warner Instruments, Hamden CT). To measure Cl⁻ secretion, the fully-differentiated epithelial sheets were placed with mucosal solution on apical side [120 mM NaGluconate, 25 mM NaHCO₃, 3.3 mM KH2PO4, 0.8 mM K2HPO4, 4 mM Ca(Gluconate)2, 1.2 mM Mg(Gluconat) 2, 10 mM mannitol] and serosal solution on basal side [120 mM NaCl, 25 mM NaHCO₃, 3.3 mM KH2PO4, 0.8 mM K2HPO4, 1.2 mM CaCl2, 1.2 mM MgCl2, 10 mM Glucose]. Following this, ion channel activators and inhibitors were added as follows: 10 μM amiloride apically to inhibit Epithelial Sodium Channel (ENaC), 10 μM forskolin bilaterally (Abcam #ab120058) to activate CFTR channels via activation of adenylyl cyclase concentrations, 10 μM CFTR potentiator drug VX-770 (SelleckChem, #S1144) apically, 10 μM CFTRinh-172 (Sigma Millipore, #C2992) apically to inhibit CFTR channel, and 100 μM uridine-5′-triphosphate trisodium (UTP) salt (Thermo Scientific Chemicals #AAJ63427MC) apically.

Staining and Fluorescence Activated Cell Sorting (FACS) Analysis. Cells were stained 3-4 days after editing with anti-Cytokeratin 5 antibody [EP1601Y] (Alexa Fluor® 647) (Abcam, ab193895), Rabbit IgG, monoclonal [EPR25A]-Isotype Control (Alexa Fluor® 647) (Abcam, ab 199093), Purified Mouse IgG2b k Isotype Ctrl (Biolegend, 401202), and purified anti-TP63 (W15093A) (Biolegend, 687202). Flow cytometry was done on the LSR/Fortessa cytometer and data was analyzed using FlowJo software (FlowJo, LLC).

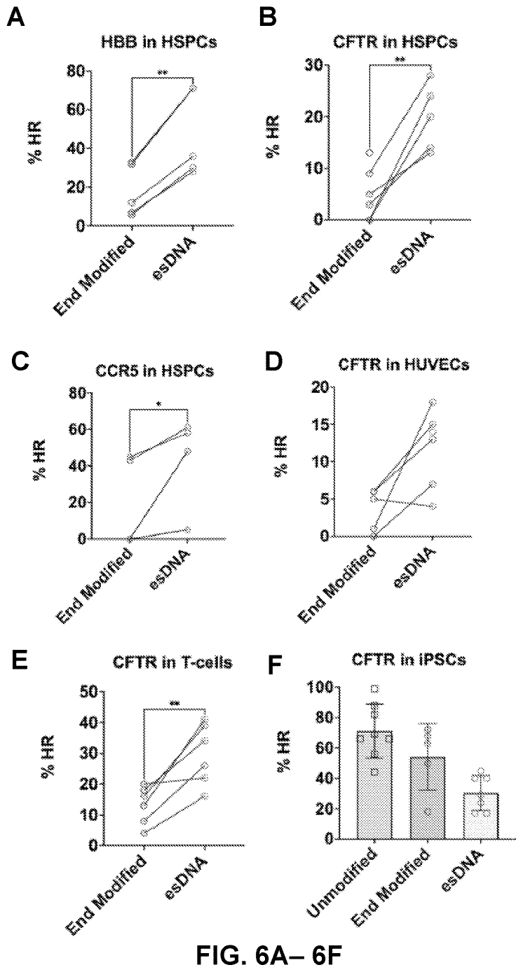

Statistical Analyses. For experiments comparing editing efficiencies using more than two types of ssDNA templates and/or AAV, significance was assessed using One Way Analysis of Variance (ANOVA) followed by multiple comparisons using Tukey's test. Paired T-test (two-tailed) was performed for results in FIGS. 6A-6E.

### Results

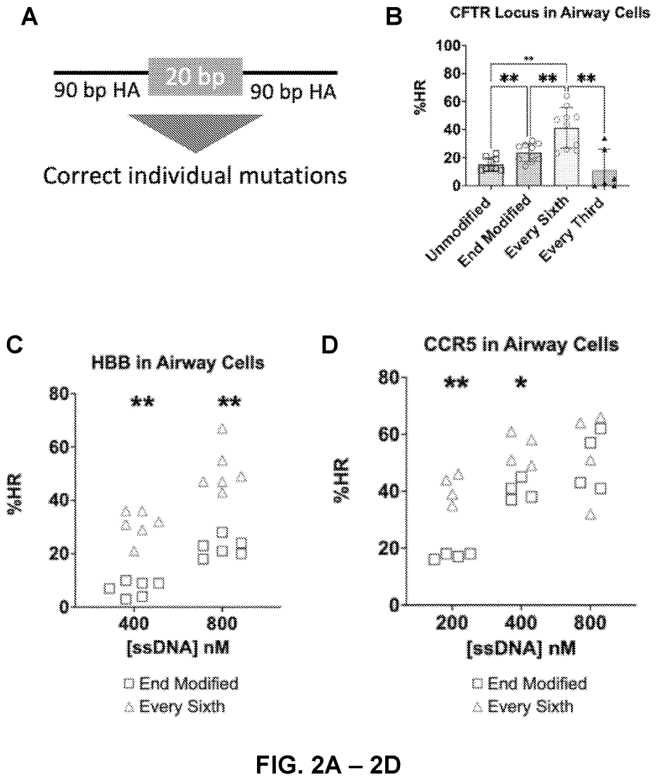

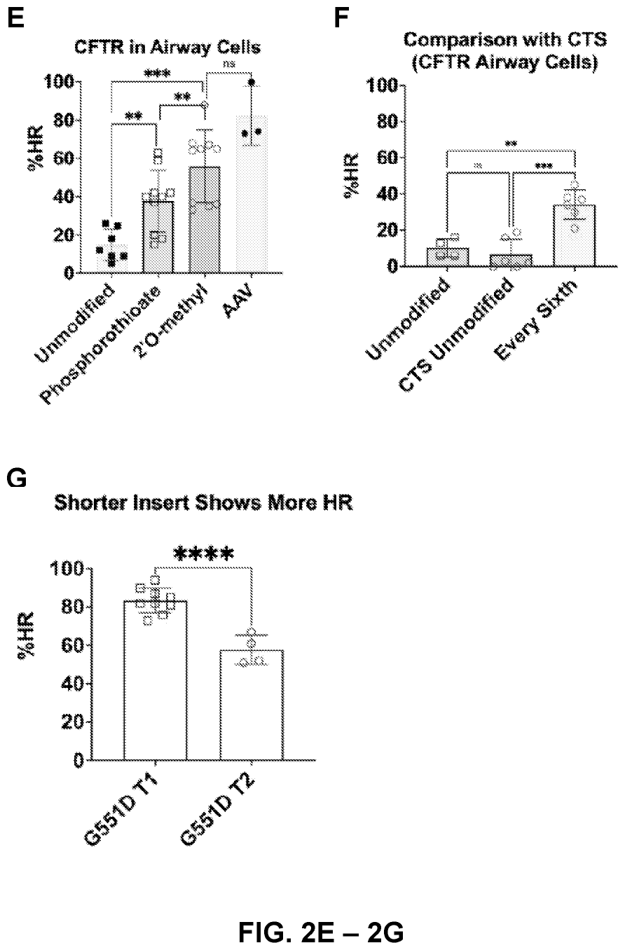

Primary airway basal stem cells obtained from explanted lungs or tissue discarded from endoscopic sinus surgeries were edited using ssDNA with different levels of chemical modifications. These HR templates were 200 bp long and introduced ~20 bp sequences into the target loci (FIG. 2A). These templates are ideal for correcting individual disease-causing mutations (e.g., sickle cell disease, cystic fibrosis, alpha1 anti-trypsin deficiency). It was observed that the ssDNA templates enable successful editing when combined with inhibition of DNA-PKc which is involved in NHEJ. Moreover, HR rates in the CFTR locus, CCR5 locus and hemoglobin (HBB) locus were all improved when editing was performed with ssDNA that was modified at every sixth base (FIGS. 2B-2C). Given these results, additional bases were modified in the ssDNA to improve stability further. However, modifying every third base resulted in worse HR in the CFTR locus (FIG. 2B).

It was also unexpectedly observed that ssDNAs that were modified at the ends (three nucleotides at the 3′ and 5′ ends) and every third base in the middle section (a 33.3% modification excluding the 3′ and 5′ ends of the template) showed worse gene editing efficiency than ssDNAs that were modified at the ends (three nucleotides at the 3′ and 5′ ends) and at every sixth base (16.7% modification excluding the 3′ and 5′ ends of the template). Without being bound by a particular theory, it is believed that even though extensive modification (e.g., modifying every third base, every second base or every base) of an ssDNA may prevent degradation of the ssDNA by exonucleases and endonucleases, such extensive modification of the template negatively effects homology directed repair efficiency.

Modifying ssDNA with 2′-O-methyl bases also improves editing in airway stem cells. Next, an alternate chemical modification (2′-O-methyl) was tested in the instant ssDNA templates. Modifying ssDNA in every sixth base pair with 2′O-methyl groups resulted in improved HR rates compared to unmodified ssDNA (FIG. 2E).

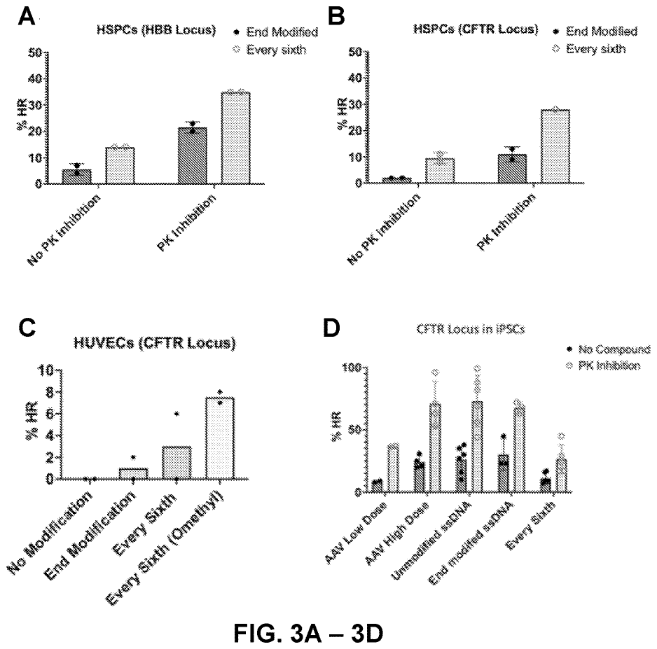

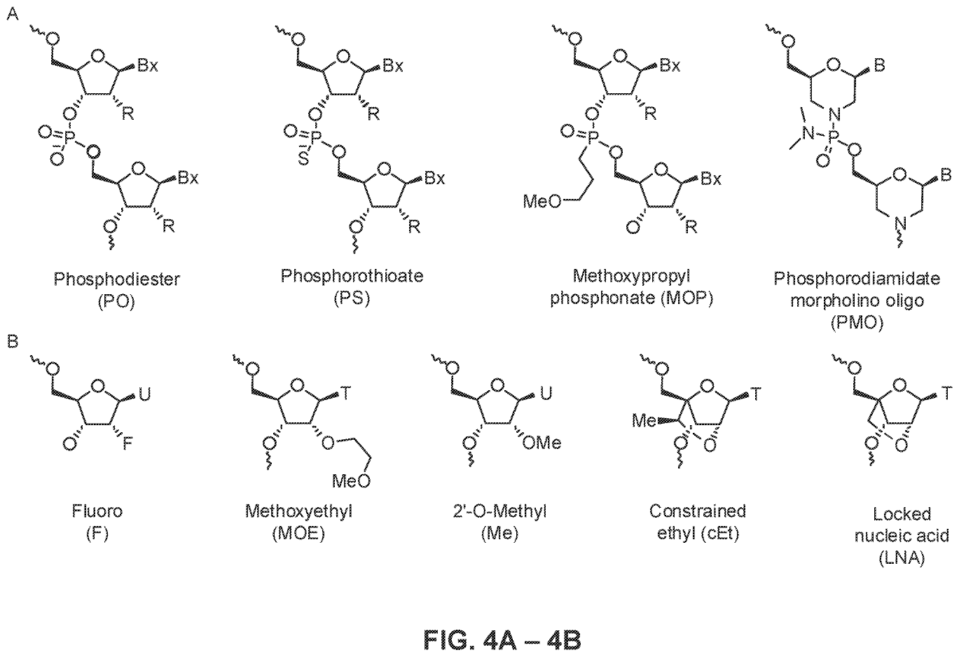

Chemically modified ssDNA improves editing in the HSPCs and HUVECs but not IPSCs. HSPCs from two different donors were edited in the HBB and CFTR loci, respectively. Single stranded DNA modified in every sixth base pair showed improved editing in both loci in cells from both donors (FIGS. 3A-3B). Human umbilical vein vascular endothelial cells (HUVEC) were edited in the CFTR locus and ssDNA with modifications in every sixth base pair showed improved editing compared to unmodified or end modified ssDNAs. The use of O-methyl modification in every sixth base pair further improved editing compared to the use of phosphorothioate modification (FIG. 3C). Lastly, three different iPSC lines were edited using ssDNA containing different levels of chemical modification. Unmodified ssDNA enabled highly efficient HR comparable to AAV based templates in induced pluripotent stem cells (iPSCs). HR rates were reduced when chemically modified ssDNA was used (FIG. 3D).

### Example 2: Testing the Impact of Chemically Modified ssDNA on Innate Immune Responses (Prophetic Example)

HSPCs edited with Cas9 and AAV lose their regenerative potential. This is believed to be mediated by the expression of DNA damage response proteins P53 and P21.

HSPCs edited using chemically modified ssDNA are expected to exhibit lower levels of P53 and P21 compared to HSPCs edited using AAVs. Levels of P21 mRNA are measured in airway cells, T-cells and HSPCs using quantitative PCR. Western blots and enzyme linked immunosorbent assays are used to measure the levels of p21 protein in edited airway cells, T-cells and HSPCs.

### Example 3: Extensive Chemical Modifications Improve Gene Insertion in Airway Cells

To test the hypothesis that extensive chemical modification of ssDNA may improve gene editing, ssDNA templates with phosphorothioate (PS) modifications in the last three bases (end-modified), at every sixth and every third base in addition to the three bases at the ends were used (FIGS. 1B-1C). Exon 11 was targeted in the CFTR locus and attempted to incorporate silent mutations in the region associated with the F508del mutation that is commonly seen in people with CF. Non-CF ABCs were edited with ssDNA templates that were either unmodified or chemically modified. Gene editing using ssDNA was inefficient (<10% alleles with HR) when ssDNA templates were used at a concentration of 800 nM in the absence of the DNA-PKcs inhibitor AZD-7648 and there was no improvement in gene insertion at higher concentrations of ssDNA. In the presence of AZD-7648, ssDNA templates with PS modification in every sixth base had higher HR (41±15%) than both unmodified (15±5%) and end-modified (24±6%) ssDNA templates (FIG. 2B). Optimal gene insertion was achieved using ssDNA templates modified at every sixth base at a concentration of 800 nM. Interestingly, ssDNA templates with PS modification in every third base showed a significant reduction in HR, suggesting an intermediary level of modifications is required to achieve optimal HR. The chemically modified ssDNA did not significantly reduce the proliferation of the edited cells when compared to unedited cells.

Cas9 Targeting Sequences (CTS) were recently reported to improve gene insertion using ssDNA by improving nuclear localization of the ssDNA. ssDNA templates were designed with CTS sequences targeting exon 11 of CFTR. Compared to unmodified ssDNA with CTS (7±9%), ssDNA with PS modifications in every sixth base showed significantly improved gene insertion (34±9%) (FIG. 2F). Adding CTS sequences to ssDNA with chemical modifications in every sixth base did not improve gene insertion further.

To test if these findings can be widely applied in other therapeutically relevant loci, Applicant tested the approach in the HBB and CCR5 loci using ssDNA concentrations ranging from 200-800 nM. Silent mutations were introduced in the region corresponding to the sickle cell mutation (E6V) in HBB and introduced a premature stop codon in the CCR5 locus. For HBB, ssDNA modified in every sixth base (esDNA) showed improved HR at all concentrations (FIG. 2C). For CCR5, esDNA improved HR at 200 and 400 nM but not 800 nM (FIG. 2D). Overall, esDNA showed significantly higher HR at all loci tested in ABCs with DNA-PKcs inhibition (FIGS. 2B, 2C, 2D, 2F). Gene insertion using esDNA templates is also superior to end-modified ssDNA when both DNA-PKcs and PolT are inhibited.

Applicant then investigated if improved editing can be obtained using other chemical modifications. esDNA with 2′O-methyl (OM) modifications was used on approximately every sixth base (FIG. 1C). Modifying thymine was avoided since the OM bases are RNA. OM modification in esDNA produced even higher HR (55±19%) than the esDNA with PS modification (38±16%). Gene insertion with OM modified esDNA was not significantly different from gene insertion using AAV templates (82±15%) (FIG. 2E). Both esDNA formulations had higher HR than unmodified ssDNA templates (15±8%).

To further understand if higher gene insertion levels can be achieved with shorter inserts, Applicant edited exon 12 in the CFTR locus associated with the variant G551D (another common CF causing mutation) with two different esDNA templates (FIG. 2G). Template 1 which replaces 12 bases next to the DSB site enabled gene insertion in 80-90% alleles (FIG. 2G). Conversely, template 2 which replaces a 34 bases segment yields ~60% edited alleles (FIG. 2G).

### Example 4: Gene Correction of the F508Del Mutation in CF ABCs Restores CFTR Function in Differentiated Airway Epithelial Cells

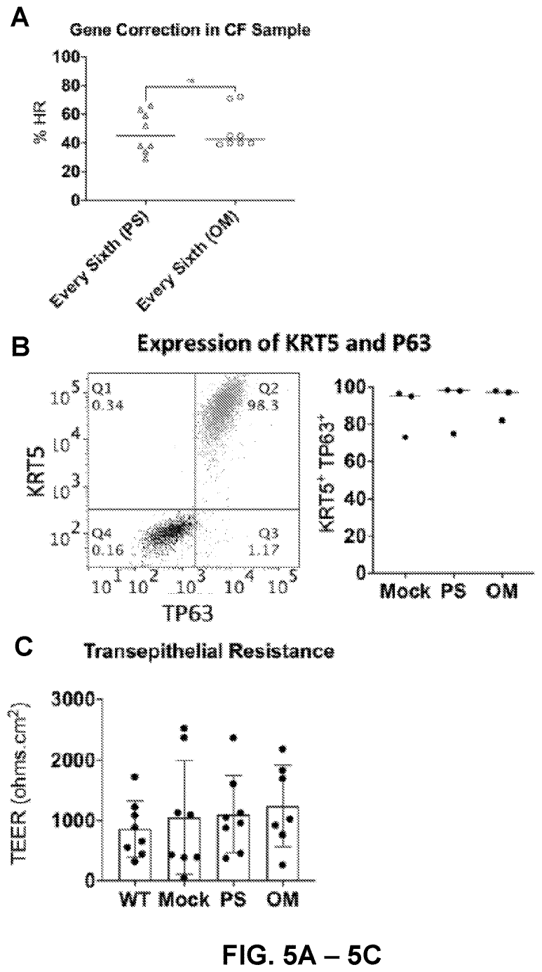

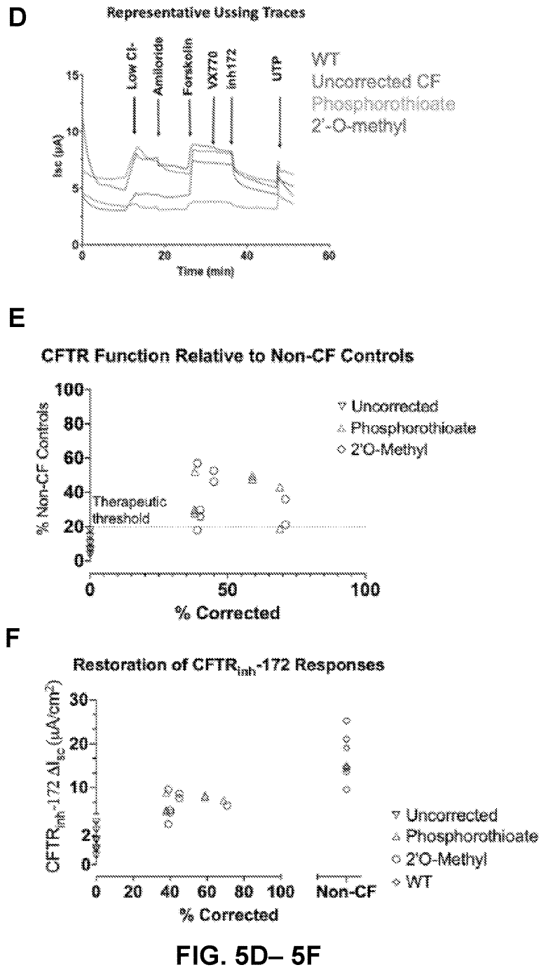

To test the ability to restore CFTR function, Applicant began by gene correcting ABCs homozygous for the F508del mutation. Patient-derived CF ABCs were efficiently corrected using esDNA with PS and OM modifications (FIG. 5A). In contrast to the non-CF ABCs, there was no significant difference between esDNA templates with PS or OM (47±14% vs 49±14%). Applicant further validated that esDNA did not result in the introduction of unintended missense mutations in the F508del locus using next-generation sequencing. Over 90% of alleles with HR (42% of total alleles) showed perfect insertion of the intended sequence. Editing did not induce a reduction in basal cell markers, cytokeratin 5 (KRT5) (Genecards ID: KRT5, HGNC: 6442 NCBI Gene: 3852 Ensembl: ENSG00000186081 OMIM®: 148040 UniProtKB/Swiss-Prot: P13647) and tumor protein P63 (P63) (Genecards ID: TP63, HGNC: 15979 NCBI Gene: 8626 Ensembl: ENSG00000073282 OMIM®: 603273 UniProtKB/Swiss-Prot: Q9H3D4) (FIG. 5B). On average, all conditions showed >85% cells that were positive for both KRT5 and P63 (KRT5 and P63 are markers for airway stem cells, e.g., distal airway stem cells).

To quantify the function of CFTR following gene correction, corrected CF, uncorrected CF, and non-CF samples, were grown at air liquid interface (ALI) to induce differentiation into polarized airway epithelial sheets and then currents were measured using Ussing chamber assay. Epithelial barrier integrity was evaluated by measuring the transepithelial resistance (TEER). Edited ABCs produced epithelial sheets with TEER comparable to controls thus indicating that the edited ABCs can form differentiated epithelial sheets (FIG. 5C). ALI cultures contained basal cells expressing KRT5, ciliated cells expressing acetylated tubulin (ACT) and secretory cells producing mucin 5B (MUC5B). The corrected CF samples showed restored CFTR function indicated by increased forskolin-stimulated currents and increased CFTRinh-172-sensitive short circuit current (FIG. 5D-5F). On average, uncorrected CF samples showed 1.6±0.9 μA/cm² change in short circuit currents in response to CFTRinh-172 compared to 16±6 μA/cm² for non-CF controls. CF samples corrected using PS and OM esDNA showed 6±2 μA/cm² change in short circuit currents in response to CFTRinh-172. This change in current was statistically significant when compared using one-way analysis of variance (ANOVA) followed by multiple comparisons test (p<0.05). When Applicant compared CFTR function in gene corrected cells to non-CF controls, epithelial sheets derived from ABCs edited using esDNA with PS and OM showed CFTR function above the therapeutic threshold (20%) for clinical benefit (37±12% for esDNA with PS and 36±15% for esDNA with OM relative to non-CF controls) (FIG. 5E).

### Example 5: Extensive Chemical Modification in ssDNA Templates Improves Gene Insertion in HSPCs, T-Cells, and HUVECs but not iPSCs

To further test if esDNA would be effective in improving gene insertion in other therapeutically relevant primary cells, Applicant tested them in HSPCs, HUVECs, T-cells, and iPSCs. In HSPCs, Applicant tested editing in the HBB, CFTR, and CCR5 loci and found that esDNA improved HR in all three loci. In HSPCs, esDNA (47±22%) improved HR over end-modified ssDNA (18±13%) in the HBB locus (FIG. 6A). Similar improvement in gene insertion was seen in CFTR (20±6% vs 5±5% for esDNA vs end-modified) and CCR5 (38±26% vs 15±23% for esDNA vs end-modified) loci in HSPCs (FIGS. 6B-6C). In HSPCs, gene insertion by esDNA templates was also more efficient compared to end-modified ssDNA in the absence of DNA-PKcs inhibition. esDNA were tested in HUVECs and T-cells by adding silent mutations in the CFTR locus. In HUVECs, the use of esDNA resulted in 12±5% alleles with HR compared to 4±3% alleles with HR obtained with end-modified ssDNA (FIG. 6D). In T-cells, esDNA templates resulted in 30±10% compared to 13±6% obtained with end-modified ssDNA (FIG. 6E).

Also, silent mutations were added in the CFTR locus in iPSCs using esDNA. Surprisingly, in iPSCs, unmodified ssDNA was more effective in enabling HR-mediated gene insertion over end-modified ssDNA (71±18% vs 54±22%). Conversely, esDNA showed reduced HR (30±12%) compared to all other conditions (FIG. 6F). Similar results were observed when iPSCs were edited in the absence of DNA-PKcs inhibition.

### Example 6: Knocking Out TREX1 Exonuclease Improves HR Using Unmodified ssDNA Templates

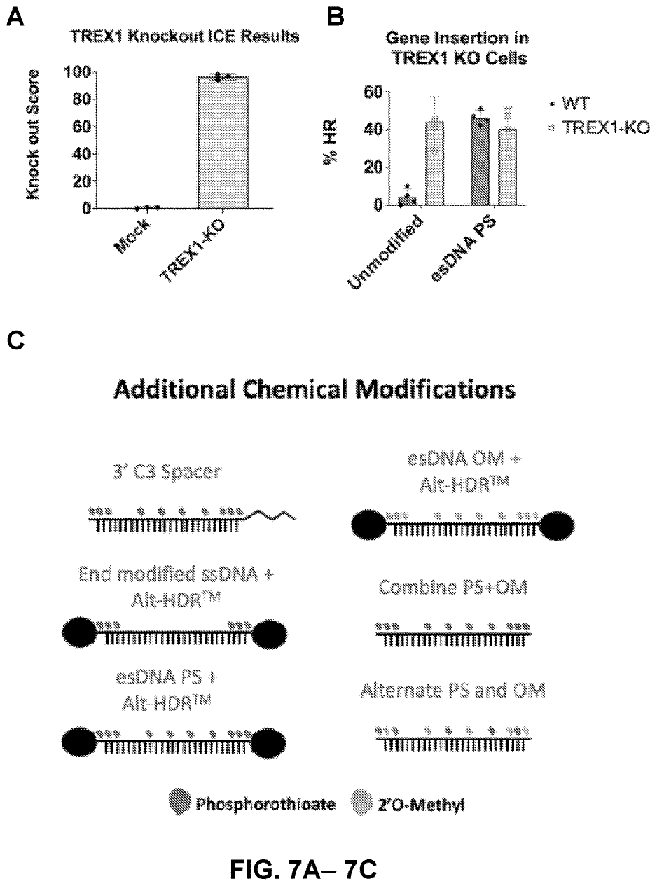

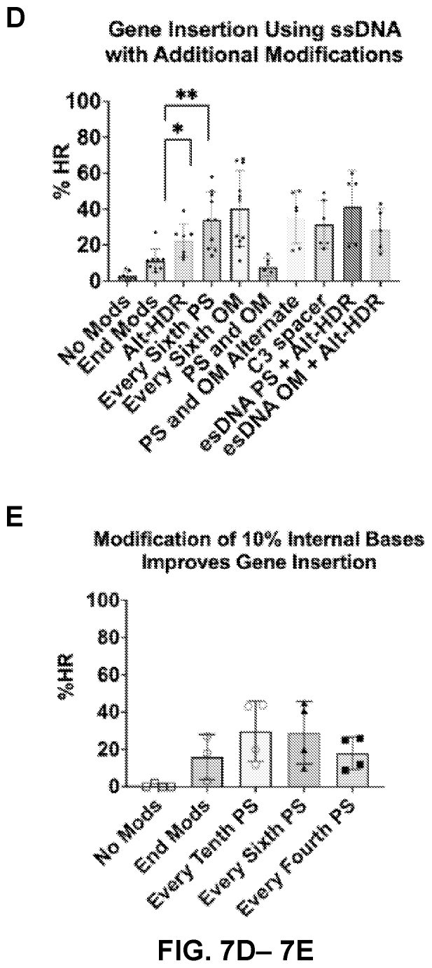

Next, the mechanism by which extensive chemical modifications protect ssDNA was investigated. To investigate the effect of TREX1 on ssDNA, ABCs were genome edited to knock out TREX1 (TREX1 KO) by using Cas9 to create insertions and deletions in the gene (FIG. 7A). HR using unmodified ssDNA was dramatically improved in TREX1 KO ABCs compared to wild type (WT) (4±4% for WT and 44±14% for TREX1-KO). However, HR was not improved in TREX1 KO ABCs when esDNA template was used (46±4% for WT and 41±11% for TREX1 KO) (FIG. 7B). HUVECs and T-cells with TREX1 KO also showed improved gene insertion using unmodified ssDNA. These studies suggest that extensive chemical modifications protect esDNA from TREX1.

Since modification of the 3′ end has been reported to protect ssDNA from TREX1, Applicant tested if protection of 3′ end by modifying the OH group in esDNA would improve gene insertion further (FIG. 7C). The addition of a 3′ C3 spacer to esDNA did not improve gene insertion (FIG. 7D). Applicant also investigated if proprietary end protecting modifications available from Integrated DNA Technologies (IDT) (Alt-HDR modifications) may enhance HR further. Alt-HDR modifications added to end-modified ssDNA improved gene insertion relative to end modification with PS alone (FIG. 7D). The use of esDNA still showed higher gene insertion relative to Alt-HDR modifications alone. However, there was no further improvement in gene insertion when Alt-HDR modifications were added to esDNA. Without being bound by theory, it is possible that increased protection of ssDNA by combining modifications may slow degradation by ssDNA further. Therefore, Applicant further investigated if using templates with combinations of PS and OM modifications improved gene insertion further (FIG. 7C). However, this was not the case (FIG. 7D). Since modification of every third base reduced gene insertion, Applicant wanted to assess if modifying fewer internal bases than every sixth would further improve gene insertion. Contrary to earlier predictions, modifying every tenth base in ssDNA resulted in 30±16% alleles with HR which was similar to esDNA (29±17%) (FIG. 7E). Modifying every fourth base reduced alleles with HR to 18±9%.

In order to further validate that the activity of TREX1 was specific to ssDNA, the impact of TREX1 on AAV-based HR templates targeting the CFTR locus was tested. These AAV templates have significantly longer homology arms (>800 bp) compared to the ssDNA and esDNA templates (~90 bp). AAV-based HR templates can be used to reliably edit the CFTR locus in ABCs, HSPCs, HUVECs and iPSCs. When using DNA-PKcs inhibition, Applicant obtained ~90% modified alleles in airway cells and 60-80% modification in HSPCs using AAV-based templates. ABCs with TREX1 KO did not show any change in gene insertion of the CFTR locus when edited using Cas9 and AAV-based templates in the presence of DNA-PKcs inhibition. Thus, the activity of TREX1 is specific to ssDNA.

### Example 7: Lipid Nanoparticle Delivery of Modified HR Templates

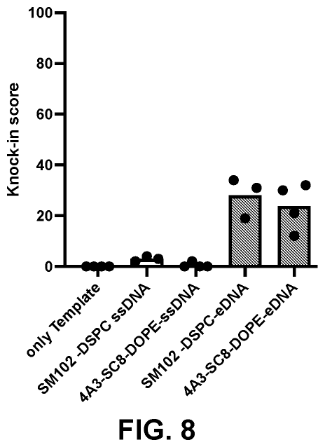

In subsequent experiments, Applicant attempted to test if eDNA templates show improved gene insertion when delivered along with Cas9 mRNA and sgRNA using lipid nanoparticles. Similar to prior Examples, exon 11 of CFTR which contains the F508del mutation was modified. eDNA templates were compared with unmodified ssDNA templates. The eDNA templates contained phosphorothioate modifications in every 6th base in addition to the three bases at the end. Cas9 mRNA, sgRNA and ssDNA (or eDNA) were packaged in lipid nanoparticle (LNPs) formulations previously published by others or from commercial kits (Cayman chemical, Cat: 35425). As shown in FIG. 8, gene insertion was superior in cells treated using LNPs carrying eDNA templates compared to unmodified ssDNA templates.

### Example 8

Previous studies investigating the use of ssDNA to perform HR-mediated gene insertion have explored the protection of the last 3 bases of the ssDNA templates but not internal bases. Although chemical modification of internal bases has been used to protect sgRNA and mRNA used in gene editing, the approach may not have been an obvious choice for HR templates due to differences in the biological processes involved. In the case of sgRNA, the modified bases do not interact with host proteins. Thus, modification of sgRNA has a lower chance of compromising nuclease activity as long as the modifications do not hinder base pairing or interactions with the nuclease. In the case of mRNA, many of the most successful modifications are already present in nature and thus compatible with the natural translational apparatus in cells. By way of contrast, chemical modifications in HR templates must be compatible with a variety of proteins involved in the HR pathway. The present results show, for the first time, that the HR machinery can indeed process chemically modified bases. The present results also show that this compatibility with the HR machinery can be lost if too many bases are modified.

Surprisingly, the modified bases do not appear to compromise the fidelity of HR. The next generation sequencing results show that the chemical modifications are processed similarly to normal bases by the HR machinery since significant unintended missense mutations were not present in the edited cells. Furthermore, CF airway cells corrected using esDNA templates show restored CFTR function (FIGS. 5D-5F).

AAV-based templates, which have significantly longer homology arms (>800 bp), can consistently achieve HR in up to 90% of alleles in the presence of DNA-PKcs inhibition (FIG. 2G). The present results show that the use of the right modification (FIG. 2G) and design allow the use of eDNA to modify a similar proportion of alleles as AAV templates (80-90% of alleles). It has previously been shown that gene insertion in the CFTR locus in airway cells can be increased by ~2-fold by increasing homology arm length to >150 bp. However, long chemically modified ssDNA templates are not available commercially and are challenging to produce.

The use of eDNA templates to achieve consistent gene insertion is improved by DNA-PKcs inhibitors that improve HR by blocking NHEJ. Applicant observed efficient gene insertion in ABCs using ssDNA templates in the presence of the DNA-PKcs inhibitor, AZD 7648. Applicant observed measurable HR in iPSCs and HSPCs without DNA-PKcs inhibition and the use of esDNA still improved gene insertion relative to ssDNA in HSPCs. Thus, although extensive chemical modification of ssDNA improves gene editing in multiple cell types, DNA-PKcs inhibition helps improve gene insertion using ssDNA or esDNA. It is possible that the differences in the efficiency of gene insertion correlate with the degradation of ssDNA in these cells because AAV-based templates enable similar gene modification in all the cell types tested.

### CONCLUSIONS

The modification of internal bases has been shown to improve sgRNA and mRNA activity. A similar approach has not been reported for improving HR using ssDNA templates. It was unclear if the strategy would improve gene insertion because the internal base modifications that inhibit nuclease degradation may also prevent the templates from being recognized by the proteins mediating HR. Furthermore, the approach could be rendered non-viable if the modified bases reduce the fidelity of the HR process and thus introduce missense mutations.

The present disclosure shows, for the first time, that modified internal bases (modified between every 4-10 bases) improve the protection of HR templates (e.g., an ssDNA or an RNA HR template) from nuclease-mediated degradation without effecting HR efficiency.

The present disclosure further demonstrates in three primary cell types that knocking out TREX1 can improve gene insertion using unmodified ssDNA templates. Moreover, since knocking out TREX1 did not improve gene insertion using AAV, the present results further show that the role of TREX1 in genome editing is specific to ssDNA. The present data shows that modifying internal bases along with the ends somehow further improves gene insertion. Notably, the present study also shows that the knockout of TREX1 can protect the ssDNA as efficiently as chemical modifications.

The esDNA templates described here can be extremely useful in characterizing the biological impact of small INDELS and missense mutations. Previous studies have attempted to model the biology of CFTR variants by introducing them into cell lines in vitro. Such models are valuable since they are isogenic and account for the impact of modifier genes that results in widely varying disease phenotypes. Similarly, Cas9 and ssDNA templates have been used to model the role of mutations in TP53 in myeloid malignancies in cell lines such as K562. Attempts have also been made to model oncogenic mutations in other genes in HSPCs and epithelial stem cells directly. The use of eDNA templates with DNA-PKcs inhibition may thus enable efficient generations of primary cells with such variants and aid such studies significantly.

Apart from modeling the impact of mutations, cell therapies have been proposed to treat genetic disorders caused by missense mutations and small INDELs such as sickle cell disease and CF. The ~40% allelic correction of the F508del mutation reported here enables the partial restoration of CFTR function (~35% of non-CF) in airway cells obtained from people with CF. This is therapeutically relevant because restoration of ~20% CFTR function relative to non-CF controls is thought to be sufficient to provide therapeutic benefit. Similarly, correction of 20-60% alleles in HSPCs from donors with sickle cell disease has been shown to restore mature hemoglobin production and prevent sickling. One advantage of short eDNA templates is that they can be readily produced in a few days as opposed to AAV template construction and packaging, which can take a few weeks to months. In addition, they may not elicit the innate immune responses which have been reported to compromise the regenerative potential of HSPCs edited using Cas9 and AAV.

Apart from biological reasons, non-viral templates that can be mass produced readily are also of interest for improving access to genetic therapies globally. Personalized ex-vivo genetic therapies require complex manufacturing of cells and AAV. In addition, they also require complex medical procedures that are not feasible in low-resource environments. In vivo gene editing using off-the-shelf reagents may enable widespread gene correction to treat genetic diseases globally. eDNA packaged in LNP may be used for correcting pathogenic mutations using in vivo gene editing. Non-viral in vivo editing approaches are less likely to be limited by immune responses which limit the efficacy of viral vectors such as AAV. LNPs targeting organs such as muscles which were traditionally only targetable using AAV have also been recently reported. Thus, the findings presented here can have broad therapeutic implications.

Other non-viral templates have been reported for gene insertion including ssDNA with CTS and double-stranded DNA with interstrand cross-linking. Applicant did not observe improved gene insertion using CTS in airway cells. It is possible that the observation may be due to the cell type or locus targeted. Compared to the CTS approach, eDNA does not require the design of special sequences and can thus be applied readily without additional effort or room for errors. Interstrand crosslinked dsDNA has not been reported for use in stem cells such as HSPCs and may be limited by innate immune responses that have been previously shown to limit gene insertion using dsDNA templates in primary cells. The results presented herein represent a further improvement on the efficiency of ssDNA templates using internal chemical modifications.

The present study shows that the homologous recombination machinery in primary cells can tolerate modified bases in the template. The results show that chemical modification of internal bases in ssDNA templates at every tenth to every sixth base significantly improves gene insertion using CRISPR-Cas9 in multiple clinically relevant human donor-derived primary cell types (HSPCs, T-cells, ABCs and HUVECs). Upon optimization, gene insertion can be achieved in 80-90% of alleles which is comparable to AAV mediated gene insertion. In addition, the present study shows that the chemical modifications work by protecting the ssDNA from degradation by TREX1. Overall, the present disclosure presents a broadly applicable method to improve the insertion of short sequences in primary cells using CRISPR-Cas9 and chemically modified ssDNA templates. These tools can enable improved production of cell therapies and enable studies characterizing the biological impact of different genetic variants in primary cells.

List of Sequences

SEQ ID NO: 1, protein, S. pyogenes Cas 9
MDKKYSIGLDIGTNSVGWAVITDDYKVPSKKFKVLGNTDRHSIKKNLIGALLFDSGE
TAEATRLKRTARRRYTRRKNRICYLQEIFSNEMAKVDDSFFHRLEESFLVEEDKKHE
RHPIFGNIVDEVAYHEKYPTIYHLRKKLVDSTDKADLRLIYLALAHMIKFRGHFLIEG
DLNPDNSDVDKLFIQLVQTYNQLFEENPINASGVDAKAILSARLSKSRRLENLIAQLP
GEKKNGLFGNLIALSLGLTPNFKSNFDLAEDAKLQLSKDTYDDDLDNLLAQIGDQYA
DLFLAAKNLSDAILLSDILRVNTEITKAPLSASMIKRYDEHHQDLTLLKALVRQQLPE
KYKEIFFDQSKNGYAGYIDGGASQEEFYKFIKPILEKMDGTEELLVKLNREDLLRKQ
RTFDNGSIPHQIHLGELHAILRRQEDFYPFLKDNREKIEKILTFRIPYYVGPLARGNSRF
AWMTRKSEETITPWNFEEVVDKGASAQSFIERMTNFDKNLPNEKVLPKHSLLYEYFT
VYNELTKVKYVTEGMRKPAFLSGEQKKAIVDLLFKTNRKVTVKQLKEDYFKKIECF
DSVEISGVEDRFNASLGTYHDLLKIIKDKDFLDNEENEDILEDIVLTLTLFEDREMIEE
RLKTYAHLFDDKVMKQLKRRRYTGWGRLSRKLINGIRDKQSGKTILDFLKSDGFAN
RNFMQLIHDDSLTFKEDIQKAQVSGQGDSLHEHIANLAGSPAIKKGILQTVKVVDEL
VKVMGRHKPENIVIEMARENQTTQKGQKNSRERMKRIEEGIKELGSQILKEHPVENT
QLQNEKLYLYYLQNGRDMYVDQELDINRLSDYDVDHIVPQSFLKDDSIDNKVLTRS
DKNRGKSDNVPSEEVVKKMKNYWRQLLNAKLITQRKFDNLTKAERGGLSELDKAG
FIKRQLVETRQITKHVAQILDSRMNTKYDENDKLIREVKVITLKSKLVSDFRKDFQFY
KVREINNYHHAHDAYLNAVVGTALIKKYPKLESEFVYGDYKVYDVRKMIAKSEQEI
GKATAKYFFYSNIMNFFKTEITLANGEIRKRPLIETNGETGEIVWDKGRDFATVRKVL
SMPQVNIVKKTEVQTGGFSKESILPKRNSDKLIARKKDWDPKKYGGFDSPTVAYSVL
VVAKVEKGKSKKLKSVKELLGITIMERSSFEKNPIDFLEAKGYKEVKKDLIIKLPKYS
LFELENGRKRMLASAGELQKGNELALPSKYVNFLYLASHYEKLKGSPEDNEQKQLF
VEQHKHYLDEIIEQISEFSKRVILADANLDKVLSAYNKHRDKPIREQAENIIHLFTLTN
LGAPAAFKYFDTTIDRKRYTSTKEVLDATLIHQSITGLYETRIDLSQLGGD
SEQ ID NO: 2, Protein, Artificial Sequence, NLS
PKKKRKV
SEQ ID NO: 3, Protein, Artificial Sequence, NLS
KRPAATKKAGQAKKKK
SEQ ID NO: 4, Protein, Artificial Sequence, NLS
PAAKRVKLD
SEQ ID NO: 5, Protein, Artificial Sequence, NLS
RQRRNELKRSP
SEQ ID NO: 6, Protein, Artificial Sequence, NLS
NQSSNFGPMKGGNFGGRSSGPYGGGGQYFAKPRNQGGY
SEQ ID NO: 7, Protein, Artificial Sequence, NLS
RMRIZFKNKGKDTAELRRRRVEVSVELRKAKKDEQILKRRNV
SEQ ID NO: 8, Protein, Artificial Sequence, NLS
VSRKRPRP
SEQ ID NO: 9, Protein, Artificial Sequence, NLS
PPKKARED
SEQ ID NO: 10, Protein, Artificial Sequence, NLS
POPKKKPL
SEQ ID NO: 11, Protein, Artificial Sequence, NLS
SALIKKKKKMAP
SEQ ID NO: 12, Protein, Artificial Sequence, NLS
DRLRR
SEQ ID NO: 13, Protein, Artificial Sequence, NLS
PKQKKRK
SEQ ID NO: 14, Protein, Artificial Sequence, NLS
RKLKKKIKKL
SEQ ID NO: 15, Protein, Artificial Sequence, NLS
REKKKFLKRR
SEQ ID NO: 16, Protein, Artificial Sequence, NLS
KRKGDEVDGVDEVAKKKSKK
SEQ ID NO: 17, Protein, Artificial Sequence, NLS
RKCLQAGMNLEARKTKK
SEQ ID NO: 18, DNA, Artificial Sequence
NNNNNNNNNNNNXGG
(N is A, G, T, or C; and X can be A, G, T, or C)
SEQ ID NO: 19, DNA, Artificial Sequence
NNNNNNNNNNNXGG
(N is A, G, T, or C; and X can be A, G, T, or C)
SEQ ID NO: 20, DNA, Artificial Sequence
NNNNNNNNNNNNXXAGAAW
(N is A, G, T, or C; X can be A, G, T, or C; and W is A or T)
SEQ ID NO: 21, DNA, Artificial Sequence
NNNNNNNNNNNXXAGAAW
(N is A, G, T, or C; X can be A, G, T, or C; and W is A or T)
SEQ ID NO: 22, DNA, Artificial Sequence
NNNNNNNNNNNNXGGXG
(N is A, G, T, or C; and X can be A, G, T, or C)
SEQ ID NO: 23, DNA, Artificial Sequence
NNNNNNNNNNNXGGXG
(N is A, G, T, or C; and X can be A, G, T, or C)
SEQ ID NO: 24, DNA, Artificial Sequence
NNNNNNNNNNNNNNNNNNNNgtttttgtactctcaagatttaGAAAtaaatcttgcagaagctacaaagataag
gcttcatgccgaaatcaacaccctgtcattttatggcagggtgttttcgttatttaaTTTTTT
SEQ ID NO: 25, DNA, Artificial Sequence
NNNNNNNNNNNNNNNNNNNNgtttttgtactctcaGAAAtgcagaagctacaaagataaggcttcatgccgaa
atcaacaccctgtcattttatggcagggtgttttcgttatttaaTTTTTT
SEQ ID NO: 26, DNA, Artificial Sequence
NNNNNNNNNNNNNNNNNNNNgtttttgtactctcaGAAAtgcagaagctacaaagataaggcttcatgccgaa
atcaacaccctgtcattttatggcagggtgtTTTTTT
SEQ ID NO: 27, DNA, Artificial Sequence
NNNNNNNNNNNNNNNNNNNNgttttagagctaGAAAtagcaagttaaaataaggctagtccgttatcaacttga
aaaagtggcaccgagtcggtgcTTTTTT
SEQ ID NO: 28, DNA, Artificial Sequence
NNNNNNNNNNNNNNNNNNNNgttttagagctaGAAATAGcaagttaaaataaggctagtccgttatcaactt
gaaaaagtgTTTTTTT
SEQ ID NO: 29, DNA, Artificial Sequence
NNNNNNNNNNNNNNNNNNNNgttttagagctagAAATAGcaagttaaaataaggctagtccgttatcaTT
TTTTTT
SEQ ID NO: 30, DNA, Artificial Sequence, FokI
gtcgcgggat cccaactagt caaaagtgaa ctggaggaga agaaatctga acttcgtcataaattgaaat atgtgcctca
tgaatatatt gaattaattg aaattgccag aaatcccactcaggatagaa ttcttgaaat gaaggtaatg gaatttttta
tgaaagttta tggatatagaggtgagcatt tgggtggatc aaggaaaccg gacggagcaa tttatactgt cggatctcct
attgattacg gtgtgatcgt ggatactaaa gcttatagcg gaggttataa tctgccaattggccaagcag atgaaatgga
gcgatatgtc gaagaaaatc aaacacgaga caaacatctgaaccctaatg aatggtggaa agtctatcca tottctgtaa
cggaatttaa gtttttatttgtgagtggtc actttaaagg aaactacaaa gctcagctta cacgattaaa
tcatatcactaattgtaatg gagctgttct tagtgtagaa gagcttttaa ttggtggaga aatgattaaa gccggcacat
taaccttaga ggaagtcaga cggaaattta ataacggcga gataaacttt taagggccct tcgaaggtaa gcctatccct
aaccctctcc tcggtctcga ttctacgcgt
accggtcatc at
SEQ ID NO: 31, DNA, Artificial Sequence, FokI
gtcgcgggat cccaactagt caaaagtgaa ctggaggaga agaaatctga acttcgtcataaattgaaat atgtgcctca
tgaatatatt gaattaattg aaattgccag aaatcccact caggatagaa ttcttgaaat gaaggtaatg gaatttttta
tgaaagttta tggatataga ggtgagcatt tgggtggatc aaggaaaccg gacggagcaa tttatactgt cggatctcct
attgattacg gtgtgatcgt ggatactaaa gcttatagcg gaggttataa tctgccaatt ggccaagcag atgaaatgca
acgatatgtc aaggaaaatc aaacacgaaa caaacatatc aaccctaatg aatggtggaa agtctatcca tcttctgtaa
cggaatttaa gtttttattt gtgagtggtc actttaaagg aaactacaaa gctcagctta cacgattaaa toggaagact
aattgtaatg gagctgttct tagtgtagaa gagcttttaa ttggtggaga aatgattaaa gccggcacat taaccttaga
ggaagtcaga cggaaattta ataacggcga gataaacttttaagggccct tcgaaggtaa gcctatccct aaccctctcc
tcggtctcga ttctacgcgt accggtcatc at
SEQ ID NO: 32, Protein, Artificial Sequence FokI
VAGSQLVKSELEEKKSELRHKLKYVPHEYIELIEIARNPTQDRILEMETKVMETEFFM
ETKVYGYRGEHLGGSRKPDGAIYTVGSPIDYGVIVDTKAYSGGYNLPIGQADEMETE
RYVEENQTRDKHLNPNEWWKVYPSSVTEFKFLFVSGHFKGNYKAQLTRLNHITNCN
GAVLSVEELLIGGEMETIKAGTLTLEEVRRKFNNGEINFLGPFEGKPIPNPLLGLDSTR
TGHH
SEQ ID NO: 33, Protein, Artificial Sequence FokI
VAGSQLVKSELEEKKSELRHKLKYVPHEYIELIEIARNPTQDRILEMETKVMETEFFMET
KVYGYRGEHLGGSRKPDGAIYTVGSPIDYGVIVDTKAYSGGYNLPIGQADEMETQRYVKE
NQTRNKHINPNEWWKVYPSSVTEFKFLFVSGHFKGNYKAQLTRLNRKTNCNGAVLSVEEL
LIGGEMETIKAGTLTLEEVRRKFNNGEINFLGPFEGKPIPNPLLGLDSTRTGHH
SEQ ID NO: 34, DNA, Artificial Sequence,
CCTGGATTATGCCTGGCACCATTAAAGAAAATATCATCTTcGGcGTgTCtTAcGAcG
AgTAcAGATACAGAAGCGTCATCAAAGCATGCCAACTAGAAGAGG
SEQ ID NO: 35, DNA, Artificial Sequence,
ATGCTATCTATATTCATCATAGGCCTGGATTATGCCTGGCACCATTAAAGAAAAT
ATCATCTTcGGcGTgTCtTAcGAcGAgTAcAGATACAGAAGCGTCATCAAAGCATGC
CAACTAGAAGAGGCCTATGATGAATATAGATAGCAT
SEQ ID NO: 36, DNA, Artificial Sequence,
ATGCTATCTATATTCATCATAGGCCTCTTCTAG
SEQ ID NO: 37, DNA, Artificial Sequence,
ATAATCCAGGCCTATGATGAATATAGATAGCAT
SEQ ID NO: 38, DNA, Artificial Sequence,
ATGGGAGAACTGGAGCCTTCAGAGGGTAAAATTAAGCACAGTGGAAGAATTTCA
TTCTGTTCTCAGTTTTCCTGGATTATGCCTGGCACCATTAAAGAAAATATCATCTT
cGGcGTgTCtTAcGAcGAgTAcAGATACAGAAGCGTCATCAAAGCATGCCAACTAGA
AGAGGTAAGAAACTATGTGAAAACTTTTTGATT
SEQ ID NO: 39, DNA, Artificial Sequence,
A*T*G*GGAGAACTGGAGCCTTCAGAGGGTAAAATTAAGCACAGTGGAAGAATTT
CATTCTGTTCTCAGTTTTCCTGGATTATGCCTGGCACCATTAAAGAAAATATCATC
TTcGGcGTgTCtTAcGAcGAgTAcAGATACAGAAGCGTCATCAAAGCATGCCAACTA
GAAGAGGTAAGAAACTATGTGAAAACTTTTTG* A*T*T
(* indicates that the preceding nucleotide is PS modified)
SEQ ID NO: 40, DNA, Artificial Sequence,
A*T*G*GGAGAA*CTGGAG*CCTTCA*GAGGGT*AAAATT*AAGCAC*AGTGGA*A
GAATT*TCATTC*TGTTCT*CAGTTT*TCCTGG*ATTATG*CCTGGC*ACCATT*AAA
GAA*AATATC*ATCTTc*GGcGTg*TCtTAc*GAcGAg*TAcAGA*TACAGA*AGCGTC*
ATCAAA*GCATGC*CAACTA*GAAGAG*GTAAGA*AACTAT*GTGAAA*ACTTTT*
TG*A*T*T
(* indicates that the preceding nucleotide is PS modified)
SEQ ID NO: 41, DNA, Artificial Sequence,
A*T*G*GGA*GAA*CTG*GAG*CCT*TCA*GAG*GGT*AAA*ATT*AAG*CAC*AGT*
GGA*AGA*ATT*TCA*TTC*TGT*TCT*CAG*TTT*TCC*TGG*ATT*ATG*CCT*GGC*
ACC*ATT*AAA*GAA*AAT*ATC*ATC*TTc*GGc*GTg*TCt*TAc*GAc*GAg*TAc*A
GA*TAC*AGA*AGC*GTC*ATC*AAA*GCA*TGC*CAA*CTA*GAA*GAG*GTA*AG
A*AAC*TAT*GTG*AAA*ACT*TTT*TG* A*T*T
(* indicates that the preceding nucleotide is PS modified)
SEQ ID NO: 42, DNA, Artificial Sequence
CCTTCTACTCAGTTTTAGTC
SEQ ID NO: 43, DNA, Artificial Sequence
TGGGTAGTGTGAAGGGTTCAT
SEQ ID NO: 44, DNA, Artificial Sequence
AGGCAAGTGAATCCTGAGCG
SEQ ID NO: 45, DNA, Artificial Sequence
AAATAATGGAGATGCAATGTTCAA
SEQ ID NO: 46, DNA, Artificial Sequence
ACACCAAGATACGGGCACAGAT
SEQ ID NO: 47, DNA, Artificial Sequence
AAATAATGGAGATGCAATGTTCAA
SEQ ID NO: 48, DNA, Artificial Sequence
CCAACTCCTAAGCCAGTGCCAGAAGAG
SEQ ID NO: 49, DNA, Artificial Sequence
AGTCAGTGCCTATCAGAAACCCAAGAG
SEQ ID NO: 50, DNA, Artificial Sequence
CCAACTCCTAAGCCAGTGCCAGAAGAG
SEQ ID NO: 51, DNA, Artificial Sequence
GCACAGGGTGGAACAAGATGG
SEQ ID NO: 52, DNA, Artificial Sequence
CACCACCCCAAAGGTGACCGT
SEQ ID NO: 53, DNA, Artificial Sequence
GCACAGGGTGGAACAAGATGG
SEQ ID NO: 54, DNA, Artificial Sequence
ATCACAGGTCTGAGCACAGC
SEQ ID NO: 55, DNA, Artificial Sequence
TGACAGCAGATGGTCTTGGC
SEQ ID NO: 56, DNA, Artificial Sequence
ATCACAGGTCTGAGCACAGC

### EQUIVALENTS

Unless otherwise defined, all technical and scientific terms used herein have the same meaning as commonly understood by one of ordinary skill in the art to which this disclosure belongs.

The disclosures illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the disclosure claimed.

Thus, it should be understood that although the present disclosure has been specifically disclosed by preferred embodiments and optional features, modification, improvement and variation of the disclosures embodied therein herein disclosed may be resorted to by those skilled in the art, and that such modifications, improvements and variations are considered to be within the scope of this disclosure. The materials, methods, and examples provided here are representative of preferred embodiments, are exemplary, and are not intended as limitations on the scope of the disclosure.

The disclosure has been described broadly and generically herein. Each of the narrower species and subgeneric groupings falling within the generic disclosure also form part of the disclosure. This includes the generic description of the disclosure with a proviso or negative limitation removing any subject matter from the genus, regardless of whether or not the excised material is specifically recited herein.

All publications, patent applications, patents, and other references mentioned herein are expressly incorporated by reference in their entirety, including all formulas and figures, to the same extent as if each were incorporated by reference individually. In case of conflict, the present specification, including definitions, will control.

Other embodiments are set forth within the following claims.

1. A gene-editing system comprising:
components of a gene editing system selected from a CRISPR-Cas system, a TALEN system and a Zinc Finger Nuclease (ZFN) system; and
a single stranded homology directed repair (HDR) template polynucleotide,
wherein the HDR repair template polynucleotide comprise a chemically modified nucleotide every 4, 5, 6, 7 or 8 nucleotides along at least 95% of the length of the HDR template polynucleotide.

2. The gene-editing system of claim 1, wherein the first three nucleotides at the 5′ end and the last three nucleotides at the 3′ end of the HDR repair template polynucleotide comprise a chemically modified nucleotide.

3. The gene-editing system of claim 1, wherein the single stranded HDR template comprises a transgene.

4. The gene-editing system of claim 1, wherein the chemically modified nucleotide comprises a phosphorothioate (PS) modification, a 2′-o-methyl modification, methoxypropyl phosphonate (MOP), a constrained ethyl (cEt) modification, a locked nucleic acid (LNA) modification, a 2′ fluoro modification, a 1-Boranotriphosphate modification, methoxypropyl phosphonate (MOP) modification, phosphorodiamidate morpholino oligomer (PMO) modification, or a combination thereof.

5. The gene-editing system of claim 1, wherein the single stranded HDR template is a single stranded DNA or RNA/DNA hybrid.

6. The gene-editing system of claim 1, wherein the gene-editing system comprises a CRISPR enzyme or a polynucleotide encoding a CRISPR enzyme, a guide RNA (gRNA) sequence that hybridizes to a target sequence, a tracr mate sequence, and a tracr sequence.

7. The gene-editing system of claim 1, wherein the gene-editing system comprises a nickase.

8. (canceled)

9. (canceled)

10. The gene-editing system of claim 1, wherein the gene-editing system comprises a TALEN system comprising, from the N-terminus to the C-terminus, a first spacer sequence, a TALE DNA binding domain, a second spacer sequence, and a FokI nuclease catalytic domain fused to the C-terminus, wherein the TALE DNA binding domain comprises TAL repeats, wherein the TALE DNA binding domain is specific for binding to target sequence.

11. The gene-editing system of claim 1, wherein the gene-editing system comprises a ZFN system comprising at least one zinc finger nuclease, the zinc finger nuclease comprising a zinc finger protein comprising 4, 5 or 6 fingers designated F1 to F4, F1 to F5 or F1 to F6, each finger comprising a recognition helix region that recognizes a target sequence.

12. A non-naturally occurring or engineered composition comprising:
(i) a polynucleotide sequence encoding a CRISPR enzyme,
(ii) a CRISPR-Cas system chimeric RNA (chiRNA) polynucleotide sequence comprising:
(a) a guide sequence capable of hybridizing to a target sequence,
(b) a tracr mate sequence, and
(c) a tracr sequence,
wherein (a), (b) and (c) are arranged in a 5′ to 3′ orientation,
wherein when transcribed, the tracr mate sequence hybridizes to the tracr sequence and the guide sequence directs sequence-specific binding of a CRISPR complex to the target sequence; and
(ii) a single stranded homology directed repair (HDR) template polynucleotide that comprises a chemically modified nucleotide every 4, 5, 6, 7 or 8 nucleotides along at least 95%, of the length of the HDR template polynucleotide.

13. (canceled)

14. (canceled)

15. (canceled)

16. The composition of claim 12, wherein the single stranded HDR template nucleic acid is a single stranded DNA or RNA/DNA hybrid.

17. (canceled)

18. (canceled)

19. The composition of claim 12, wherein the CRISPR enzyme is a nickase.

20. (canceled)

21. The composition of claim 19, further comprising:
(iv) a second CRISPR-Cas system chimeric RNA (chiRNA) polynucleotide sequence comprising:
(a) a second guide sequence capable of hybridizing to a second target sequence,
(b) a second tracr mate sequence, and
(c) a second tracr sequence,
wherein (a), (b) and (c) are arranged in a 5′ to 3′ orientation, and
wherein when transcribed, the second tracr mate sequence hybridizes to the second tracr sequence and the second guide sequence directs sequence-specific binding of a second CRISPR complex to the second target sequence.

22. (canceled)

23. The composition of claim 12, wherein the chemically modified nucleotide comprises a phosphorothioate (PS) modification, an o-methyl modification, methoxypropyl phosphonate (MOP), a constrained ethyl (cEt) modification, a locked nucleic acid (LNA) modification, a 2′ fluoro modification, a 1-Boranotriphosphate modification, methoxypropyl phosphonate (MOP) modification, phosphorodiamidate morpholino oligomer (PMO) modification, or a combination thereof.

24. A method for modifying an organism comprising delivering the gene-editing system of claim 1 to the organism.

25. A method for modifying the genome of a cell comprising delivering the gene-editing system of claim 1 to the cell.

26. (canceled)

27. A method for ex vivo cell therapy comprising administering a subject a cell prepared by the method of claim 25.

28. (canceled)

29. An isolated host cell comprising the gene-editing system of claim 1.

30. (canceled)

31. (canceled)

32. (canceled)

33. (canceled)

34. (canceled)

35. (canceled)

36. The gene editing system of claim 1, further comprising a pharmaceutically acceptable carrier.

37. The gene editing system of claim 1, further comprising instructions for use.
